(19) United States
(12) Patent Application Publication
(10) Pub. No.: US 2014/0315797 A1
(43) Pub. Date: Oct. 23, 2014

(54) Novel N-Terminally Modified Insulin Derivatives

(71) Applicants: Peter Madsen, Bagsvaerd (DK); Per Balschmidt, Hoersholm (DK); Svend Havelund, Bagsvaerd (DK); Thomas Hoeg-Jensen, Klampenborg (DK); Thomas Boerglum Kjeldsen, Virum (DK); Charlotte Harkjaer Fynbo, Herlev (DK)

(72) Inventors: Peter Madsen, Bagsvaerd (DK); Per Balschmidt, Hoersholm (DK); Svend Havelund, Bagsvaerd (DK); Thomas Hoeg-Jensen, Klampenborg (DK); Thomas Boerglum Kjeldsen, Virum (DK); Charlotte Harkjaer Fynbo, Herlev (DK)

(21) Appl. No.: 13/823,952

(22) PCT Filed: Oct. 14, 2011
(86) PCT No.: PCT/EP11/68019

§ 371 Date: Aug. 27, 2013

Related U.S. Application Data
(60) Provisional application No. 61/394,090, filed on Oct. 18, 2010.

(30) Foreign Application Priority Data
Oct. 15, 2010 (EP) 10187706.6

(51) Int. Cl. C07K 14/62

(52) U.S. Cl. CPC C07K 14/62
USPC 514/5.9; 530/303

(57) ABSTRACT

The invention is related to novel N-terminally modified insulin derivatives, pharmaceutical compositions comprising such and methods of making such.

### FIELD OF THE INVENTION

The present invention is related to novel N-terminally modified insulin derivatives and methods of making such.

### BACKGROUND OF THE INVENTION

Diabetes mellitus is a metabolic disorder in which the ability to utilize glucose is partly or completely lost. The disorder may e.g. be treated by administering insulin.

The oral route is by far the most widely used route for drug administration and is in general very well accepted by patients, especially for chronic therapies. Administration of insulin is however often limited to parenteral routes rather than the preferred oral administration due to several barriers such as enzymatic degradation in the gastrointestinal (GI) tract and intestinal mucosa, drug efflux pumps, insufficient and variable absorption from the intestinal mucosa, as well as first pass metabolism in the liver.

Some of the commercial available insulin formulations are characterized by a fast onset of action and other formulations have a relatively slow onset but show a more or less prolonged action. WO 08/034,881 describes protease stable insulin analogues and WO 2009/115469 relates to certain acylated insulin analogues wherein at least two hydrophobic amino acids have been substituted with hydrophilic amino acids. WO 2008/145721 is related to certain peptides which have been N-terminal modified to protect said peptides against degradation by aminopeptidases and dipeptidyl peptidases. WO 2010/033220 describes peptide conjugates coupled to polymers and optionally one or more moieties with up to ten carbon atoms.

Pharmaceutical compositions of therapeutic peptides are required to have a shelf life of several years in order to be suitable for common use. However, peptide compositions are inherently unstable due to sensitivity towards chemical and physical degradation. Chemical degradation involves change of covalent bonds, such as oxidation, hydrolysis, racemization or crosslinking. Physical degradation involves conformational changes relative to the native structure of the peptide, i.e. secondary and tertiary structure, such as aggregation, precipitation or adsorption to surfaces.

WO 08/145,728, WO 2010/060667 and WO 2011/086093 disclose examples of lipid pharmaceutical compositions for oral administration.

Pharmaceutical compositions often contain aldehyde and ketones in concentrations up to 200 ppm. Aldehyde and ketones may react with insulin and thus give rise to extensive chemical degradation of the insulin in the composition. As a result, the shelf life of the insulin composition may be below 3 months. Pharmaceutical drug development requires at least 2 years of shelf life.

It is known that aqueous pharmaceutical compositions can comprise compounds such as ethylenediamine for stability purposes. For example WO 2006/125763 describes aqueous pharmaceutical polypeptide compositions comprising ethylenediamine as a buffer.

However, a method remains to be found for stabilising insulin in pharmaceutical compositions, especially non-aqueous lipid compositions, without adding ethylene diamine or other stabilizing compounds to the composition.

### SUMMARY OF THE INVENTION

The invention is related to N-terminally modified insulin derivatives.

In an aspect of the invention, an N-terminally modified insulin is provided, wherein the insulin is an acylated, protease stabilised insulin and the N-terminal modification is with one or more N-terminal modification groups that are positively charged at physiological pH.

In an aspect of the invention, an N-terminally modified insulin is provided, wherein the insulin is an acylated insulin and the N-terminal modification is with one or more N-terminal modification groups that are neutral or negatively charged at physiological pH.

The invention also contemplates an oral pharmaceutical composition comprising one or more lipids and an N-terminally modified insulin.

Also methods of producing said N-terminally modified insulin derivatives are described.

### DESCRIPTION OF THE DRAWINGS

FIG. 1: Formation of impurities as measured by UPLC upon storage of the analogue of the prior art at different temperatures.

FIG. 2: Formation of HMWP (high molecular weight products) upon storage of the analogue of the prior art at different temperatures.

FIG. 3: Formation of impurities as measured by UPLC upon storage of the analogue of example 1 at different temperatures.

FIG. 4: Formation of HMWP (high molecular weight products) upon storage of the analogue of example 1 at different temperatures.

FIG. 5: Formation of impurities as measured by UPLC upon storage of the analogue of example 2 at different temperatures.

FIG. 6: Formation of HMWP (high molecular weight products) upon storage of the analogue of example 2 at different temperatures.

FIG. 7: Formation of impurities as measured by UPLC upon storage of the analogue of example 12 at different temperatures.

FIG. 8: Formation of HMWP (high molecular weight products) upon storage of the analogue of example 12 at different temperatures.

FIG. 9: Formation of impurities as measured by UPLC upon storage of the analogue of example 33 at different temperatures.

FIG. 10: Formation of HMWP (high molecular weight products) upon storage of the analogue of example 33 at different temperatures.

FIG. 11: Formation of impurities as measured by UPLC upon storage of the analogue of example 38 at different temperatures.

FIG. 12: Formation of HMWP (high molecular weight products) upon storage of the analogue of example 38 at different temperatures.

FIG. 13: Formation of impurities as measured by UPLC upon storage of the analogue of example 39 at different temperatures.

FIG. 14: Formation of HMWP (high molecular weight products) upon storage of the analogue of example 39 at different temperatures.

FIG. 15: Formation of impurities as measured by UPLC upon storage of the analogue of example 40 at different temperatures.

FIG. 16: Formation of HMWP (high molecular weight products) upon storage of the analogue of example 40 at different temperatures.

FIG. 17: Formation of impurities as measured by UPLC upon storage of the analogue of example 41 at different temperatures.

FIG. 18: Formation of HMWP (high molecular weight products) upon storage of the analogue of example 41 at different temperatures.

FIG. 19: Formation of impurities as measured by UPLC upon storage of the analogue of example 59 at different temperatures.

FIG. 20: Formation of HMWP (high molecular weight products) upon storage of the analogue of example 59 at different temperatures.

FIG. 21: Formation of impurities as measured by UPLC upon storage of the analogue of example 60 at different temperatures.

FIG. 22: Formation of HMWP (high molecular weight products) upon storage of the analogue of example 60 at different temperatures.

### DESCRIPTION OF THE INVENTION

The present invention is related to novel N-terminally modified insulins, also herein named N-terminally protected insulins, and methods of making such. The novel N-terminally modified insulins are particularly suitable for use in oral formulations. An aspect of the invention thus contemplates oral pharmaceutical compositions comprising N-terminally modified insulins.

It has surprisingly been found by the inventors that the insulins according to the invention are stable in pharmaceutical compositions comprising aldehydes and/or ketones, such as trace amounts thereof, while the biological and pharmacological properties of the insulins are retained when compared to parent insulins, i.e. the similar insulins without N-terminal modification.

In one aspect of the invention, N-terminally modified insulins according to the invention are used in aqueous formulations for subcutaneous injection insulin therapy.

In one aspect of the invention, N-terminally modified insulins according to the invention are useful as ultra-long acting insulins either as injection therapy in aqueous formulations or as oral therapy.

In one aspect the N-terminal modification of the N-terminally modified insulins according to the invention, in addition to conferring chemical stability towards aldehydes and/or ketones, may alter the insulin receptor affinity. For example, as described below, N-terminal modifications which at physiological pH render the N-terminals either neutral or negatively charged may confer a lower affinity for the insulin receptor.

A further aspect of this invention relates to furnishing of N-terminally modified insulins, such as acylated N-terminally modified insulins, which, when administered orally, have satisfactory bioavailabilities. Compared with the bioavailabilities of similar insulins without the N-terminal modification (parent insulins) given in similar doses, the bioavailability of preferred N-terminally modified insulins of this invention is similar. In one aspect the bioavailability is at least 10% higher than the bioavalilability of similar acylated insulins without the N-terminal modification given in similar doses, in one aspect the bioavailability is 20% higher, in one aspect the bioavailability is 25% higher, in one aspect the bioavailability is 30% higher, in one aspect the bioavailability is 35% higher, in one aspect the bioavailability is 40% higher, in one aspect the bioavailability is 45% higher, in one aspect the bioavailability is 50% higher, in one aspect the bioavailability is 55% higher, in one aspect the bioavailability is 60% higher, in one aspect the bioavailability is 65% higher, in one aspect the bioavailability is 70% higher, in one aspect the bioavailability is 80% higher, in one aspect the bioavailability is 90% higher, in one aspect the bioavailability is 100% higher, in one aspect the bioavailability is more than 100% higher than that of the parent insulins.

When used herein the term “parent insulin” shall mean a similar insulin without the N-terminal modification. For example if the N-terminally modified insulin is an acylated N-terminally modified insulin, then the parent insulin is an acylated insulin with the same peptide part and the same lipophilic substituent but without the N-terminal modification, or for example if the N-terminally modified insulin is an acylated, protease stabilised N-terminally modified insulin, then the parent insulin is an acylated, protease stabilised insulin with the same peptide part and the same lipophilic substituent but without N-terminal modification.

A further aspect of this invention relates to furnishing of N-terminally modified insulins which, when administered orally, have satisfactory bioavailabilities relative to when administered as i.v. administration. Bioavailabilities of preferred compounds of this invention (relative to i.v. administration) are at least 0.3%, in one aspect at least 0.5%, in one aspect at least 1%, in one aspect at least 1.5%, in one aspect at least 2%, in one aspect at least 2.5%, in one aspect at least 3%, in one aspect at least 3.5%, in one aspect at least 4%, in one aspect at least 5%, in one aspect at least 6%, in one aspect at least 7%, in one aspect at least 8%, in one aspect at least 9%, in one aspect at least 10% relative to the bioavailability when the N-terminally modified insulin is administered i.v.

A further aspect of this invention relates to furnishing of N-terminally modified insulins which, when administered orally, have satisfactory bioavailabilities relative to when administered as s.c. (subcutaneous) administration. Bioavailabilities of preferred compounds of this invention (relative to s.c. administration) are at least 0.3%, in one aspect at least 0.5%, in one aspect at least 1%, in one aspect at least 1.5%, in one aspect at least 2%, in one aspect at least 2.5%, in one aspect at least 3%, in one aspect at least 3.5%, in one aspect at least 4%, in one aspect at least 5%, in one aspect at least 6%, in one aspect at least 7%, in one aspect at least 8%, in one aspect at least 9%, in one aspect at least 10% relative to the bioavailability when the N-terminally modified insulin is administered s.c.

Standard assays for measuring insulin bioavailability are known to the person skilled in the art and include inter alia measurement of the relative areas under the curve (AUC) for the concentration of the insulin in question administered orally and intravenously (i.v.) in the same species. Quantitation of insulin concentrations in blood (plasma) samples can be done using for example antibody assays (ELISA) or by mass spectrometry.

A further aspect of this invention relates to furnishing of N-terminally modified insulins which have satisfactory potencies. Compared with the potency of human insulin, potencies of preferred N-terminally modified insulins of the invention may be at least 5%, in one aspect at least 10%, in one aspect at least 20%, in one aspect at least 30%, in one aspect at least 40%, in one aspect at least 50%, in one aspect at least 75% and in one aspect at least 100% of the potency of human insulin.

Apparent in vivo potency can be measured by comparison of blood glucose versus time profiles of the insulin in question with the comparator insulin given in similar doses. Other means to measure in vivo potency are given in the examples.

Standard assays for measuring insulin in vitro potency are known to the person skilled in the art and include inter alia (1) insulin radioreceptor assays, in which the relative potency of an insulin is defined as the ratio of insulin to insulin analogue required to displace 50% of ¹²⁵I-insulin specifically bound to insulin receptors present on cell membranes, e.g., a rat liver plasma membrane fraction; (2) lipogenesis assays, performed, e.g., with rat adipocytes, in which relative insulin potency is defined as the ratio of insulin to insulin analogue required to achieve 50% of the maximum conversion of [3-³H] glucose into organic-extractable material (i.e. lipids); (3) glucose oxidation assays in isolated fat cells in which the relative potency of the insulin analogue is defined as the ratio of insulin to insulin analogue to achieve 50% of the maximum conversion of glucose-1-[¹⁴C] into [¹⁴CO₂]; (4) insulin radioimmunoassays which can determine the immunogenicity of insulin analogues by measuring the effectiveness by which insulin or an insulin analogue competes with ¹²⁵I-insulin in binding to specific anti-insulin antibodies; and (5) other assays which measure the binding of insulin or an insulin analogue to antibodies in animal blood plasma samples, such as ELISA assays possessing specific insulin antibodies.

N-terminally modified insulins according to the invention may have a prolonged time-action profile, i.e. provide an insulin effect in hyperglycemic, e.g., diabetic, patients that lasts longer than human insulin. In other words, an insulin with a prolonged time-action profile has prolonged lowering of the glucose level compared to human insulin. In one aspect, the N-terminally modified insulin according to the invention provides an insulin effect for from about 8 hours to about 2 weeks after a single administration of the insulin molecule. In one aspect, the insulin effect lasts from about 24 hours to about 2 weeks. In one aspect, the effect lasts from about 24 hours to about 1 week. In a further aspect, the effect lasts from about 1 week to about 2 weeks. In yet a further aspect, the effect lasts about 1 week. In yet a further aspect, the effect lasts about 2 weeks. In one aspect, the effect lasts from about 1 day to about 7 days. In a further aspect, the effect lasts from about 7 days to about 14 days. In yet a further aspect, the effect lasts about 7 days. In yet a further aspect, the effect lasts about 14 days. In one aspect, the effect lasts from about 2 days to about 7 days. In yet a further aspect, the effect lasts about 3 days. In yet a further aspect, the effect lasts about 7 days.

In one aspect, the N-terminally modified insulin according to the invention provides an insulin effect for from about 8 hours to about 24 hours after a single administration of the insulin molecule. In one aspect, the insulin effect lasts from about 10 hours to about 24 hours. In one aspect, the effect lasts from about 12 hours to about 24 hours. In a further aspect, the effect lasts from about 16 hours to about 24 hours. In yet a further aspect, the effect lasts from about 20 hours to about 24 hours. In yet a further aspect, the effect lasts about 24 hours.

In one aspect, the insulin effect lasts from about 24 hours to about 96 hours. In one aspect, the insulin effect lasts from about 24 hours to about 48 hours. In one aspect, the insulin effect lasts from about 24 hours to about 36 hours. In one aspect, the insulin effect lasts from about 1 hour to about 96 hours. In one aspect, the insulin effect lasts from about 1 hour to about 48 hours. In one aspect, the insulin effect lasts from about 1 hour to about 36 hours.

Duration of action (time-action profile) can be measured by the time that blood glucose is suppressed, or by measuring relevant pharmacokinetic properties, for example t_{1/2} or MRT (mean residence time).

A further aspect of this invention relates to the furnishing of N-terminally modified insulins having a satisfactory prolonged action following oral administration relative to human insulin. Compared with human insulin, the duration of action of preferred N-terminally modified insulins of this invention is at least 10% longer. In one aspect the duration is at least 20% longer, in one aspect at least 25% longer, in one aspect at least 30% longer, in one aspect at least 35% longer, in one aspect at least 40% longer, in one aspect at least 45% longer, in one aspect at least 50% longer, in one aspect at least 55% longer, in one aspect at least 60% longer, in one aspect at least 65% longer, in one aspect at least 70% longer, in one aspect at least 80% longer, in one aspect at least 90% longer, in one aspect at least 100% longer, in one aspect more than 100% longer than that of human insulin.

In one aspect, compared with a once daily insulin such as LysB29(Nε-tetradecanoyl)desB30 human insulin or A21Gly, B31Arg, B32Arg human insulin, the duration of action of preferred N-terminally modified insulins of this invention is at least 10% longer. In one aspect the duration is at least 20% longer, in one aspect at least 25% longer, in one aspect at least 30% longer, in one aspect at least 35% longer, in one aspect at least 40% longer, in one aspect at least 45% longer, in one aspect at least 50% longer, in one aspect at least 55% longer, in one aspect at least 60% longer, in one aspect at least 65% longer, in one aspect at least 70% longer, in one aspect at least 80% longer, in one aspect at least 90% longer, in one aspect at least 100% longer, in one aspect more than 100% longer than that of a once daily insulin such as LysB29(Nε-tetradecanoyl)desB30 human insulin or A21Gly, B31Arg, B32Arg human insulin.

In one aspect, compared with a once daily insulin such as LysB29(Nε-tetradecanoyl)desB30 human insulin or A21Gly, B31Arg, B32Arg human insulin, the duration of action of preferred N-terminally modified insulins of this invention is at least 100% longer. In one aspect the duration is at least 200% longer, in one aspect at least 250% longer, in one aspect at least 300% longer, in one aspect at least 350% longer, in one aspect at least 400% longer, in one aspect at least 450% longer, in one aspect at least 500% longer, in one aspect at least 550% longer, in one aspect at least 600% longer, in one aspect at least 650% longer, in one aspect at least 700% longer, in one aspect at least 800% longer, in one aspect at least 900% longer, in one aspect at least 1000% longer, in one aspect more than 1000% longer than that of a once daily insulin such as LysB29(Nε-tetradecanoyl)desB30 human insulin or A21Gly, B31Arg, B32Arg human insulin.

N-terminal modification groups for use in the invention may be neutral or positively charged or negatively charged at physiological pH.

The charge of the N-terminal modification group of the N-terminally modified insulin may be chosen so that the N-terminally modified insulin has retained or altered affinity for the insulin receptor (IR) compared to the insulin receptor affinity of the parent insulin.

For example, an N-terminal modification group which at physiological pH (i.e. pH 7.4) is neutral or negatively charged may result in reduced IR affinity compared to the parent insulin without N-terminal modification. As another example, an N-terminal modification group which at physiological pH is positively charged may result in retained or only slightly reduced IR affinity compared to the parent insulin without N-terminal modification.

In one aspect of the invention, an N-terminally modified insulin is obtained, wherein the insulin is an acylated, protease stabilised insulin and the N-terminal modification is with positively charged N-terminal modification groups.

In a further aspect, the N-terminally modified insulin of the invention consists of a peptide part, a lipophilic substituent and an N-terminal modification group.

Herein, the term protease stabilised insulin means the insulin having an improved stability against degradation from proteases relative to human insulin.

An acylated, protease stabilised insulin is herein to be understood as an acylated insulin, which is subjected to slower degradation by one or more proteases relative to human insulin. In one embodiment a protease stabilised insulin according to the invention is subjected to slower degradation by one or more proteases relative to human insulin. In a further embodiment of the invention an insulin acylated, protease stabilised according to the invention is stabilized against degradation by one or more enzymes selected from the group consisting of: pepsin (such as e.g. the isoforms pepsin A, pepsin B, pepsin C and/or pepsin F), chymotrypsin (such as e.g. the isoforms chymotrypsin A, chymotrypsin B and/or chymotrypsin C), trypsin, Insulin-Degrading Enzyme (IDE), elastase (such as e.g. the isoforms pancreatic elastase I and/or II), carboxypeptidase (e.g. the isoforms carboxypeptidase A, carboxypeptidase A2 and/or carboxypeptidase B), aminopeptidase, cathepsin D and other enzymes present in intestinal extracts derived from rat, pig or human.

In one embodiment an acylated, protease stabilised insulin according to the invention is stabilized against degradation by one or more enzymes selected from the group consisting of: chymotrypsin, trypsin, Insulin-Degrading Enzyme (IDE), elastase, carboxypeptidases, aminopeptidases and cathepsin D. In a further embodiment an acylated, protease stabilised insulin according to the invention is stabilized against degradation by one or more enzymes selected from the group consisting of: chymotrypsin, carboxypeptidases and IDE. In a yet further embodiment an acylated, protease stabilised insulin according to the invention is stabilized against degradation by one or more enzymes selected from: chymotrypsin and carboxypeptidases.

By the term “positively charged at physiological pH” when used about the N-terminal modification group as herein described is meant, that in a solution comprising the N-terminally modified polypeptide at least 10% of the N-terminal modification groups have a charge of +1 at physiological pH. In one aspect at least 30% of the N-terminal modification groups in a solution of the N-terminally modified polypeptide have a charge of +1 at physiological pH. In a further aspect at least 50% of the N-terminal modification groups in a solution of the N-terminally modified polypeptide have a charge of +1 at physiological pH. In yet a further aspect at least 70% of the N-terminal modification groups in a solution of the N-terminally modified polypeptide have a charge of +1 at physiological pH. In still a further aspect at least 90% of the N-terminal modification groups in a solution of the N-terminally modified polypeptide have a charge of +1 at physiological pH.

Examples of positively charged N-terminal modification groups at physiological pH include but is not limited to: N,N-di-C1-4 alkyl such as N,N-dimethyl and N,N-diethyl, N-amidinyl, 4-(N,N-dimethylamino)butanoyl, 3-(1-piperidinyl)propionyl, 3-(N,N-dimethylamino)propionyl, N,N-dimethyl-glycyl, and N,N,N-trimethyl-glycyl:

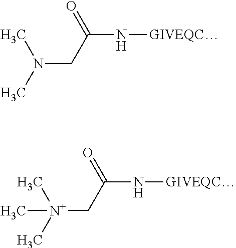

In one aspect of the invention an N-terminally modified insulin is obtained, wherein the insulin is fatty acid acylated, such as fatty diacid acylated, in a position other than a N-terminal position of the insulin and the N-terminal modification is with neutral or negatively charged N-terminal modification groups.

When used herein the term “neutral at physiological pH” when used about the N-terminal modification group as herein described is meant, that in a solution comprising the N-terminally modified insulin at least 10% of the N-terminal modification groups have a neutral charge (i.e. the charge is 0) at physiological pH. In one aspect at least 30% of the N-terminal modification groups in a solution of the N-terminally modified polypeptide have a neutral charge at physiological pH. In a further aspect at least 50% of the N-terminal modification groups in a solution of the N-terminally modified polypeptide have a neutral charge at physiological pH. In yet a further aspect at least 70% of the N-terminal modification groups in a solution of the N-terminally modified polypeptide have a neutral charge at physiological pH. In still a further aspect at least 90% of the N-terminal modification groups in a solution of the N-terminally modified polypeptide have a neutral charge at physiological pH.

Examples of neutral N-terminal modification groups at physiological pH include but is not limited to: Carbamoyl, thiocarbamoyl, and C1-4 chain acyl groups, such as formyl, acetyl, propionyl, butyryl, and pyroglutamyl:

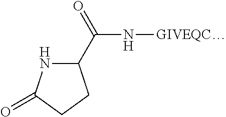

When used herein the term “negatively charged at physiological pH” when used about the N-terminal modification group as herein described is meant, that in a solution comprising the N-terminally modified insulin at least 10% of the N-terminal modification groups have a charge of −1 (i.e. minus 1) at physiological pH. In one aspect at least 30% of the N-terminal modification groups in a solution of the N-terminally modified polypeptide have a charge of −1 at physiological pH. In a further aspect at least 50% of the N-terminal modification groups in a solution of the N-terminally modified polypeptide have a charge of −1 at physiological pH. In yet a further aspect at least 70% of the N-terminal modification groups in a solution of the N-terminally modified polypeptide have a charge of −1 at physiological pH. In still a further aspect at least 90% of the N-terminal modification groups in a solution of the N-terminally modified polypeptide have a charge of −1 at physiological pH. Examples of negatively charged N-terminal modification groups at physiological pH include but is not limited to: oxalyl, glutaryl, diglycolyl (other names: 3-oxoglutaryl and carboxymethoxyacetyl).

In one aspect, a negatively charged N-terminal modification group at physiological pH according to the invention is not malonyl or succinyl. In one aspect, a negatively charged N-terminal modification group at physiological pH according to the invention is not malonyl. In one aspect, a negatively charged N-terminal modification group at physiological pH according to the invention is not succinyl.

In one aspect of the invention an insulin is obtained which is N-terminally modified and furthermore substituted with a lipophilic substituent in a position other than one of the N-terminals of the insulin, wherein the lipophilic substituent consists of a fatty acid or a difatty acid attached to the insulin optionally via a linker. The linker may be any suitable portion inbetween the fatty acid or the fatty diacid and the point of attachment to the insulin, which portion may also be referred to as a linker moiety, spacer, or the like.

In one aspect, a linker is present and comprises one or more entities selected from the group consisting of: Gly, D-Ala, L-Ala, D-αGlu, L-αGlu, D-γGlu, L-γGlu, D-αAsp, L-αAsp, D-βAsp, L-βAsp, βAla, 4-aminobutyric acid, 5-aminovaleric acid, 6-aminohexanoic acid, D-GIu-α-amide, L-Glu-α-amide, D-Glu-γ-amide, L-Glu-γ-amide, D-Asp-α-amide, L-Asp-α-amide, D-Asp-β-amide, L-Asp-β-amide, or:

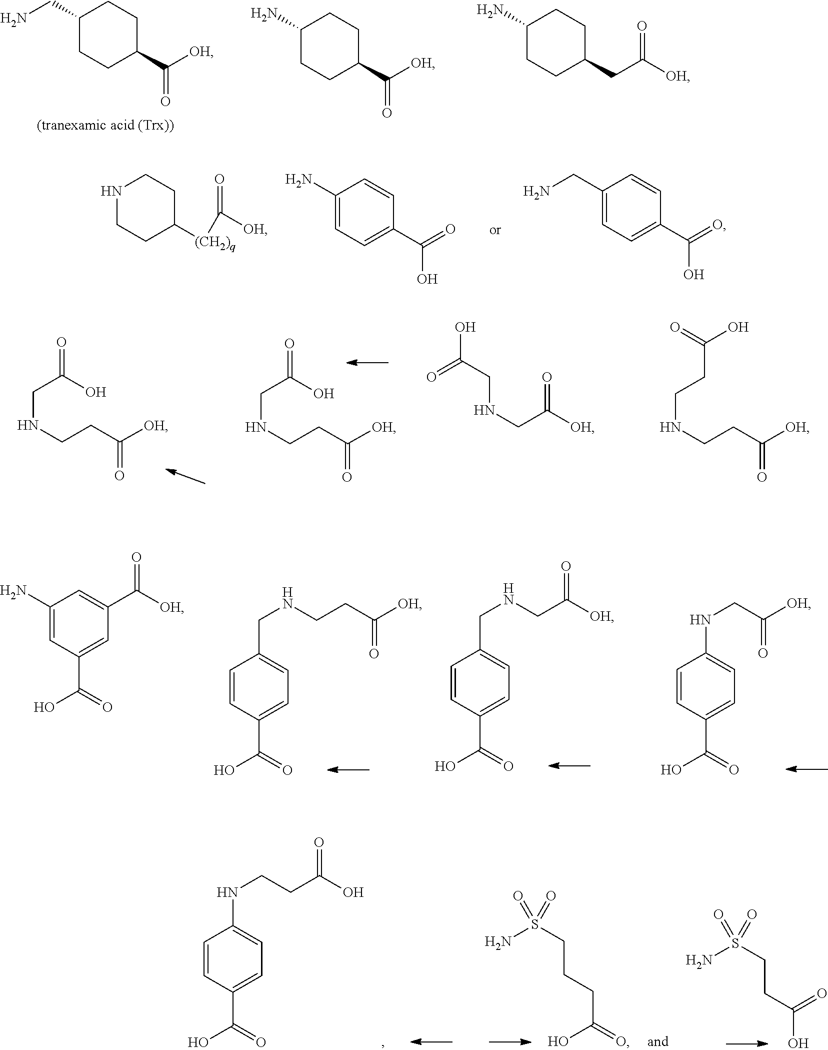

from which a hydrogen atom and/or a hydroxyl group has been removed, wherein q is 0, 1, 2, 3 or 4 and, in this embodiment may, alternatively, be 7-aminoheptanoic acid or 8-aminooctanoic acid and wherein the arrows indicate the attachment point to, or if more linkers are present, towards the amino group of the protease stabilised insulin.

In one aspect, a linker is present and comprises gamma-Glu (γGlu) entities, one or more OEG entities or a combination thereof.

Herein, the term “fatty acid” covers a linear or branched, aliphatic carboxylic acids having at least two carbon atoms and being saturated or unsaturated. Non limiting examples of fatty acids are myristic acid, palmitic acid, and stearic acid.

Herein, the term “fatty diacid” covers a linear or branched, aliphatic dicarboxylic acids having at least two carbon atoms and being saturated or unsaturated. Non limiting examples of fatty diacids are hexanedioic acid, octanedioic acid, decanedioic acid, dodecanedioic acid, tetradecanedioic acid, hexadecanedioic acid; heptadecanedioic acid, octadecanedioic acid, and eicosanedioic acid.

Oral pharmaceutical compositions comprising N-terminally modified insulins are also contemplated by the invention. In one aspect an oral pharmaceutical composition is a composition comprising one or more lipids and an N-terminally modified insulin.

N-terminally modified insulins of the invention are surprisingly chemically stable when used in lipid pharmaceutical formulations. In one aspect, a lipid pharmaceutical formulation comprising an N-terminal modified insulin according to the invention is chemically stable for at least 2 weeks of usage and 1 year of storage. In one aspect, a lipid pharmaceutical formulation comprising an N-terminal modified insulin according to the invention is chemically stable for at least 4 weeks of usage and 1 year of storage. In one aspect, a lipid pharmaceutical formulation comprising an N-terminal modified insulin according to the invention is chemically stable for at least 4 weeks of usage and 2 years of storage. In one aspect, a lipid pharmaceutical formulation comprising an N-terminal modified insulin according to the invention is chemically stable for at least 6 weeks of usage and 2 years of storage.

It is known to the person skilled in the art that a common method for stabilizing insulins in aqueous pharmaceutical formulations is to add zinc to the pharmaceutical formulation and thereby form insulin hexamers with the zinc. In one aspect of the invention, a pharmaceutical lipid composition comprising an N-terminally modified insulin and no zinc or only trace amounts of zinc is chemically stable similar to an aqueous pharmaceutical formulation comprising the N-terminal modified insulin and zinc.

It has surprisingly been found that non-aqueous liquid insulin pharmaceutical compositions comprising a N-terminally modified insulin, one or more lipids and optionally one or more surfactants are chemically stable. In one aspect the pharmaceutical composition of the invention comprises a N-terminally modified insulin, one or more lipids, one or more surfactants and a cosolvent. In one aspect of the invention the cosolvent is propylene glycol.

In one aspect of the invention, the a N-terminally modified insulin is present in the pharmaceutical composition in a concentration between from 0.1 to 30% (w/w) of the total amount of ingredients in the composition. In another aspect the insulin is present in a concentration between from 0.5 to 20% (w/w). In another aspect the insulin is present in a concentration between from 1 to 10% (w/w).

In one aspect of the invention, the N-terminally modified insulin is present in the pharmaceutical composition in a concentration between from 0.2 mM to 100 mM. In another aspect the a N-terminally modified insulin is present in a concentration between from 0.5 to 70 mM. In another aspect the a N-terminally modified insulin is present in a concentration between from 0.5 to 35 mM. In another aspect the a N-terminally modified insulin is present in a concentration between from 1 to 30 mM.

When used herein the term “lipid” The term “lipid” is herein used for a substance, material or ingredient that is more mixable with oil than with water. A lipid is insoluble or almost insoluble in water but is easily soluble in oil or other nonpolar solvents.

The term “lipid” can comprise one or more lipophilic substances, i.e. substances that form homogeneous mixtures with oils and not with water. Multiple lipids may constitute the lipophilic phase of the non-aqueous liquid pharmaceutical composition and form the oil aspect. At room temperature, the lipid can be solid, semisolid or liquid. For example, a solid lipid can exist as a paste, granular form, powder or flake. If more than one excipient comprises the lipid, the lipid can be a mixture of liquids, solids, or both.

Examples of solid lipids i.e., lipids which are solid or semisolid at room temperature, include, but are not limited to, the following:

1. Mixtures of mono-, di- and triglycerides, such as hydrogenated coco-glycerides (melting point (m.p.) of about 33.5° C. to about 37° C.], commercially-available as WITEPSOL H15 from Sasol Germany (Witten, Germany); Examples of fatty acid triglycerides e.g., C10-C22 fatty acid triglycerides include natural and hydrogenated oils, such as vegetable oils;

2. Esters, such as propylene glycol (PG) stearate, commercially available as MONOSTEOL (m.p. of about 33° C. to about 36° C.) from Gattefosse Corp. (Paramus, N.J.); diethylene glycol palmito stearate, commercially available as HYDRINE (m.p. of about 44.5° C. to about 48.5° C.) from Gattefosse Corp.;

3. Polyglycosylated saturated glycerides, such as hydrogenated palm/palm kernel oil PEG-6 esters (m.p. of about 30.5° C. to about 38° C.), commercially-available as LABRAFIL M2130 CS from Gattefosse Corp. or Gelucire 33/01;

4. Fatty alcohols, such as myristyl alcohol (m.p. of about 39° C.), commercially available as LANETTE 14 from Cognis Corp. (Cincinnati, Ohio); esters of fatty acids with fatty alcohols, e.g., cetyl palmitate (m.p. of about 50° C.); isosorbid monolaurate, e.g. commercially available under the trade name ARLAMOL ISML from Uniqema (New Castle, Del.), e.g. having a melting point of about 43° C.;

5. PEG-fatty alcohol ether, including polyoxyethylene (2) cetyl ether, e.g. commercially available as BRIJ 52 from Uniqema, having a melting point of about 33° C., or polyoxyethylene (2) stearyl ether, e.g. commercially available as BRIJ 72 from Uniqema having a melting point of about 43° C.;

6. Sorbitan esters, e.g. sorbitan fatty acid esters, e.g. sorbitan monopalmitate or sorbitan monostearate, e.g., commercially available as SPAN 40 or SPAN 60 from Uniqema and having melting points of about 43° C. to 48° C. or about 53° C. to 57° C. and 41° C. to 54° C., respectively; and

7. Glyceryl mono-C6-C14-fatty acid esters. These are obtained by esterifying glycerol with vegetable oil followed by molecular distillation. Monoglycerides include, but are not limited to, both symmetric (i.e. β-monoglycerides) as well as asymmetric monoglycerides α-monoglycerides). They also include both uniform glycerides (in which the fatty acid constituent is composed primarily of a single fatty acid) as well as mixed glycerides (i.e. in which the fatty acid constituent is composed of various fatty acids). The fatty acid constituent may include both saturated and unsaturated fatty acids having a chain length of from e.g. C8-C14. Particularly suitable are glyceryl mono laurate e.g. commercially available as IMWITOR 312 from Sasol North America (Houston, Tex.), (m.p. of about 56° C.-60° C.); glyceryl mono dicocoate, commercially available as IMWITOR 928 from Sasol (m.p. of about 33° C.-37° C.); monoglyceryl citrate, commercially available as IMWITOR 370, (m.p. of about 59 to about 63° C.); or glyceryl mono stearate, e.g., commercially available as IMWITOR 900 from Sasol (m.p. of about 56° C.-61° C.); or self-emulsifying glycerol mono stearate, e.g., commercially available as IMWITOR 960 from Sasol (m.p. of about 56° C.-61° C.).

Examples of liquid and semisolid lipids, i.e., lipids which are liquid or semisolid at room temperature include, but are not limited to, the following:

1. Mixtures of mono-, di- and triglycerides, such as medium chain mono- and diglycerides, glyceryl caprylate/caprate, commercially-available as CAPMUL MCM from Abitec Corp. (Columbus, Ohio); and glycerol monocaprylate, commercially available as RYLO MG08 Pharma and glycerol monocaprate, commercially available as RYLO MG10 Pharma from DANISCO.

2. Glyceryl mono- or di fatty acid ester, e.g. of C6-C18, e.g. C6-C16 e.g. C8-C10, e.g. C8, fatty acids, or acetylated derivatives thereof, e.g. MYVACET 9-45 or 9-08 from Eastman Chemicals (Kingsport, Tenn.) or IMWITOR 308 or 312 from Sasol;

3. Propylene glycol mono- or di-fatty acid ester, e.g. of C8-C20, e.g. C8-C12, fatty acids, e.g. LAUROGLYCOL 90, SEFSOL 218, or CAPRYOL 90 or CAPMUL PG-8 (same as propylene glycol caprylate) from Abitec Corp. or Gattefosse;

4. Oils, such as safflower oil, sesame oil, almond oil, peanut oil, palm oil, wheat germ oil, corn oil, castor oil, coconut oil, cotton seed oil, soybean oil, olive oil and mineral oil;

5. Fatty acids or alcohols, e.g. C8-C20, saturated or mono- or di-unsaturated, e.g. oleic acid, oleyl alcohol, linoleic acid, capric acid, caprylic acid, caproic acid, tetradecanol, dodecanol, decanol;

6. Medium chain fatty acid triglycerides, e.g. C8-C12, e.g. MIGLYOL 812, or long chain fatty acid triglycerides, e.g. vegetable oils;

7. Transesterified ethoxylated vegetable oils, e.g. commercially available as LABRAFIL M2125 CS from Gattefosse Corp;

8. Esterified compounds of fatty acid and primary alcohol, e.g. C8-C20, fatty acids and C2-C3 alcohols, e.g. ethyl linoleate, e.g. commercially available as NIKKOL VF-E from Nikko Chemicals (Tokyo, Japan), ethyl butyrate, ethyl caprylate oleic acid, ethyl oleate, isopropyl myristate and ethyl caprylate;

9. Essential oils, or any of a class of volatile oils that give plants their characteristic odours, such as spearmint oil, clove oil, lemon oil and peppermint oil;

10. Fractions or constituents of essential oils, such as menthol, carvacrol and thymol;

11. Synthetic oils, such as triacetin, tributyrin;

12. Triethyl citrate, acetyl triethyl citrate, tributyl citrate, acetyl tributyl citrate;

13. Polyglycerol fatty acid esters, e.g. diglyceryl monooleate, e.g. DGMO-C, DGMO-90, DGDO from Nikko Chemicals; and

14. Sorbitan esters, e.g. sorbitan fatty acid esters, e.g. sorbitan monolaurate, e.g. commercially available as SPAN 20 from Uniqema.

15. Phospholipids, Alkyl-O-Phospholipids, Diacyl Phosphatidic Acids, Diacyl

Phosphatidyl Cholines, Diacyl Phosphatidyl Ethanolamines, Diacyl Phosphatidyl Glycerols, Di-O-Alkyl Phosphatidic Acids, L-alpha-Lysophosphatidylcholines (LPC), L-alpha-Lysophosphatidylethanolamines (LPE), L-alpha-Lysophosphatidylglycerol (LPG), L-alpha-Lysophosphatidylinositols (LPI), L-alpha-Phosphatidic acids (PA), L-alpha-Phosphatidylcholines (PC), L-alpha-Phosphatidylethanolamines (PE), L-alpha-Phosphatidylglycerols (PG), Cardiolipin (CL), L-alpha-Phosphatidylinositols (PI), L-alpha-Phosphatidylserines (PS), Lyso-Phosphatidylcholines, Lyso-Phosphatidylglycerols, sn-Glycerophosphorylcholines commercially available from LARODAN, or soybean phospholipid (Lipoid S100) commercially available from Lipoid GmbH.

16. Polyglycerol fatty acid esters, such as polyglycerol oleate (Plurol Oleique from Gattefosse).

In one aspect of the invention, the lipid is one or more selected from the group consisting of mono-, di-, and triglycerides. In a further aspect, the lipid is one or more selected from the group consisting of mono- and diglycerides. In yet a further aspect, the lipid is Capmul MCM or Capmul PG-8. In a still further aspect, the lipid is Capmul PG-8. In a further aspect the lipid is Glycerol monocaprylate (Rylo MG08 Pharma from Danisco).

In one aspect the lipid is selected from the group consisting of: Glycerol mono-caprylate (such as e.g. Rylo MG08 Pharma) and Glycerol mono-caprate (such as e.g. Rylo MG10 Pharma from Danisco). In another aspect the lipid is selected from the group consisting of: propyleneglycol caprylate (such as e.g. Capmul PG8 from Abitec or Capryol PGMC, or Capryol 90 from Gattefosse).

In one aspect of the invention, the lipid is present in the pharmaceutical composition in a concentration between from 10% to 90% (w/w) of the total amount of ingredients including insulin in the composition. In another aspect the lipid is present in a concentration between from 10 to 80% (w/w). In another aspect the lipid is present in a concentration between from 10 to 60% (w/w). In another aspect the lipid is present in a concentration between from 15 to 50% (w/w). In another aspect the lipid is present in a concentration between from 15 to 40% (w/w). In another aspect the lipid is present in a concentration between from 20 to 30% (w/w). In another aspect the lipid is present in a concentration of about 25% (w/w).

In one aspect of the invention, the lipid is present in the pharmaceutical composition in a concentration between from 100 mg/g to 900 mg/g of the total amount of ingredients including insulin in the composition. In another aspect the lipid is present in a concentration between from 100 to 800 mg/g. In another aspect the lipid is present in a concentration between from 100 to 600 mg/g. In another aspect the lipid is present in a concentration between from 150 to 500 mg/g. In another aspect the lipid is present in a concentration between from 150 to 400 mg/g. In another aspect the lipid is present in a concentration between from 200 to 300 mg/g. In another aspect the lipid is present in a concentration of about 250 mg/g.

In one aspect of the invention, the cosolvent is present in the pharmaceutical composition in a concentration between from 0% to 30% (w/w) of the total amount of ingredients including insulin in the composition. In another aspect the cosolvent is present in a concentration between from 5% to 30% (w/w). In another aspect the cosolvent is present in a concentration between from 10 to 20% (w/w).

In one aspect of the invention, the cosolvent is present in the pharmaceutical composition in a concentration between from 0 mg/g to 300 mg/g of the total amount of ingredients including insulin in the composition. In another aspect the cosolvent is present in a concentration between from 50 mg/g to 300 mg/g. In another aspect the cosolvent is present in a concentration between from 100 to 200 mg/g.

In one aspect of the invention the oral pharmaceutical composition does not contain oil or any other lipid component or surfactant with an HLB below 7. In a further aspect the composition does not contain oil or any other lipid component or surfactant with an HLB below 8. In a yet further aspect the composition does not contain oil or any other lipid component or surfactant with an HLB below 9. In a yet further aspect the composition does not contain oil or any other lipid component or surfactant with an HLB below 10.

The hydrophilic-lipophilic balance (HLB) of each of the non-ionic surfactants of the liquid non-aqueous pharmaceutical composition of the invention is above 10 whereby high insulin peptide (such as insulin derivative) drug loading capacity and high oral bioavailability are achieved. In one aspect the non-ionic surfactants according to the invention are non-ionic surfactants with HLB above 11. In one aspect the non-ionic surfactants according to the invention are non-ionic surfactants with HLB above 12.

The term “about” as used herein means in reasonable vicinity of the stated numerical value, such as plus or minus 10%.

A non-limiting example of lipid pharmaceutical compositions may e.g. be found in the patent applications WO 08/145,728, WO 2010/060667 and WO 2011/086093.

In one aspect, an N-terminally modified insulin of the invention is selected from the group consisting of:

A1(N^{α},N^{α}-Dimethyl), A14E, B1(N^{α},N^{α}-dimethyl), B25H, B29K(N^{ε}octadecanedioyl-gGlu-2xOEG), desB30 human insulin

A1(N^{α},N^{α}-Diethyl), A14E, B1(N^{α},N^{α}-diethyl), B25H, B29K(N^{ε}Octadecanedioyl-gGlu2xOEG), desB30 human insulin

A1(N^{α},N^{α}-Dimethyl), A14E, B1(N^{α},N^{α}-dimethyl), B16H, B25H, B29K(N^{ε}hexadecanedioyl-gGlu), desB30 human insulin

A1(N^{α},N^{α}-Dimethyl), A14E, B1(N^{α},N^{α}-dimethyl), B25H, desB27, B29K(N^{ε}octadecanedioyl-gGlu), desB30 human insulin

A1(N^{α},N^{α}-Dimethyl), A14E, B1(N^{α},N^{α}-dimethyl), B25H, desB27, B29K(N^{ε}octadecanedioyl-gGlu-2xOEG), desB30 human insulin

A1(N^{α},N^{α}-Dimethyl), A14E, B1(N^{α},N^{α}-dimethyl), desB27, B29K(N^{ε}octadecanedioyl-gGlu-2xOEG), desB30 human insulin

A1(N^{α},N^{α}-Dimethyl), A14E, B1(N^{α},N^{α}-dimethyl), B16H, B25H, B29K(N^{ε}eicosanedioyl-gGlu-2xOEG), desB30 human insulin

A1G(N^{α},N^{α}-Dimethyl), A14E, B1F(N^{α},N^{α}-dimethyl), B25H, desB27, B29K(N^{ε}hexadecanedioyl-gGlu), desB30 human insulin

A1G(N^{α},N^{α}-Dimethyl), A14E, B1F(N(alpha),N(N^{α},N^{α}-dimethyl), B25H, desB27, B29K(N^{ε}hexadecanedioyl-gGlu-2xOEG), desB30 human insulin

A1(N^{α},N^{α}-Dimethyl), A14E, B1(N^{α},N^{α}-dimethyl), desB27, B29K(N^{ε}octadecanedioyl-gGlu), desB30 human insulin

A1(N^{α},N^{α}-Dimethyl), A14E, B1(N^{α},N^{α}-dimethyl), B25H, B29K(N^{ε}octadecanedioyl-gGlu), desB30 human insulin

A1(N^{α}Carbamoyl), A14E, B1(N^{α}Carbamoyl), B25H, B29K(N^{ε}octadecanedioyl-gGlu2xOEG), desB30 human insulin

A1(N^{α}Carbamoyl), A14E, B1(N^{α}Carbamoyl), B25H, B29K(N^{ε}hexadecanedioyl-gGlu), desB30 human insulin

A1(N^{α}Carbamoyl), A14E, B1(N^{α}Carbamoyl), B25H, B29K(N^{ε}eicosanedioyl-gGlu), desB30 human insulin

A1(N^{α}Carbamoyl), A14E, B1(N^{α}Carbamoyl), B25H, B29K(N^{ε}eicosanedioyl-gGlu-2xOEG), desB30 human insulin

A1(N^{α}Carbamoyl), A14E, B1(N^{α}Carbamoyl), B16H, B25H, B29K(N^{ε}eicosanedioyl-gGlu-2xOEG), desB30 human insulin

A1(N^{α}Carbamoyl), A14E, B1(N^{α}Carbamoyl), B25H, desB27, B29K(N^{ε}octadecandioyl-gGlu), desB30 human insulin

A1(N^{α}Carbamoyl), A14E, B1(N^{α}Carbamoyl), B25H, desB27, B29K(N^{ε}octadecandioyl-gGlu-2xOEG), desB30 human insulin

A1G(N(alpha)carbamoyl), A14E, B1F(N(alpha)carbamoyl), desB27, B29K(N(eps)hexadecanedioyl-gGlu), desB30 human insulin

A1G(N(alpha)carbamoyl), A14E, B1F(N(alpha)carbamoyl), desB27, B29K(Neps)-hexadecanedioyl-gGlu-2xOEG), desB30 human insulin

A1G(N(alpha)carbamoyl), A14E, B1F(N(alpha)carbamoyl), desB27, B29K(Neps)-eicosanedioyl-gGlu), desB30 human insulin

A1G(N^{α}carbamoyl), A14E, B1F(N^{α}carbamoyl), 816H, desB27, B29K(Neps)-eicosanedioyl-gGlu-2xOEG), desB30 human insulin

A1(N^{α}Carbamoyl), A14E, B1(N^{α}Carbamoyl), desB27, B29K(N^{ε}octadecanedioyl-gGlu), desB30 human insulin

A1(N^{α}Carbamoyl), A14E, B1(N^{α}Carbamoyl), B16H, B25H, B29K(N^{ε}eicosanedioyl-gGlu), desB30 human insulin

A1(N^{α}Carbamoyl), A14E, B1(N^{α}Carbamoyl), desB27, B29K(N^{ε}octadecanedioyl-gGlu-2xOEG), desB30 human insulin

A1(N^{α}Carbamoyl), A14E, B1(N^{α}carbamoyl), B25H, B29K(N^{ε}octadecanedioyl-gGlu), desB30 human insulin

A1(N^{α}Carbamoyl), A14E, B1(N^{ε}carbamoyl), B16H, B25H, B29K(N^{ε}eicosanedioyl-gGlu), desB30 human insulin

A1G(N^{α}carbamoyl), A14E, B1F(N^{α}carbamoyl), B25H, desB27, B29K(N^{ε}eicosanedioyl-gGlu-2xOEG), desB30 human insulin

A1G(N^{α}carbamoyl), A14E, B1F(N^{α}carbamoyl), desB27, B29K(N^{ε}eicosanedioyl-gGlu-2xOEG), desB30 human insulin

A1G(N^{α}carbamoyl), A14E, B1F(N^{α}carbamoyl), B16H, desB27, B29K(N^{ε}eicosanedioyl-gGlu-2xOEG), desB30 human insulin

A1G(N^{α}thiocarbamoyl), A14E, B1F(N N^{α}thiocarbamoyl), B25H, desB27, B29K(N^{ε}-octadecanedioyl-gGlu-2xOEG), desB30 human insulin

A1(N^{α}Acetyl), A14E, B1(N^{α}Acetyl), B25H, B29K(N^{ε}hexadecanedioyl-gGlu), desB30 human insulin

A1(N^{α}Acetyl), A14E, B1(N^{α}Acetyl), B25H, desB27, B29K(N^{ε}octadecanedioyl-gGlu), desB30 human insulin

A1(N^{α}Acetyl), A14E, B1(N^{α}Acetyl), B25H, B29K(N^{ε}octadecandioyl-gGlu-2xOEG), desB30 human insulin

A1(N^{α}Dimethylglycyl), A14E, B1(N^{α}Dimethylglycyl), B25H, B29K(N^{ε}octadecanedioyl-gGlu-2xOEG), desB30 human insulin

A1(N^{α}3-(N,N-Dimethylamino)propionyl), A14E, B1(N^{α}3-(N,N-dimethylamino)propionyl), B25H, B29K(N^{ε}-octadecanedioyl-gGlu-2xOEG), desB30 human insulin

A1(N^{α}4-(N,N-Dimethylamino)butanoyl), A14E, B1(N^{α}4-(N,N-dimethylamino)butanoyl), B25H, B29K(N^{ε}octadecanedioyl-gGlu-2xOEG), desB30 human insulin

A1(N^{α}3-(1-Piperidinyl)propionyl), A14E, B1(N^{α}3-(1-piperidinyl)propionyl), B25H, B29K(N^{ε}octadecanedioyl-gGlu-2xOEG), desB30 human insulin

A1(N^{α}Dimethylglycyl), A14E, B1(N^{α}Dimethylglycyl), B25H, desB27, B29K(N^{ε}octadecanedioyl-gGlu), desB30 human insulin

A1G(N^{α}acetyl), A14E, B1F(N^{α}acetyl), B25H, desB27, B29K(N^{ε}octadecanedioyl-gGlu-2xOEG), desB30 human insulin

A1G(N^{α}2-Picolyl), A14E, B1F(N^{α}2-Picolyl), B25H, desB27, B29K(N(eps)octadecanedioyl-gGlu-2xOEG), desB30 human insulin

A1(N^{α}Acetyl), A14E, B1(N^{α}Acetyl), B25H, B29K(N^{ε}eicosanedioyl-gGlu), desB30 human insulin

A1(N^{α}Acetyl), A14E, B1(N^{α}Acetyl), B25H, B29K(N^{ε}eicosanedioyl-gGlu-2xOEG), desB30 human insulin

A1(N^{α}Acetyl), A14E, B1(N^{α}Acetyl), B16H, B25H, B29K(N^{ε}eicosanedioyl-gGlu-2xOEG), desB30 human insulin

A1(N^{α}Acetyl), A14E, B1(N^{α}Acetyl), B16H, B25H, B29K(N^{ε}eicosanedioyl-gGlu), desB30 human insulin

A1(N^{α}Dimethylglycyl), A14E, B1(N^{α}Dimethylglycyl), B16H, B25H, B29K(N^{ε}hexadecanedioyl-gGlu), desB30 human insulin

A-1(N^{α}Trimethyl), A14E, B-1(N^{α}Trimethyl), B25H, B29K(N^{ε}octadecanedioyl-gGlu-2xOEG), desB30 human insulin

A1(N^{α}Acetyl), A14E, B1(N^{α}Acetyl), desB27, B29K(N^{ε}octadecanedioyl-gGlu), desB30 human insulin

A1(N^{α}Acetyl), A14E, B1(N^{α}Acetyl), desB27, B29K(N^{ε}octadecanedioyl-gGlu-2xOEG), desB30 human insulin

A1(N^{α}Acetyl), A14E, B1(N^{α}Acetyl), B25H, B29K(N^{ε}octadecanedioyl-gGlu), desB30 human insulin

A1G(N^{α}Acetyl), A14E, B1F(N^{α}Acetyl), desB27, B29K(N^{ε}eicosanedioyl-gGlu), desB30 human insulin

A1G(N^{α}Acetyl), A14E, B1F(N^{α}Acetyl), desB27, B29K(N^{ε}eicosanedioyl-gGlu-2xOEG), desB30 human insulin

A1G(N^{α}Acetyl), A14E, B1F(N^{α}Acetyl), B25H, desB27, B29K(N^{ε}eicosanedioyl-gGlu-2xOEG), desB30 human insulin

A1(N^{α}Succinyl), A14E, B1(N^{α}succinyl), B25H, desB27, B29K(N^{ε}octadecanedioyl-gGlu-2xOEG), desB30 human insulin

A1(N^{α}Succinyl), A14E, B1(N^{α}succinyl), B25H, B29K(N^{ε}octadecanedioyl-gGlu-2xOEG), desB30 human insulin

A1(N^{α}Succinyl), A14E, B1(N^{α}succinyl), desB27, B29K(N^{ε}-octadecanedioyl-gGlu-2xOEG), desB30 human insulin

A1(N^{α}Glutaryl), A14E, B1(N^{α}glutaryl), B25H, B29K(N^{ε}octadecanedioyl-gGlu-2xOEG), desB30 human insulin

A1(N^{α}Glutaryl), A14E, B1(N^{α}glutaryl), desB27, B29K(N^{ε}octadecanedioyl-gGlu-2xOEG), desB30 human insulin

A1(N^{α}Diglycolyl), A14E, B1 (N^{α}diglycolyl), B25H, desB27, B29K(N^{ε}octadecanedioyl-gGlu-2xOEG), desB30 human insulin

A1(N^{α}Glutaryl), A14E, B1(N^{α}glutaryl), B25H, desB27, B29K(N^{ε}octadecanedioyl-gGlu-2xOEG), desB30 human insulin

A1(N^{α}Succinyl), A14E, B1(N^{α}succinyl), desB27, B29K(N^{ε}octadecanedioyl-gGlu), desB30 human insulin

A1(N^{α}Succinyl), A14E, B1(N^{α}succinyl), B25H, desB27, B29K(N^{ε}eicosanedioyl-gGlu-2xOEG), desB30 human insulin

A1(N^{α}Succinyl), A14E, B1(N^{α}succinyl), desB27, B29K(N^{ε}eicosanedioyl-gGlu-2xOEG), desB30 human insulin

A1(N^{α}Succinyl), A14E, B1(N^{α}succinyl), B16H, desB27, B29K(N^{ε}eicosanedioyl-gGlu-2xOEG), desB30 human insulin A1(N^{α}Succinyl), A14E, B1(N^{α}succinyl), B25H, B29K(N^{ε}eicosanedioyl-gGlu-2xOEG), desB30 human insulin

A1(N^{α}Succinyl), A14E, B1(N^{α}succinyl), desB27, B29K(N^{ε}eicosanedioyl-gGlu), desB30 human insulin

A1(N^{α}Glutaryl), A14E, B1(N^{α}glutaryl), desB27, B29K(N^{ε}eicosanedioyl-gGlu), desB30 human insulin

A1(N^{α}Glutaryl), A14E, B1(N^{α}glutaryl), desB27, B29K(N^{ε}eicosanedioyl-gGlu-2xOEG), desB30 human insulin

A1(N^{α}Glutaryl), A14E, B1(N^{α}glutaryl), B25H, desB27, B29K(N^{ε}eicosanedioyl-gGlu-2xOEG), desB30 human insulin

A1(N^{α}Glutaryl), A14E, B1(N^{α}glutaryl), desB27, B29K(N^{ε}eicosanedioyl-gGlu-2xOEG), desB30 human insulin

A1(N^{α}Glutaryl), A14E, B1(N^{α}glutaryl), B25H, B29K(N^{ε}eicosanedioyl-gGlu-2xOEG), desB30 human insulin

In one embodiment, an N-terminally modified insulin according to the invention has a peptide part which is selected from the group consisting of the following insulin peptides (i.e. insulins of the invention without N-terminal modifications and without the “lipophilic substituent” or acyl moiety): A14E, B25H, desB30 human insulin; A14H, B25H, desB30 human insulin; A14E, B1E, B25H, desB30 human insulin; A14E, B16E, B25H, desB30 human insulin; A14E, B25H, B28D, desB30 human insulin; A14E, B25H, B27E, desB30 human insulin; A14E, B1E, B25H, B27E, desB30 human insulin; A14E, B1E, B16E, B25H, B27E, desB30 human insulin; A8H, A14E, B25H, desB30 human insulin; A8H, A14E, B25H, B27E, desB30 human insulin; A8H, A14E, B1E, B25H, desB30 human insulin; A8H, A14E, B1E, B25H, B27E, desB30 human insulin; A8H, A14E, B1E, B16E, B25H, B27E, desB30 human insulin; A8H, A14E, B16E, B25H, desB30 human insulin; A14E, B25H, B26D, desB30 human insulin; A14E, B1E, B27E, desB30 human insulin; A14E, B27E, desB30 human insulin; A14E, B28D, desB30 human insulin; A14E, B28E, desB30 human insulin; A14E, B1E, B28E, desB30 human insulin; A14E, B1E, B27E, B28E, desB30 human insulin; A14E, B1E, B25H, B28E, desB30 human insulin; A14E, B1E, B25H, B27E, B28E, desB30 human insulin; A14D, B25H, desB30 human insulin; B25N, B27E, desB30 human insulin; A8H, B25N, B27E, desB30 human insulin; A14E, B27E, B28E, desB30 human insulin; A14E, B25H, B28E, desB30 human insulin; B25H, B27E, desB30 human insulin; B1E, B25H, B27E, desb30 human insulin; A8H, B1E, B25H, B27E, desB30 human insulin; A8H, B25H, B27E, desB30 human insulin; B25N, B27D, desB30 human insulin; A8H, B25N, B27D, desB30 human insulin; B25H, B27D, desB309 human insulin; A8H, B25H, B27D, desB30 human insulin; A(−1)P, A(O)P, A14E, B25H, desB30 human insulin; A14E, B(−1)P, B(O)P, B25H, desB30 human insulin; A(−1)P, A(O)P, A14E, B(−1)P, B(O)P, B25H, desB30 human insulin; A14E, B25H, B30T, B31L, B32E human insulin; A14E, B25H human insulin; A14E, B16H, B25H, desB30 human insulin; A14E, B10P, B25H, desB30 human insulin; A14E, B10E, B25H, desB30 human insulin; A14E, B4E, B25H, desB30 human insulin; A14H, B16H, B25H, desB30 human insulin; A14H, B10E, B25H, desB30 human insulin; A13H, A14E, B10E, B25H, desB30 human insulin; A13H, A14E, B25H, desB30 human insulin; A14E, A18Q, B3Q, B25H, desB30 human insulin; A14E, B24H, B25H, desB30 human insulin; A14E, B25H, B26G, B27G, B28G, desB30 human insulin; A14E, A21G, B25H, B26G, B27G, B28G, desB30 human insulin; A14E, A18Q, A21Q, B3Q, B25H, desB30 human insulin; A14E, A18Q, A21Q, B3Q, B25H, B27E, desB30 human insulin; A14E, A18Q, B3Q, B25H, desB30 human insulin; A13H, A14E, B1E, B25H, desB30 human insulin; A13N, A14E, B25H, desB30 human insulin; A13N, A14E, B1E, B25H, desB30 human insulin; A(−2)G, A(−1)P, A(O)P, A14E, B25H, desB30 human insulin; A14E, B(−2)G, B(−1)P, B(O)P, B25H, desB30 human insulin; A(−2)G, A(−1)P, A(O)P, A14E, B(−2)G, B(−1)P, B(O)P, B25H, desB30 human insulin; A14E, B27R, B28D, B29K, desB30 human insulin; A14E, B25H, B27R, B28D, B29K, desB30 human insulin; A14E, B25H, B26T, B27R, B28D, B29K, desB30 human insulin; A14E, B25H, B27R, desB30 human insulin; A14E, B25H, B27H, desB30 human insulin; A14E, A18Q, B3Q, B25H, desB30 human insulin; A13E, A14E, B25H, desB30 human insulin; A12E, A14E, B25H, desB30 human insulin; A15E, A14E, B25H, desB30 human insulin; A13E, B25H, desB30 human insulin; A12E, B25H, desB30 human insulin; A15E, B25H, desB30 human insulin; A14E, B25H, desB27, desB30 human insulin; A14E, desB27, desB30 human insulin; A14H, desB27, desB30 human insulin; A14E, B16H, desB27, desB30 human insulin; A14H, B16H, desB27, desB30 human insulin; A14E, B25H, B26D, B27E, desB30 human insulin; A14E, B25H, B27R, desB30 human insulin; A14E, B25H, B27N, desB30 human insulin; A14E, B25H, B27D, desB30 human insulin; A14E, B25H, B27Q, desB30 human insulin; A14E, B25H, B27E, desB30 human insulin; A14E, B25H, B27G, desB30 human insulin; A14E, B25H, B27H, desB30 human insulin; A14E, B25H, B27K, desB30 human insulin; A14E, B25H, B27P, desB30 human insulin; A14E, B25H, B27S, desB30 human insulin; A14E, B25H, B27T, desB30 human insulin; A13R, A14E, B25H, desB30 human insulin; A13N, A14E, B25H, desB30 human insulin; A13D, A14E, B25H, desB30 human insulin; A13Q, A14E, B25H, desB30 human insulin; A13E, A14E, B25H, desB30 human insulin; A13G, A14E, B25H, desB30 human insulin; A13H, A14E, B25H, desB30 human insulin; A13K, A14E, B25H, desB30 human insulin; A13P, A14E, B25H, desB30 human insulin; A13S, A14E, B25H, desB30 human insulin; A13T, A14E, B25H, desB30 human insulin; A14E, B16R, B25H, desB30 human insulin; A14E, B16D, B25H, desB30 human insulin; A14E, B16Q, B25H, desB30 human insulin; A14E, B16E, B25H, desB30 human insulin; A14E, B16H, B25H, desB30 human insulin; A14R, B25H, desB30 human insulin; A14N, B25H, desB30 human insulin; A14D, B25H, desB30 human insulin; A14Q, B25H, desB30 human insulin; A14E, B25H, desB30 human insulin; A14G, B25H, desB30 human insulin; A14H, B25H, desB30 human insulin; A8H, B10D, B25H human insulin; and A8H, A14E, B10E, B25H, desB30 human insulin and this embodiment may, optionally, comprise B25H, desB30 human insulin and B25N, desB30 human insulin.

In a preferred embodiment, a N-terminally modified insulin according to the invention has a peptide part which is selected from the group consisting of: A14E, B25H, desB30 human insulin; A14E, B16H, B25H, desB30 human insulin; A14E, B16E, B25H, desB30 human insulin; A14E, desB27, desB30 human insulin; A14E, B16H, desB27, desB30 human insulin; A14E, B25H, B26G, B27G, B28G, desB30 human insulin; B25H, desB30 human insulin and A14E, B25H, desB27, desB30 human insulin.

In a preferred embodiment, a N-terminally modified insulin according to the invention has a peptide part which is selected from any one of the insulins mentioned above that, in addition, are containing the desB27 mutation.

In a preferred embodiment, a N-terminally modified insulin according to the invention has a peptide part which is selected from the group consisting of: A14E, B25H, desB27, desB30 human insulin; A14E, B16H, B25H, desB27, desB30 human insulin; A14E, desB27, desB30 human insulin; A14E, B16E, B25H, desB27, desB30 human insulin; and B25H, desB27, desB30 human insulin.

In one embodiment, a N-terminally modified insulin according to the invention has a peptide part which is selected from any of the above mentioned insulins and, in addition, comprise one or two of the following mutations in position A21 and/or B3 to improve chemical stability: A21G, desA21, B3Q, or B3G.

In a preferred embodiment, a N-terminally modified insulin according to the invention has a peptide part which is selected from the group consisting of: A14E, A21G, B25H, desB30 human insulin; A14E, A21G, B16H, B25H, desB30 human insulin; A14E, A21G, B16E, B25H, desB30 human insulin; A14E, A21G, B25H, desB27, desB30 human insulin; A14E, A21G, B25H, desB27, desB30 human insulin; A14E, A21G, B25H, B26G, B27G, B28G, desB30 human insulin; A21G, B25H, desB30 human insulin and A21G, B25N, desB30 human insulin, and, preferably, it is selected from the following protease stabilised insulins: A14E, A21G, B25H, desB30 human insulin; A14E, A21G, desB27, desB30 human insulin; A14E, A21G, B16H, B25H, desB30 human insulin; A14E, A21G, B16E, B25H, desB30 human insulin; A14E, A21G, B25H, desB27, desB30 human insulin; A14E, A21G, B25H, desB27, desB30 human insulin; A21G, B25H, desB30 human insulin and A21G, B25N, desB30 human insulin.

Herein, the term “acylated insulin” covers modification of insulin by attachment of one or more lipophilic substituents optionally via a linker to the insulin peptide.

A “lipophilic substituent” is herein understood as a side chain consisting of a fatty acid or a fatty diacid attached to the insulin, optionally via a linker, in an amino acid position such as LysB29, or equivalent.

In one embodiment, the “lipophilic substituent” attached to the N-terminally modified insulin has the general formula:

Acy-AA1ₙ-AA2ₘ-AA3ₚ- (Formula III),

wherein n is 0 or an integer in the range from 1 to 3; m is 0 or an integer in the range from 1 to 10; p is 0 or an integer in the range from 1 to 10; Acy is a fatty acid or a fatty diacid comprising from about 8 to about 24 carbon atoms; AA1 is a neutral linear or cyclic amino acid residue; AA2 is an acidic amino acid residue; AA3 is a neutral, alkyleneglycol-containing amino acid residue; the order by which AA1, AA2 and AA3 appears in the formula can be interchanged independently; AA2 can occur several times along the formula (e.g., Acy-AA2-AA3₂-AA2-); AA2 can occur independently (=being different) several times along the formula (e.g., Acy-AA2-AA3₂-AA2-); the connections between Acy, AA1, AA2 and/or AA3 are amide (peptide) bonds which, formally, can be obtained by removal of a hydrogen atom or a hydroxyl group (water) from each of Acy, AA1, AA2 and AA3; and attachment to the peptide part can be from the C-terminal end of a AA1, AA2, or AA3 residue in the acyl moiety of the formula (III) or from one of the side chain(s) of an AA2 residue present in the moiety of formula (III).

A non-limiting example of lipophilic substituents which may be used according to the invention may e.g. be found in the patent application WO 2009/115469, including as the lipophilic substituents of the acylated polypeptides as described in the passage beginning on page 25, line 3 of WO 2009/115469.

In one aspect of the invention, a lipophilic substituent is selected from the group consisting of:

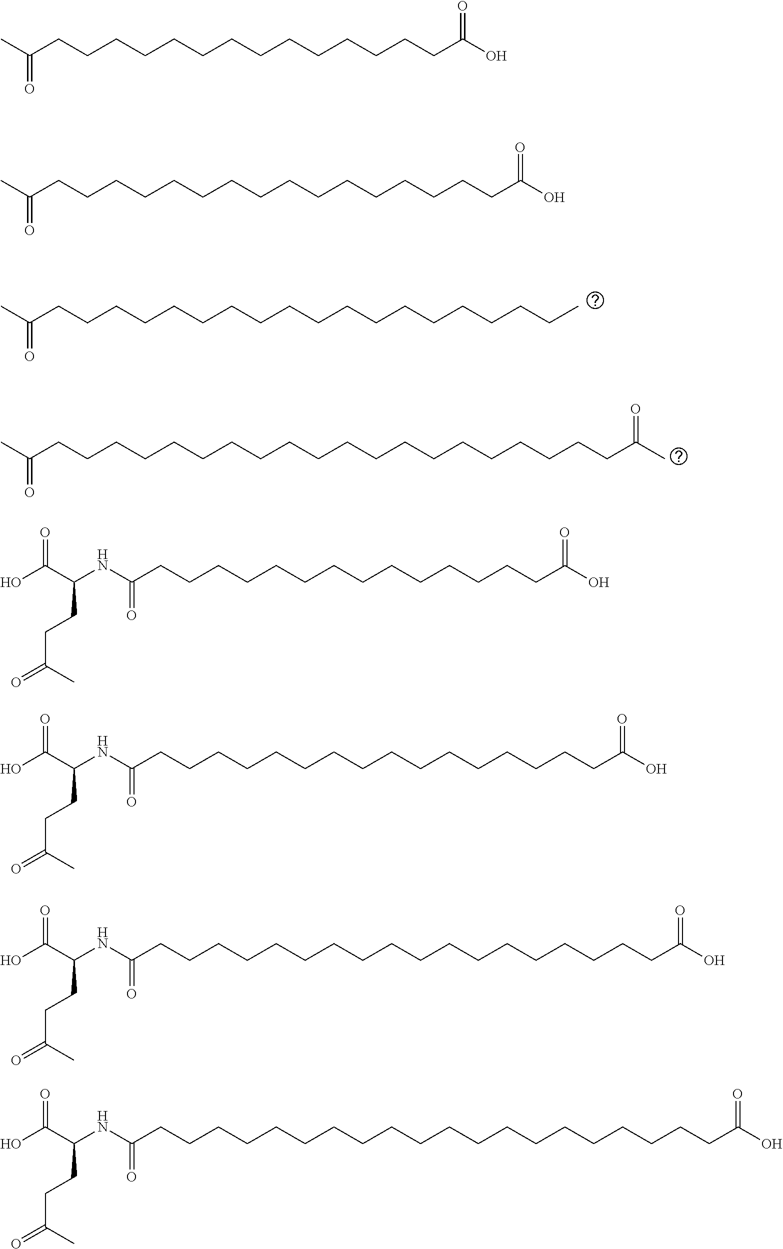

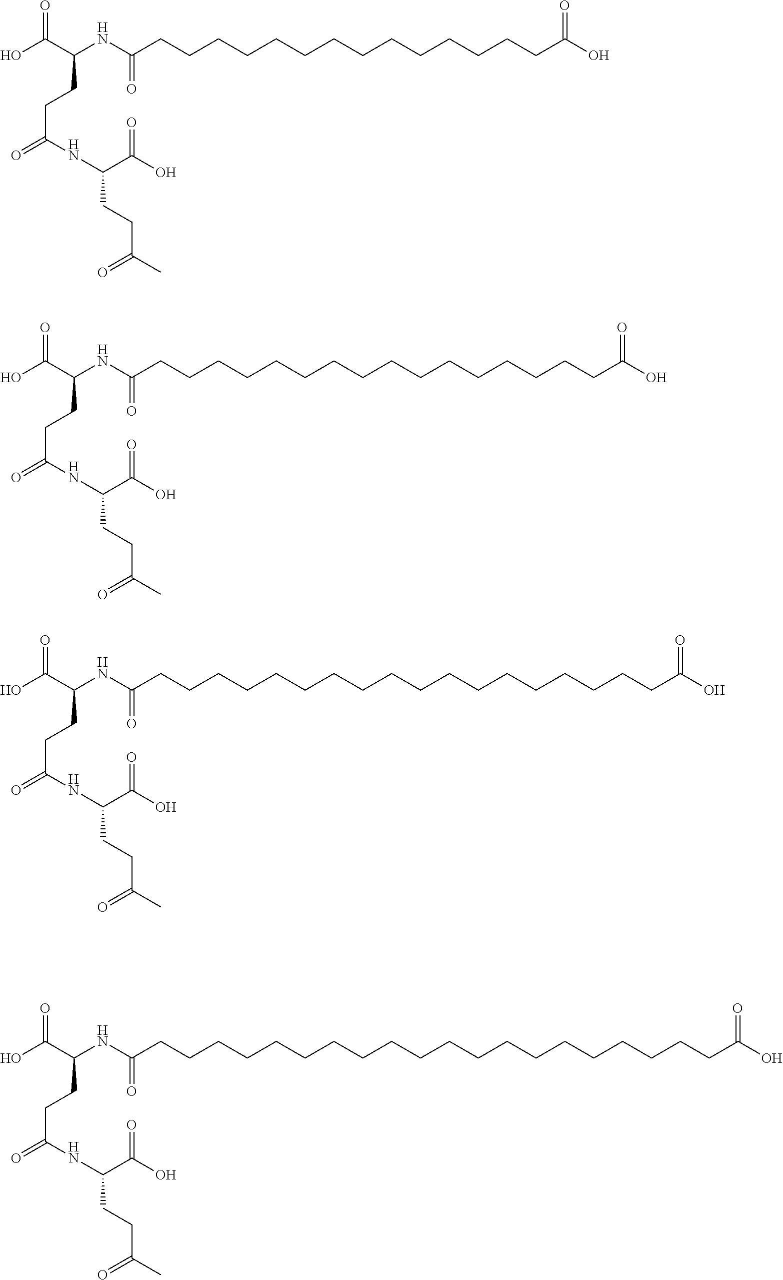 [SMILES: CC(=O)CC[C@H](NC(=O)CC[C@H](NC(=O)CCCCCCCCCCCCCCC(=O)O)C(=O)O)C(=O)O ; CC(=O)CC[C@H](NC(=O)CC[C@H](NC(=O)CCCCCCCCCCCCCCCCC(=O)O)C(=O)O)C(=O)O ; CC(=O)CC[C@H](NC(=O)CC[C@H](NC(=O)CCCCCCCCCCCCCCCCCCC(=O)O)C(=O)O)C(=O)O ; CC(=O)CC[C@H](NC(=O)CC[C@H](NC(=O)CCCCCCCCCCCCCCCCCCCCC(=O)O)C(=O)O)C(=O)O]

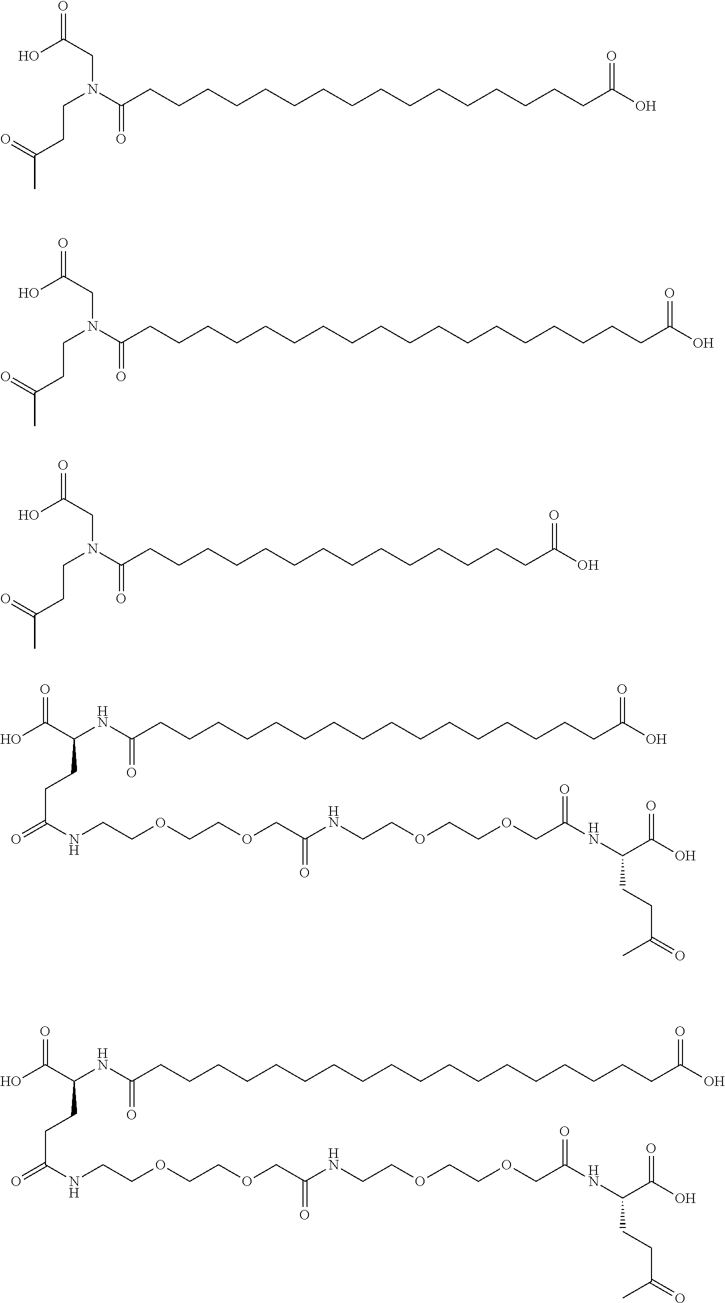

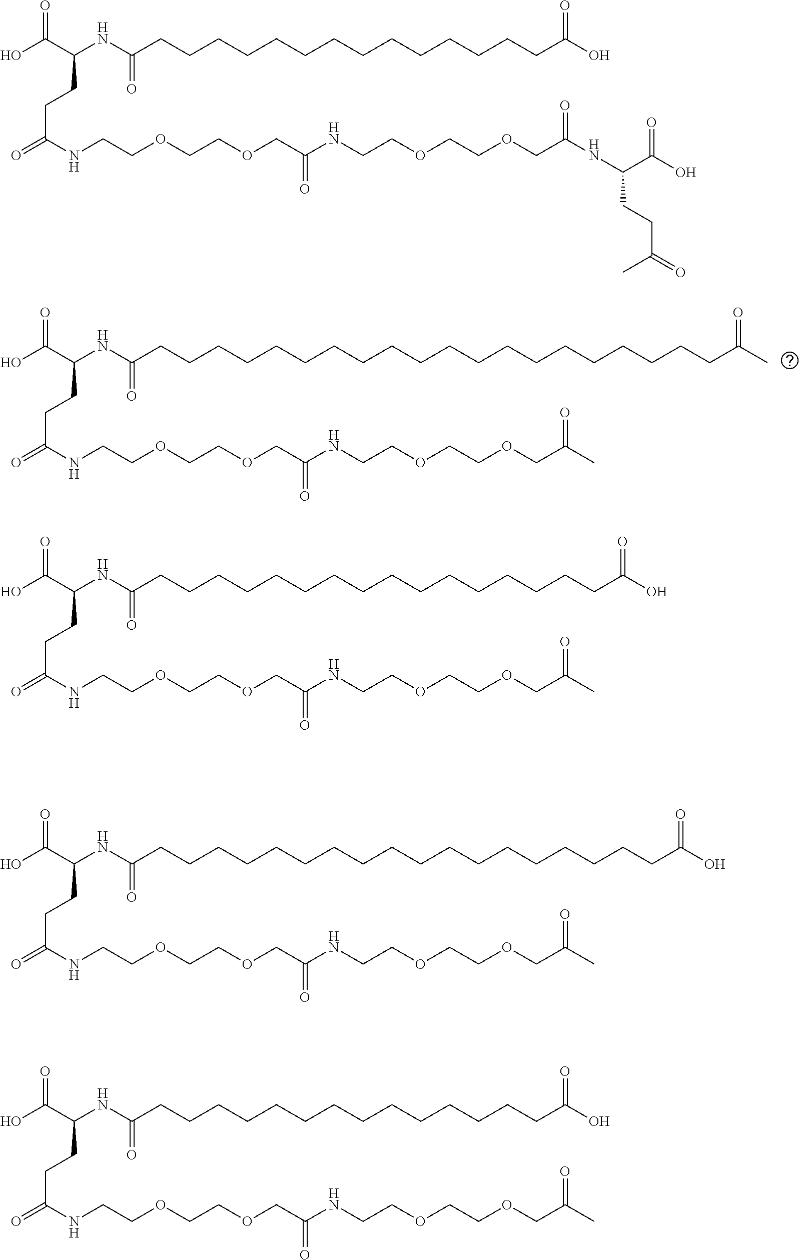 [SMILES: CC(=O)CC[C@H](NC(=O)COCCOCCNC(=O)COCCOCCNC(=O)CC[C@H](NC(=O)CCCCCCCCCCCCCCC(=O)O)C(=O)O)C(=O)O ; CC(=O)CCCCCCCCCCCCCCCCCCCCC(=O)N[C@@H](CCC(=O)NCCOCCOCC(=O)NCCOCCOCC(C)=O)C(=O)O ; CC(=O)COCCOCCNC(=O)COCCOCCNC(=O)CC[C@H](NC(=O)CCCCCCCCCCCCCCCCC(=O)O)C(=O)O ; CC(=O)COCCOCCNC(=O)COCCOCCNC(=O)CC[C@H](NC(=O)CCCCCCCCCCCCCCCCCCC(=O)O)C(=O)O ; CC(=O)COCCOCCNC(=O)COCCOCCNC(=O)CC[C@H](NC(=O)CCCCCCCCCCCCCCC(=O)O)C(=O)O]

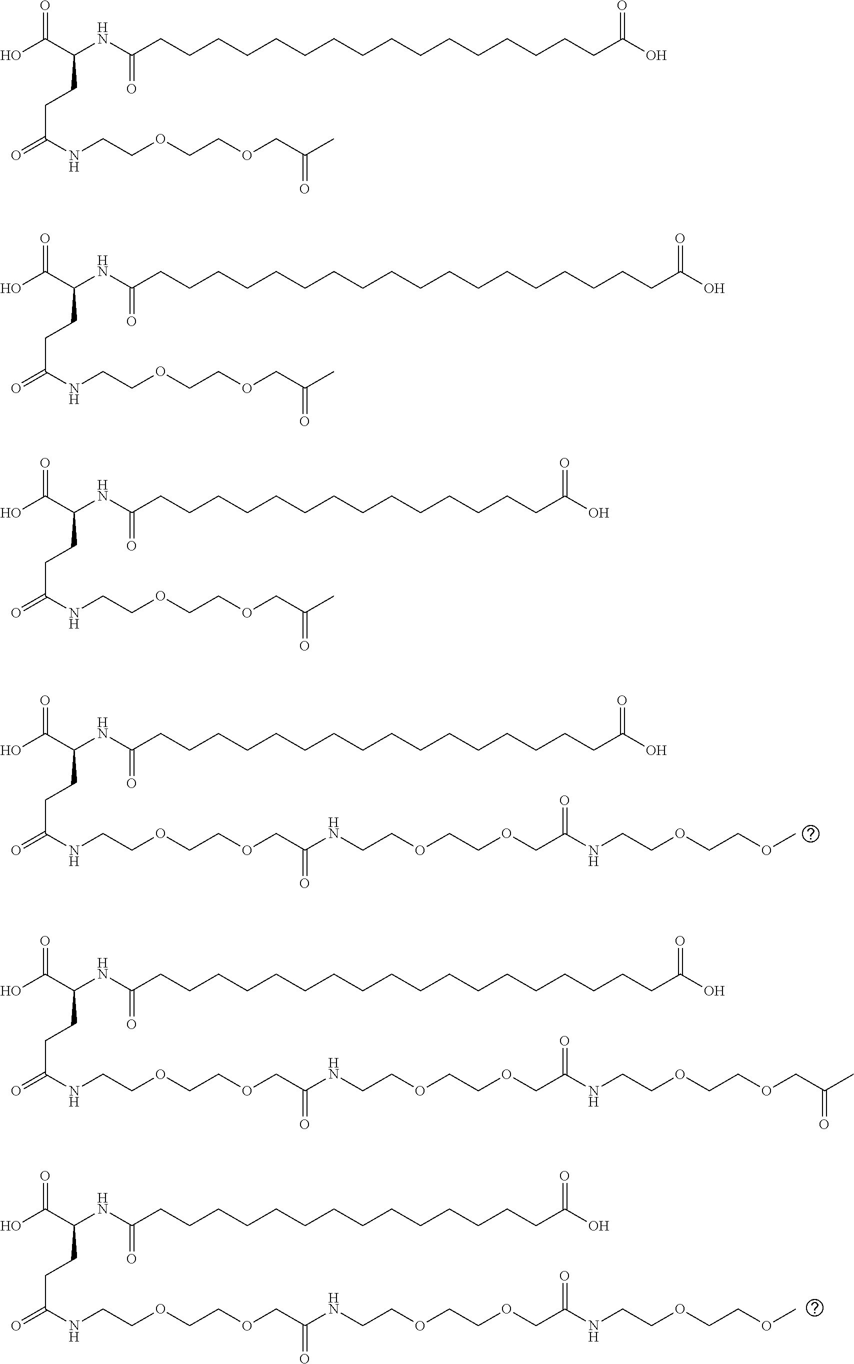 [SMILES: CC(=O)COCCOCCNC(=O)CC[C@H](NC(=O)CCCCCCCCCCCCCCCCC(=O)O)C(=O)O ; CC(=O)COCCOCCNC(=O)CC[C@H](NC(=O)CCCCCCCCCCCCCCCCCCC(=O)O)C(=O)O ; CC(=O)COCCOCCNC(=O)CC[C@H](NC(=O)CCCCCCCCCCCCCCC(=O)O)C(=O)O ; COCCOCCNC(=O)COCCOCCNC(=O)COCCOCCNC(=O)CC[C@H](NC(=O)CCCCCCCCCCCCCCCCC(=O)O)C(=O)O ; CC(=O)COCCOCCNC(=O)COCCOCCNC(=O)COCCOCCNC(=O)CC[C@H](NC(=O)CCCCCCCCCCCCCCCCCCC(=O)O)C(=O)O ; COCCOCCNC(=O)COCCOCCNC(=O)COCCOCCNC(=O)CC[C@H](NC(=O)CCCCCCCCCCCCCCC(=O)O)C(=O)O]

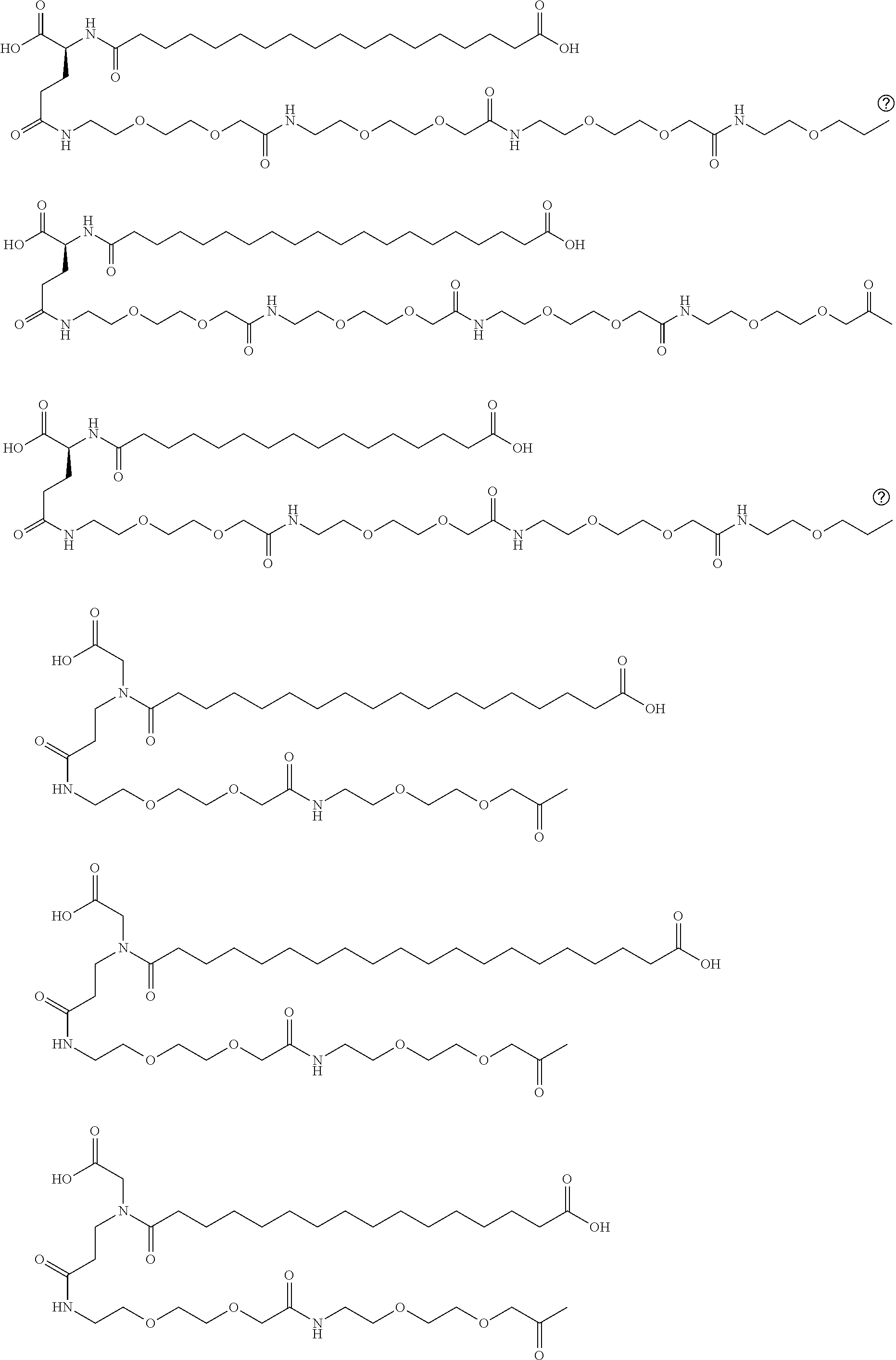

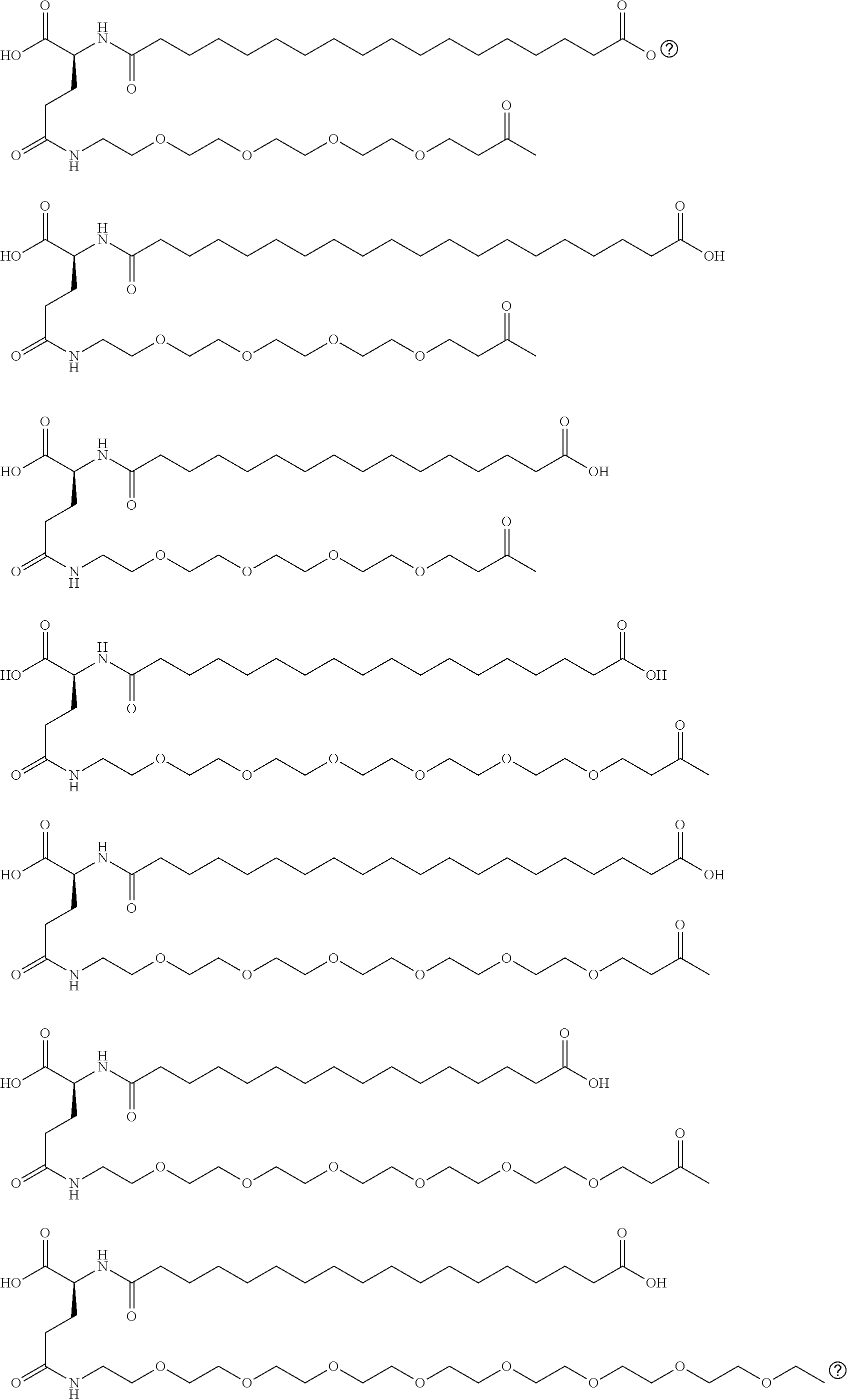

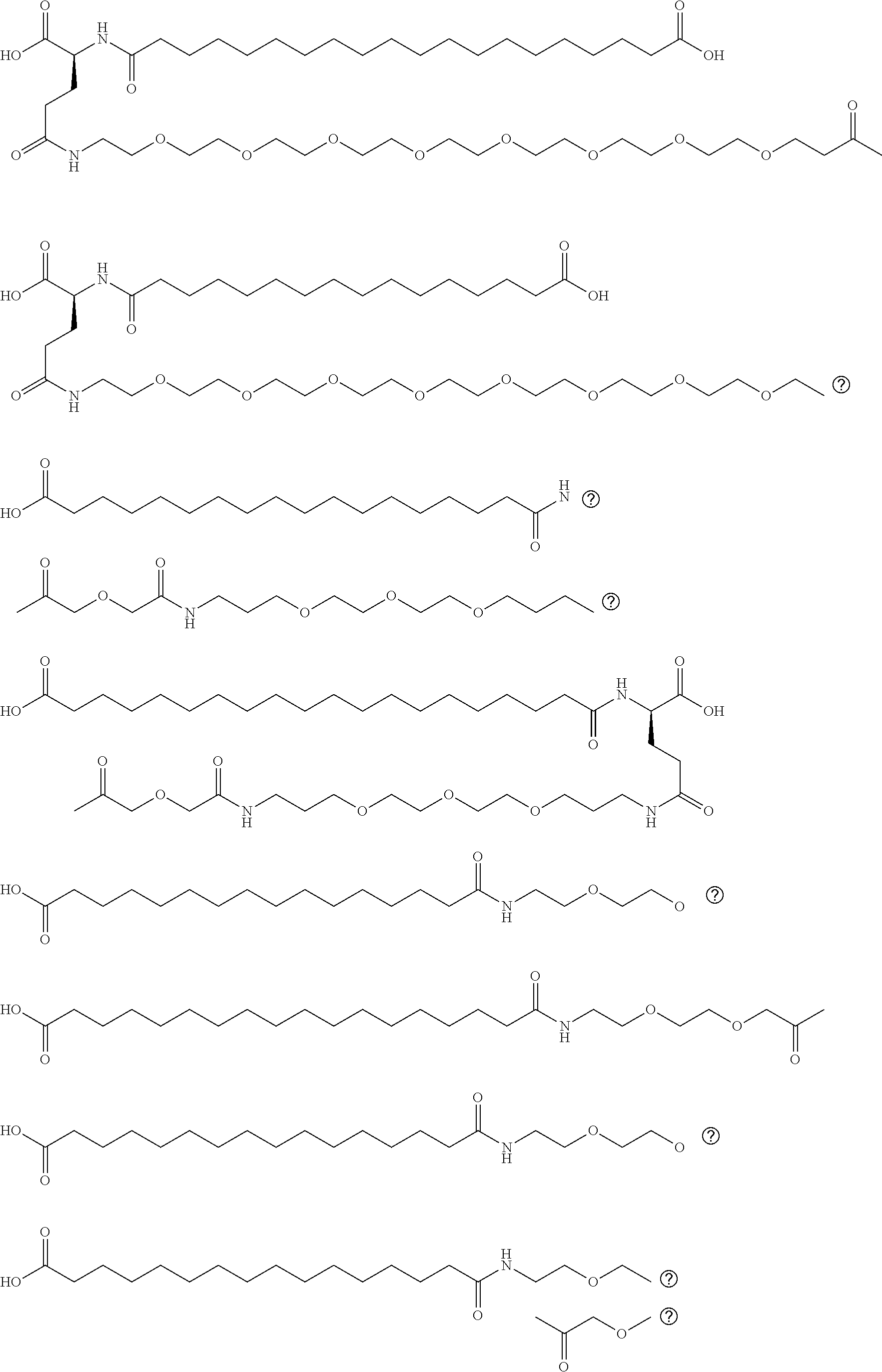

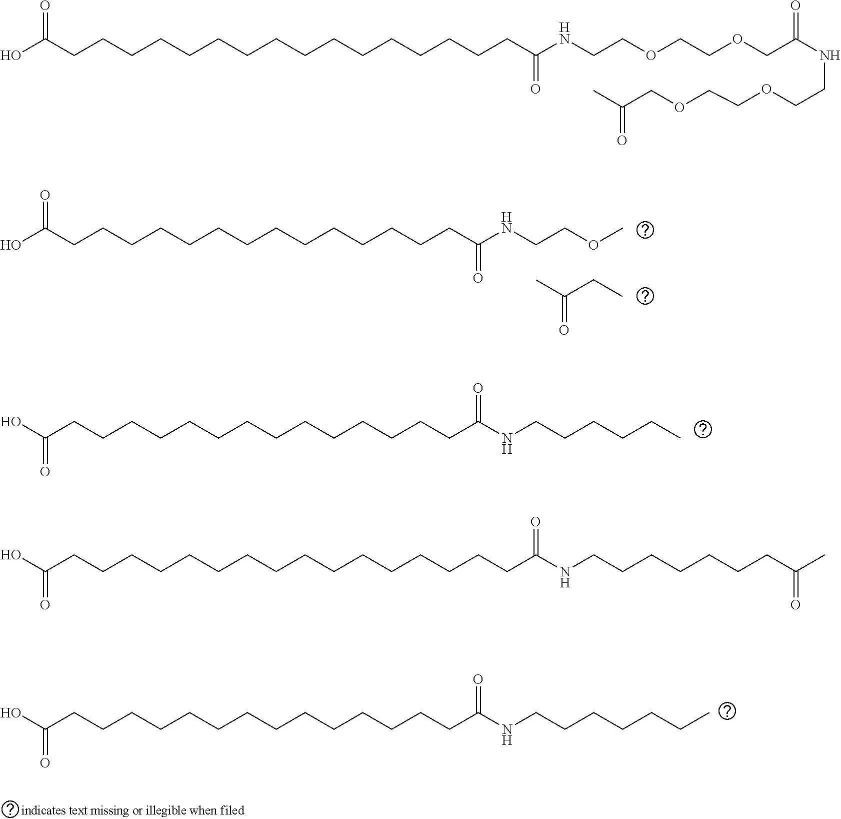

In one aspect of the invention, a lipophilic substituent is selected from the group consisting of:

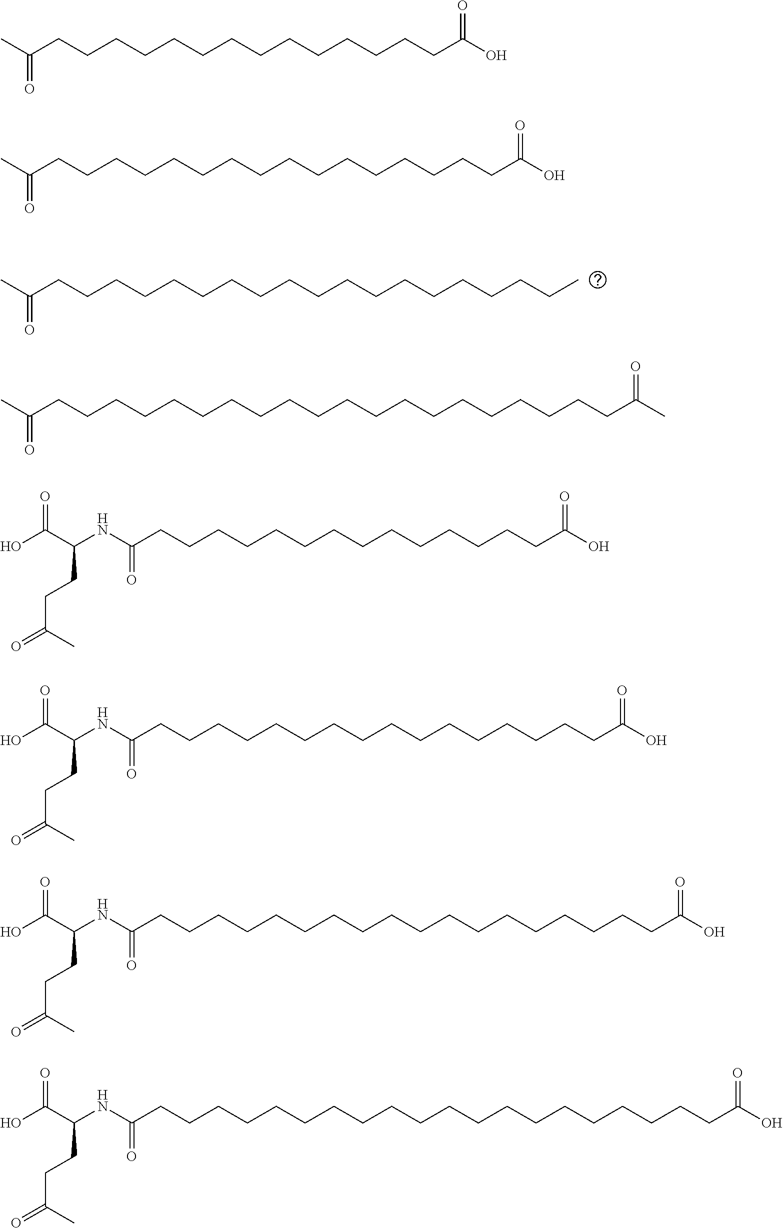

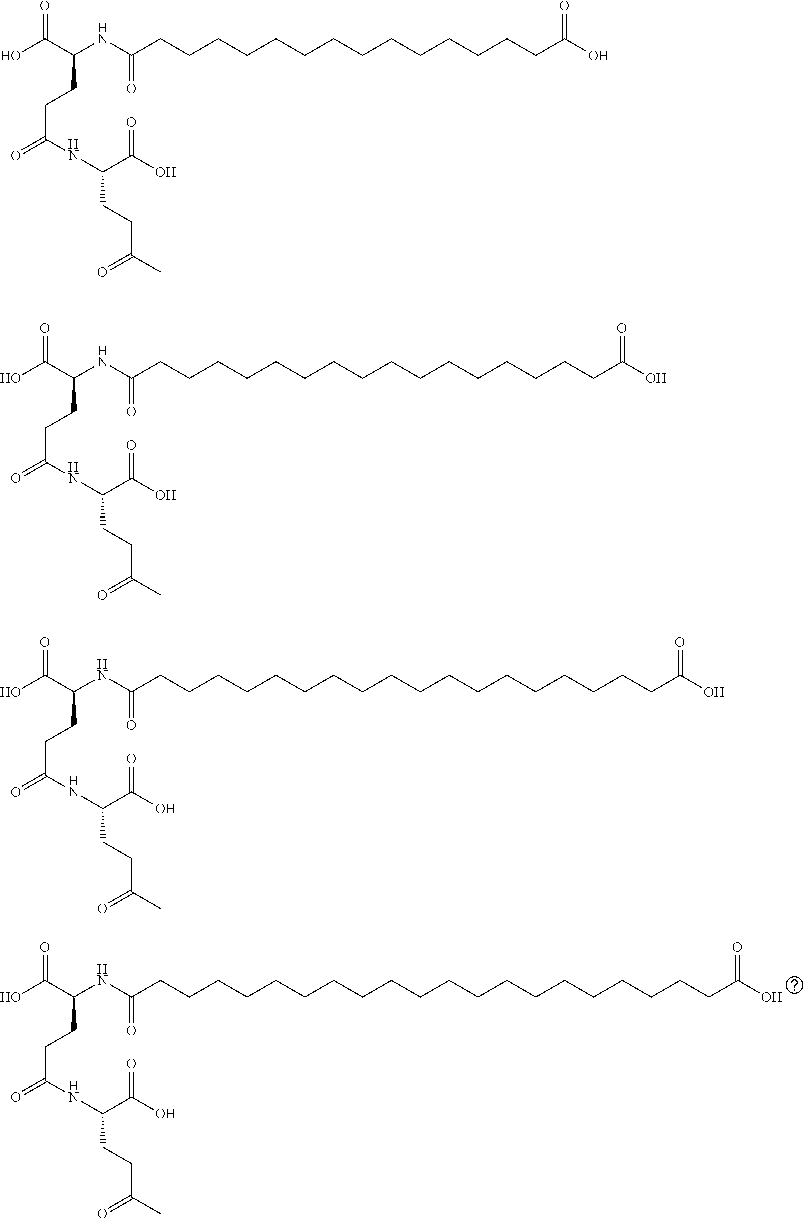 [SMILES: CC(=O)CC[C@H](NC(=O)CC[C@H](NC(=O)CCCCCCCCCCCCCCC(=O)O)C(=O)O)C(=O)O ; CC(=O)CC[C@H](NC(=O)CC[C@H](NC(=O)CCCCCCCCCCCCCCCCC(=O)O)C(=O)O)C(=O)O ; CC(=O)CC[C@H](NC(=O)CC[C@H](NC(=O)CCCCCCCCCCCCCCCCCCC(=O)O)C(=O)O)C(=O)O ; CC(=O)CC[C@H](NC(=O)CC[C@H](NC(=O)CCCCCCCCCCCCCCCCCCCCC(=O)O)C(=O)O)C(=O)O]

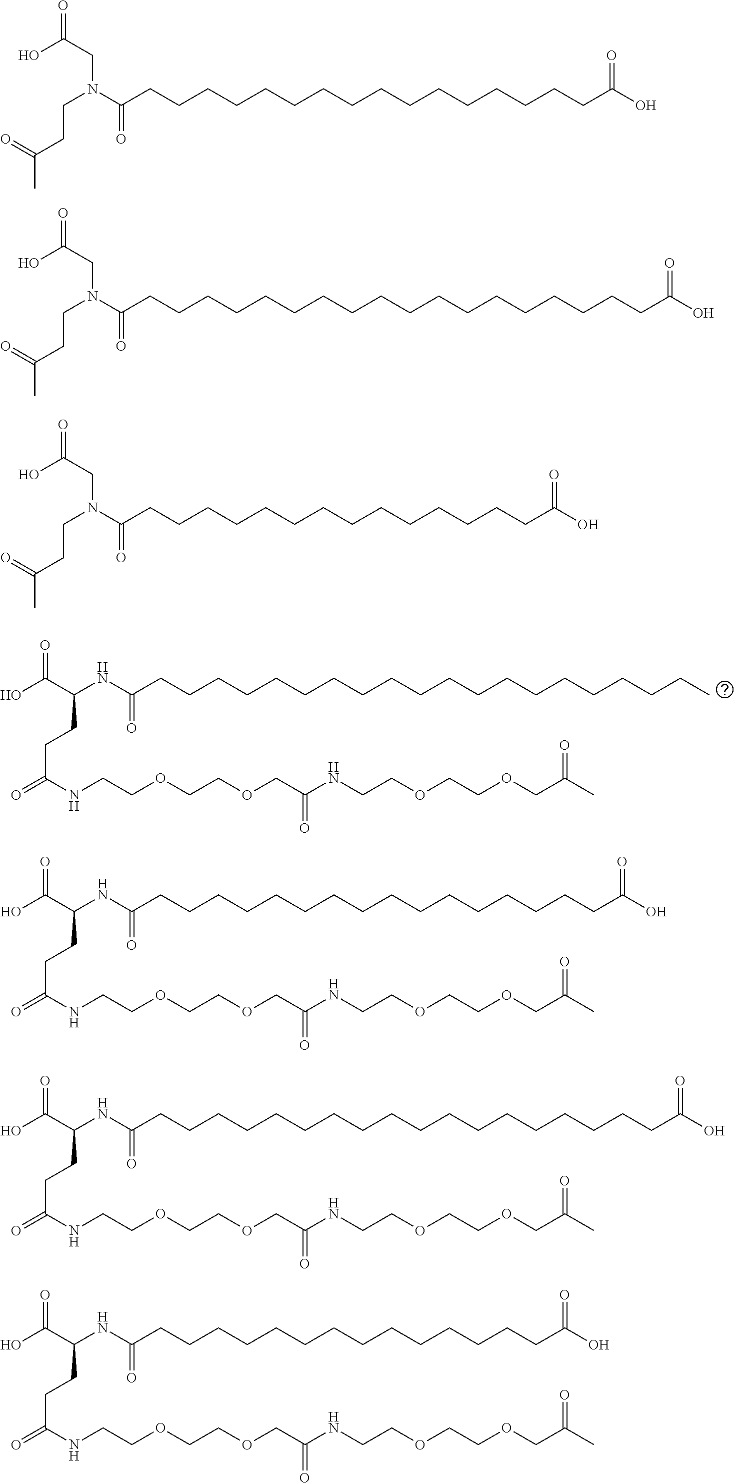

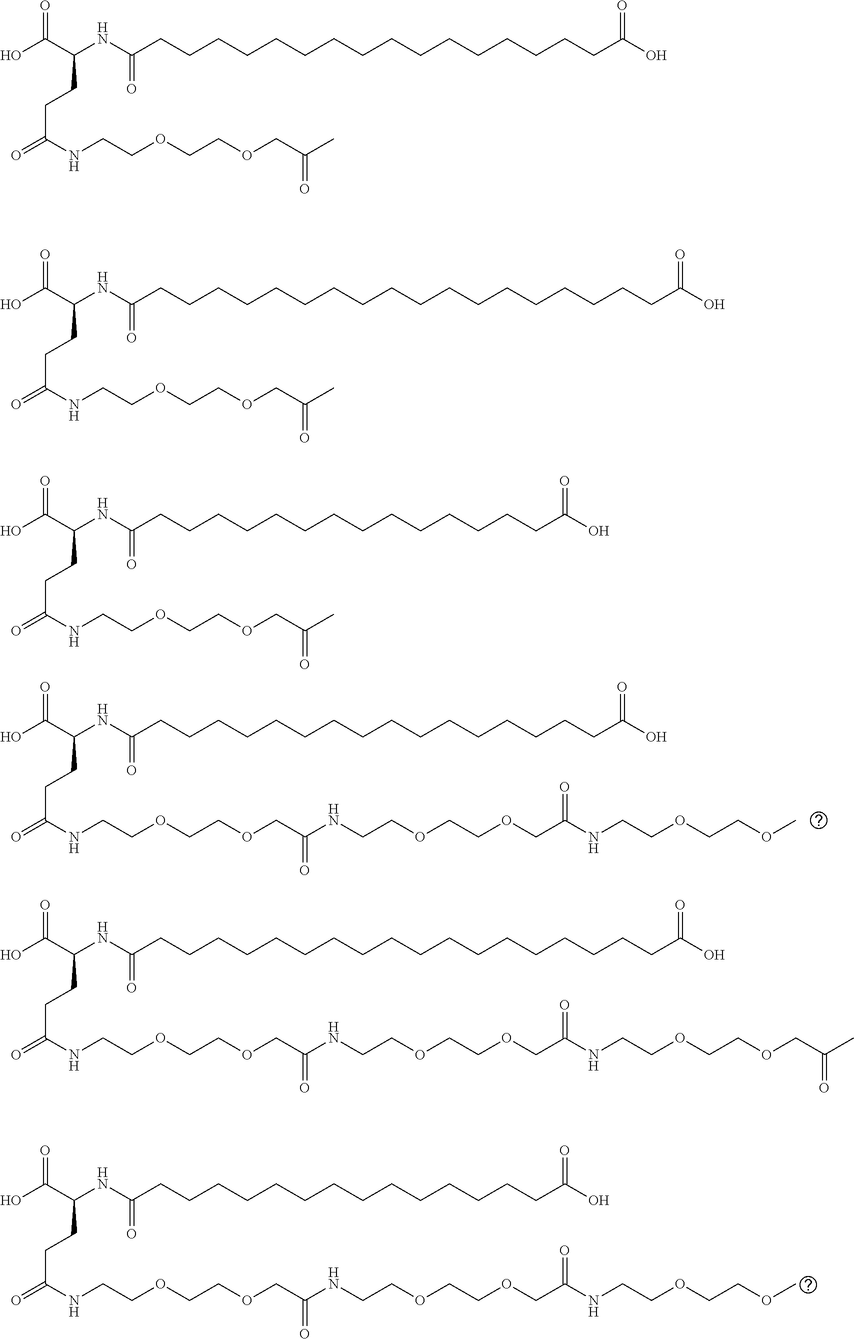 [SMILES: CC(=O)COCCOCCNC(=O)CC[C@H](NC(=O)CCCCCCCCCCCCCCCCC(=O)O)C(=O)O ; CC(=O)COCCOCCNC(=O)CC[C@H](NC(=O)CCCCCCCCCCCCCCCCCCC(=O)O)C(=O)O ; CC(=O)COCCOCCNC(=O)CC[C@H](NC(=O)CCCCCCCCCCCCCCC(=O)O)C(=O)O ; COCCOCCNC(=O)COCCOCCNC(=O)COCCOCCNC(=O)CC[C@H](NC(=O)CCCCCCCCCCCCCCCCC(=O)O)C(=O)O ; CC(=O)COCCOCCNC(=O)COCCOCCNC(=O)COCCOCCNC(=O)CC[C@H](NC(=O)CCCCCCCCCCCCCCCCCCC(=O)O)C(=O)O ; COCCOCCNC(=O)COCCOCCNC(=O)COCCOCCNC(=O)CC[C@H](NC(=O)CCCCCCCCCCCCCCC(=O)O)C(=O)O]

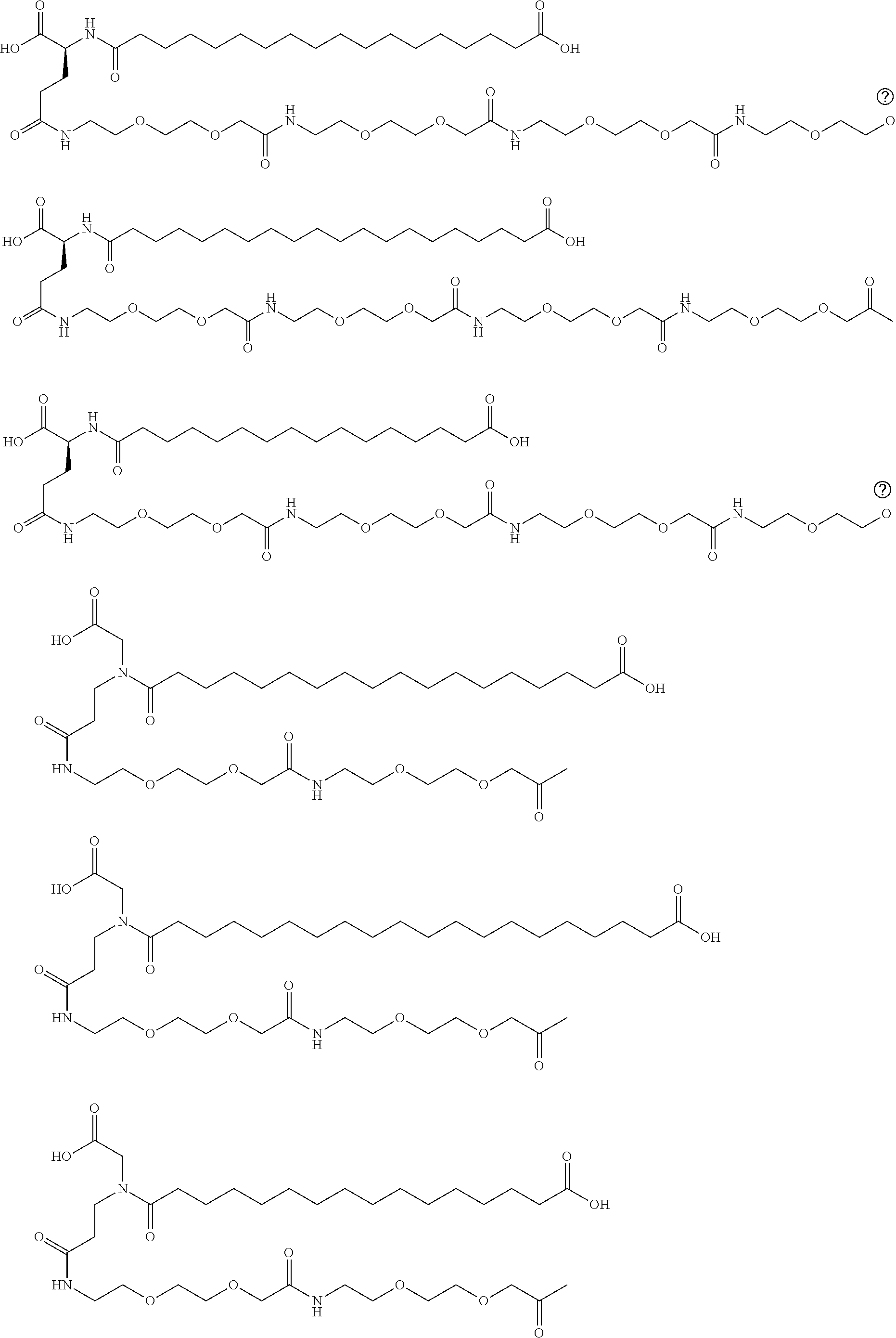

In one aspect of the invention, a lipophilic substituent is selected from the group consisting of:

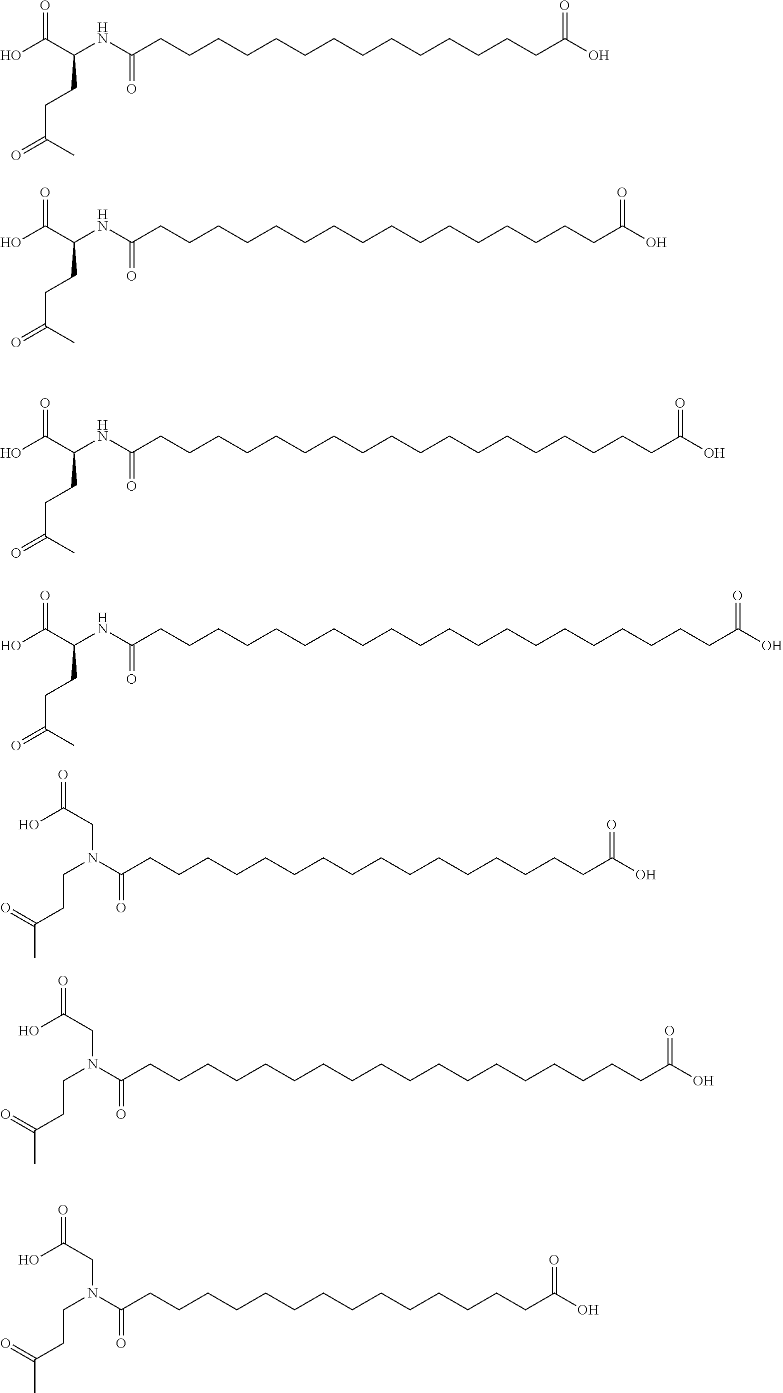

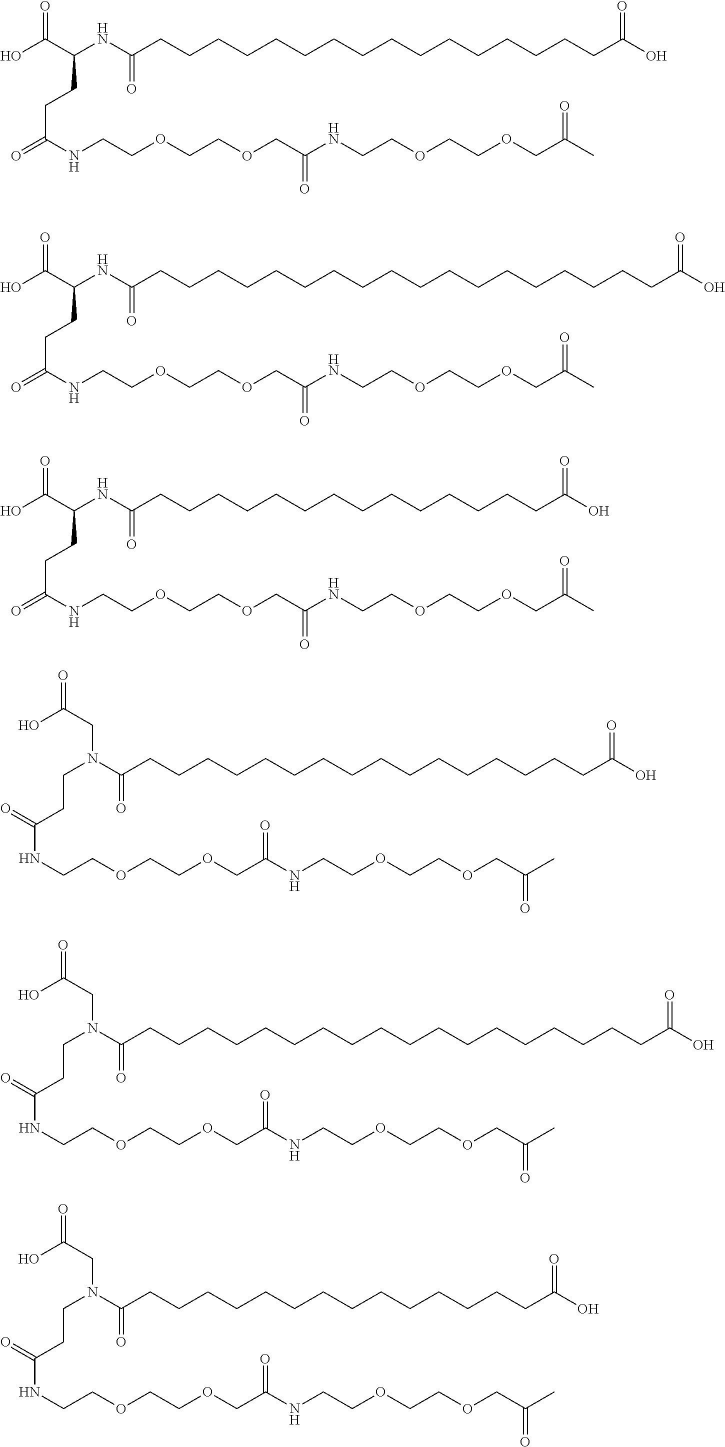 [SMILES: CC(=O)COCCOCCNC(=O)COCCOCCNC(=O)CC[C@H](NC(=O)CCCCCCCCCCCCCCCCC(=O)O)C(=O)O ; CC(=O)COCCOCCNC(=O)COCCOCCNC(=O)CC[C@H](NC(=O)CCCCCCCCCCCCCCCCCCC(=O)O)C(=O)O ; CC(=O)COCCOCCNC(=O)COCCOCCNC(=O)CC[C@H](NC(=O)CCCCCCCCCCCCCCC(=O)O)C(=O)O ; CC(=O)COCCOCCNC(=O)COCCOCCNC(=O)CCN(CC(=O)O)C(=O)CCCCCCCCCCCCCCCCC(=O)O ; CC(=O)COCCOCCNC(=O)COCCOCCNC(=O)CCN(CC(=O)O)C(=O)CCCCCCCCCCCCCCCCCCC(=O)O ; CC(=O)COCCOCCNC(=O)COCCOCCNC(=O)CCN(CC(=O)O)C(=O)CCCCCCCCCCCCCCC(=O)O]

An “N-terminally modified insulin” is herein the same as an “N-terminally protected insulin” and is defined as an insulin comprising one or more N-terminal modification groups also herein named N-terminal protecting groups.

“N-terminal modification groups” are herein the same as “N-terminal protecting groups” and according to the invention are groups that, when conjugated to the N-terminal amino groups of the A- and/or B-chain of the insulin, protect said amino groups of the N-terminal amino acids of the insulin (typically, but not always), glycine and phenylalanine of the A- and the B-chain, respectively, from reacting with e.g. aldehyde impurities of one or more of the excipients in a pharmaceutical formulation. In one aspect of the invention the N-terminal modification is one or two organic substituents having a MW below 200 g per mol conjugated to an N-terminal of the parent insulin”.

In one aspect the N-terminally modified insulin derivative of the invention comprises the N-terminal modification groups Y and Z attached to at least one, preferably two N-terminal amino acid(s) as illustrated in formula I with the first four residues of the insulin A-chain shown (GIVE . . . ).

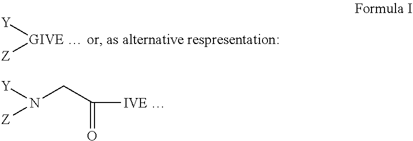

In one aspect of the invention, Y and Z are different and:
Y is R—C(═X)—,
Z is H,
R is H, NH₂, straight chain or branched C1-C4 alkyl, (optionally substituted with dimethylamino, diethylamino, dipropylamino, trimethylammonium, triethylammonium, or tripropylammonium), C5-C6 cycloalkyl (optionally substituted), 5- or 6 membered saturated heterocyclyl (optionally substituted), and
X is O or S.

In one aspect of the invention, when Y is R—C(═X)— and Z is H, the insulin can contain the desA1 and desB1 mutations.

In another aspect of the invention, Y═Z is C1-C4 alkyl.

In one aspect of the invention, each of the N-terminal protecting groups of the A- and the B-chain N-terminal amino groups are the same.

In one aspect of the invention, each of the two N-terminal protecting groups of the invention is having a molecular weight below 150 Da.

In one aspect of the invention, each of the N-terminal protecting groups of the invention is positively charged at physiological pH, i.e. when the N-terminal modification group is attached/conjugated to the N-terminal amino group, the amino group, or the substituent on the amino group, has a positive charge. In one aspect of the invention, the N-terminal protecting groups are selected from the group consisting of: Dimethyl, diethyl, di-n-propyl, disec-propyl, di-n-butyl, di-1-butyl or the like. In another aspect of the invention, the N-terminal protecting groups are selected from dimethyl and diethyl. In another aspect of the invention, the N-terminal protecting group is dimethyl.

In one aspect of the invention, the N-terminal protecting groups are selected from the group consisting of: N,N-Dimethylglycyl, N,N-dimethylaminobutanoyl, N,N-dimethylaminopropionyl and 3-(1-piperidinyl)propionyl.

In one aspect of the invention, each of the N-terminal protecting groups of the invention removes the normal positive (or partly positive) charge of the N-terminal amino groups at physiological pH. In one aspect of the invention, each of the N-terminal protecting groups of the invention is selected from small acyl residues. In one aspect of the invention, each of the N-terminal protecting groups of the invention is selected from formyl, acetyl, propanoyl, and butanoyl groups. In one aspect of the invention, each of the N-terminal protecting groups of the invention is selected from cyclic acyl residues, e.g. the pyroglutaminyl (=5-oxopyrrolidine-2-oyl) group.

In one aspect of the invention, each of the N-terminal protecting groups of the invention removes the normal positive (or partly positive) charge of the N-terminal amino groups at physiological pH. In one aspect of the invention, each of the N-terminal protecting groups of the invention is selected from carbamoyl and thiocarbamoyl. In one aspect of the invention, each of the N-terminal protecting groups of the invention is carbamoyl.

In one aspect of the invention, each of the N-terminal protecting groups of the invention removes the normal positive (or partly positive) charge of the N-terminal amino groups at physiological pH. In one aspect of the invention, each of the N-terminal protecting groups of the invention is selected from oxalyl, glutaryl, or diglycolyl (other names: 3-oxoglutaryl, carboxymethoxyacetyl). In one aspect of the invention, each of the N-terminal protecting groups of the invention is selected from glutaryl and diglycolyl (other names: 3-oxoglutaryl, carboxymethoxyacetyl). In one aspect of the invention, each of the N-terminal protecting groups of the invention is glutaryl. In one aspect of the invention, each of the N-terminal protecting groups of the invention is diglycolyl (other names: 3-oxoglutaryl, carboxymethoxyacetyl).

When used herein, the term “conjugate” is intended to indicate the process of bonding a substituent to a polypeptide to modify the properties of said polypeptide. “Conjugation” or a “conjugation product” of a molecule and a polypeptide is thus a term for said substituent bonded to an amino acid of the polypeptide and a “substituent” as described herein thus means the substituent which is attached to the polypeptide.

“Monoalkylation” is herein to be understood as conjugation of one alkyl substituent to a free amino group of a polypeptide and “dialkylation” is to be understood as conjugation of two alkyl substituents to a free amino group of a polypeptide as illustrated below, where a “free amino group” is to be understood as a primary amine, R—NH2, or a secondary amine, R1-NH—R2, where R, R1 and R2 represents a substituent.

“Guadinylation” is herein to be understood as conjugation of an amidinyl substituent (which may also be referred to as carboxamidine, i.e. a substitutent of the form: RₙC(═NR)NR₂, where Rₙ is the polypeptide) to a free amino group of the polypeptide resulting in transformation of the amino group to a guadinyl group as illustrated below.

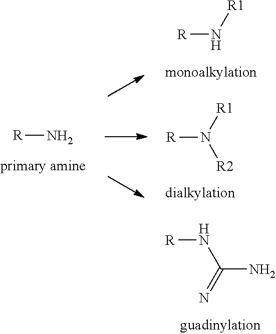

With “insulin”, “an insulin” or “the insulin” as used herein is meant human insulin, porcine insulin or bovine insulin with disulfide bridges between CysA7 and CysB7 and between CysA20 and CysB19 and an internal disulfide bridge between CysA6 and CysA11 or an insulin analogue or derivative thereof.

Human insulin consists of two polypeptide chains, the A and B chains which contain 21 and 30 amino acid residues, respectively. The A and B chains are interconnected by two disulphide bridges. Insulin from most other species is similar, but may contain amino acid substitutions in some positions.

An insulin analogue as used herein is a polypeptide which has a molecular structure which formally can be derived from the structure of a naturally occurring insulin, for example that of human insulin, by deleting and/or substituting at least one amino acid residue occurring in the natural insulin and/or by adding at least one amino acid residue.

In one aspect an insulin analogue according to the invention comprises less than 8 modifications (substitutions, deletions, additions) relative to human insulin. In one aspect an insulin analogue comprises less than 7 modifications (substitutions, deletions, additions) relative to human insulin. In one aspect an insulin analogue comprises less than 6 modifications (substitutions, deletions, additions) relative to human insulin. In another aspect an insulin analogue comprises less than 5 modifications (substitutions, deletions, additions) relative to human insulin. In another aspect an insulin analogue comprises less than 4 modifications (substitutions, deletions, additions) relative to human insulin. In another aspect an insulin analogue comprises less than 3 modifications (substitutions, deletions, additions) relative to human insulin. In another aspect an insulin analogue comprises less than 2 modifications (substitutions, deletions, additions) relative to human insulin.

A derivative of insulin is a naturally occurring human insulin or an insulin analogue which has been chemically modified, e.g. by introducing a side chain in one or more positions of the insulin backbone or by oxidizing or reducing groups of the amino acid residues in the insulin or by converting a free carboxylic group to an ester group or to an amide group. Other derivatives are obtained by acylating a free amino group or a hydroxy group, such as in the B29 position of human insulin or desB30 human insulin.

A derivative of insulin is thus human insulin or an insulin analogue which comprises at least one covalent modification such as a side-chain attached to one or more amino acids of the insulin peptide.

Herein, the naming of the insulins is done according to the following principles: The names are given as mutations and modifications (acylations) relative to human insulin. For the naming of the acyl moiety, the naming is done as peptide nomenclature. For example, naming the acyl moiety:

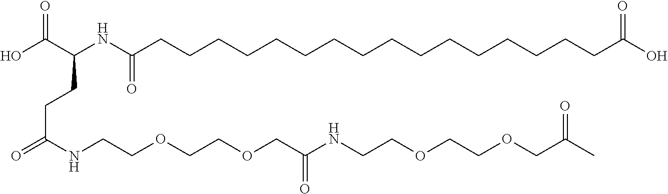 [SMILES: CC(=O)COCCOCCNC(=O)COCCOCCNC(=O)CC[C@H](NC(=O)CCCCCCCCCCCCCCCCC(=O)O)C(=O)O]

can be e.g. “octadecanedioyl-γ-L-Glu-OEG-OEG”, “octadecanedioyl-γGlu-2xOEG”, “octadecanedioyl-gGlu-2xOEG”, “17-carboxyheptadecanoyl-γ-L-Glu-OEG-OEG”, or “17-carboxyheptadecanoyl-γ-L-Glu-2xOEG”, wherein

OEG is short hand notation for the amino acid residue—NH(CH₂)₂O(CH₂)₂OCH₂CO—,

α-L-Glu (alternatively notated g-L-Glu, gGlu, γGlu or gamma-L-Glu) is short hand notation for the L-form of the amino acid gamma glutamic acid moiety.

If the enantiomer form of the gamma glutamic acid moiety is not specified, the moiety may be in the form of a pure enantiomer wherein the stereo configuration of the chiral amino acid moiety is either D or L (or if using the R/S terminology: either R or S) or it may be in the form of a mixture of enantiomers (D and L/R and S).

The acyl moiety of the modified peptides or proteins may be in the form of a pure enantiomer wherein the stereo configuration of the chiral amino acid moiety is either D or L (or if using the R/S terminology: either R or S) or it may be in the form of a mixture of enantiomers (D and L/R and S). In one aspect of the invention the acyl moiety is in the form of a mixture of enantiomers. In one aspect the acyl moiety is in the form of a pure enantiomer. In one aspect the chiral amino acid moiety of the acyl moiety is in the L form. In one aspect the chiral amino acid moiety of the acyl moiety is in the D form.

With “desB30 human insulin” is meant an analogue of human insulin lacking the B30 amino acid residue. Similarly, “desB29desB30 human insulin” means an analogue of human insulin lacking the B29 and B30 amino acid residues. With “B1”, “A1” etc. is meant the amino acid residue at position 1 in the B-chain of insulin (counted from the N-terminal end) and the amino acid residue at position 1 in the A-chain of insulin (counted from the N-terminal end), respectively. The amino acid residue in a specific position may also be denoted as e.g. PheB1 which means that the amino acid residue at position B1 is a phenylalanine residue.

For example, the insulin of example 1 (with the sequence/structure given below) is named “A1(N^{α},N^{α}-Dimethyl), A14E, B1(N^{α},N^{α}-dimethyl), B25H, B29K(N^{ε}octadecanedioyl-gGlu-2xOEG), desB30 human insulin” to indicate that the amino acid in position A14, Y in human insulin, has been mutated to E, the amino acid in position B25, F in human insulin, has been mutated to H, the amino acids in position A1 and B1 (glycine and phenylalanine, respectively) have been modified by (formally) dimethylation of the N-terminal (alpha) amino groups, the amino acid in position B29, K as in human insulin, has been modified by acylation on the epsilon nitrogen in the lysine residue of B29, denoted N^{ε}, by the residue octadecanedioyl-γGlu-2xOEG, and the amino acid in position B30, T in human insulin, has been deleted. Asterisks in the formula below indicate that the residue in question is different (i.e. mutated) as compared to human insulin. Alternatively, the insulin of example 1 (with the sequence/structure given below) can also be named “A1G(N^{α},N^{α}-Dimethyl), A14E, B1F(N^{α},N^{α}dimethyl), B25H, B29K(N^{ε}octadecanedioyl-gGlu-2xOEG), desB30 human insulin” to further indicate the amino acid residues in position A1 and B1 are G (Gly) and F (Phe), respectively. Furthermore, the notations “N^{α}” and “N^{ε}” can also be written as “N(alpha)” or “N(a)”, and as “N(epsilon)” or “N(eps)”, respectively.

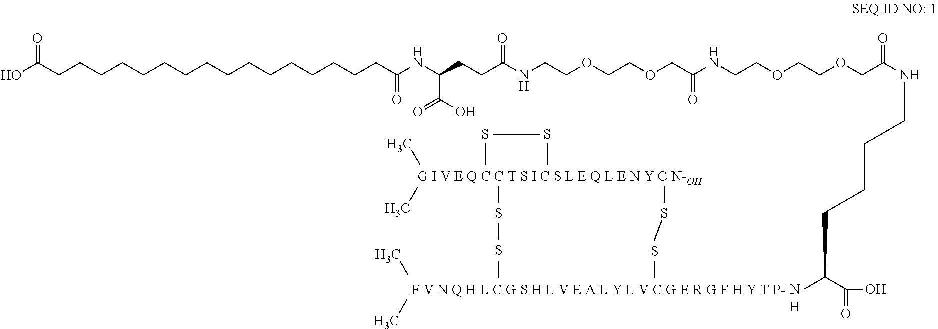

The same insulin may also be illustrated in an alternative representation:

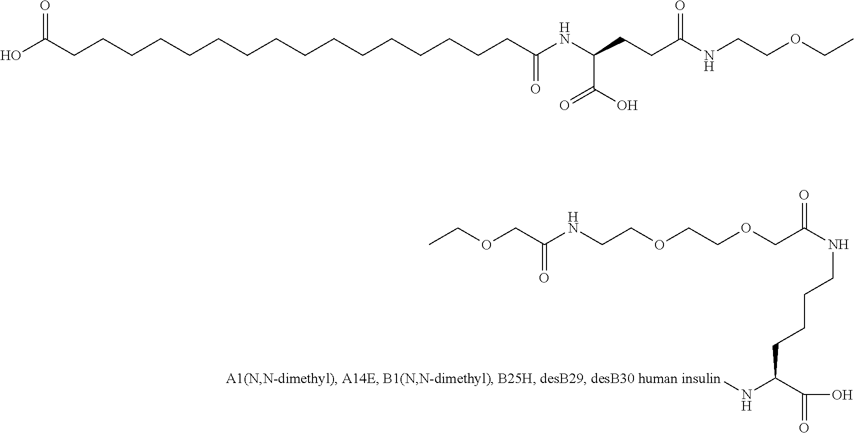

In addition, the insulins of the invention are also named according to IUPAC nomenclature (OpenEye, IUPAC style). According to this nomenclature, the above acylated N-terminally modified insulin is assigned the following name:

N{A1},N{A1}-dimethyl,N{B1},N{B1}-dimethyl,N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]-ethoxy]ethoxy]acetyl]-[GluA14,HisB25],des-ThrB30-Insulin (human)

Notation of N-terminal modifications:

The N-terminal modifications are drawn without the alpha amino group and is to be understood as indicated in the examples below.

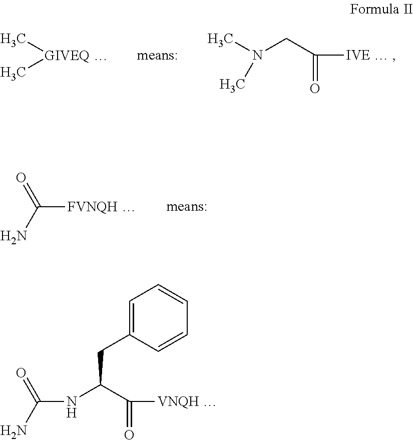

The production of polypeptides is well known in the art. Polypeptides, such as the peptide part of an N-terminal modified insulin according to the invention, may for instance be produced by classical peptide synthesis, e.g. solid phase peptide synthesis using t-Boc or Fmoc chemistry or other well established techniques, see e.g. Greene and Wuts, “Protective Groups in Organic Synthesis”, John Wiley & Sons, 1999. The polypeptides may also be produced by a method which comprises culturing a host cell containing a DNA sequence encoding the polypeptide and capable of expressing the polypeptide in a suitable nutrient medium under conditions permitting the expression of the peptide. For polypeptides comprising non-natural amino acid residues, the recombinant cell should be modified such that the non-natural amino acids are incorporated into the polypeptide, for instance by use of tRNA mutants.

The term “stability” is herein used for a pharmaceutical composition comprising a N-terminally modified insulin to describe the shelf life of the composition. The term “stabilized” or “stable” when referring to a N-terminally modified insulin thus refers to a composition with increased chemical stability or increased physical and chemical stability relative to a composition comprising an insulin which is not N-terminally modified.

The term “chemical stability” of a N-terminally modified insulin as used herein refers to chemical covalent changes in the protein structure leading to formation of chemical degradation products with potential less biological potency and/or potential increased immunogenic properties compared to the native protein structure. Various chemical degradation products can be formed depending on the type and nature of the native protein and the environment to which the protein is exposed. Elimination of chemical degradation can most probably not be completely avoided and increasing amounts of chemical degradation products is often seen during storage and use of the pharmaceutical composition as well-known by the person skilled in the art. Most proteins are prone to deamidation, a process in which the side chain amide group in glutaminyl or asparaginyl residues is hydrolysed to form a free carboxylic acid. Other degradations pathways involves formation of high molecular weight transformation products where two or more protein molecules are covalently bound to each other through transamidation and/or disulfide interactions leading to formation of covalently bound dimer, oligomer and polymer degradation products (Stability of Protein Pharmaceuticals, Ahern. T. J. & Manning M. C., Plenum Press, New York 1992). Oxidation can be mentioned as another variant of chemical degradation. The chemical stability of the N-terminally modified insulin can be evaluated by measuring the amount of the chemical degradation products at various time-points after exposure to different environmental conditions (the formation of degradation products can often be accelerated by for instance increasing temperature). The amount of each individual degradation product is often determined by separation of the degradation products depending on molecule size, hydrophilicity, hydrophobicity, and/or charge using various chromatography techniques (e.g. SEC-HPLC and/or RP-HPLC).

Hence, as outlined above, “stabilized” or “stable” when referring to a N-terminally modified insulin refers to a N-terminally modified insulin with increased chemical stability or increased physical and chemical stability. In general, a pharmaceutical composition must be stable during use and storage (in compliance with recommended use and storage conditions) until the expiration date is reached.

In one aspect of the invention a pharmaceutical composition, such as a lipid pharmaceutical composition, comprising the N-terminally modified insulin is stable for more than 6 weeks of usage and for more than 2 years of storage.

In another aspect of the invention a pharmaceutical composition, such as a lipid pharmaceutical composition, comprising the N-terminally modified insulin is stable for more than 4 weeks of usage and for more than two years of storage.

In a further aspect of the invention a pharmaceutical composition, such as a lipid pharmaceutical composition, comprising the N-terminally modified insulin is stable for more than 4 weeks of usage and for more than 3 years of storage.

In an even further aspect of the invention a pharmaceutical composition, such as a lipid pharmaceutical composition, comprising the N-terminally modified insulin is stable for more than 2 weeks of usage and for more than two years of storage.

### the Following is a Non-Limiting List of Aspects According to the Invention:

1. An N-terminally modified insulin, wherein the insulin is an acylated, protease stabilised insulin and the N-terminal modification is with one or more N-terminal modification groups that are positively charged at physiological pH.
2. An N-terminally modified insulin according to aspect 1, wherein the N-terminally modified insulin consists of a peptide part, a lipophilic substituent and an N-terminal modification group.
3. An N-terminally modified insulin according to aspect 1 or 2, wherein the positively charged modification groups at physiological pH are one or two organic substituents which are positively charged at physiological pH and are having a MW below 200 g per mol conjugated to the N-terminals of the parent insulin.
4. An N-terminally modified insulin according to any one of the previous aspects, wherein the positively charged modification groups at physiological pH are designated Y and Z in

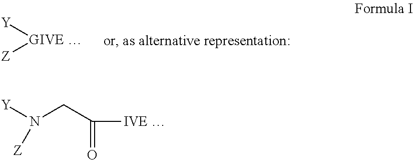

and wherein Y and Z are attached to at the N-terminal amino acids of the insulin peptide.
5. An N-terminally modified insulin according to aspect 4, wherein Y and Z are different and
Y is straight chain or branched C1-C4 alkyl, straight chain or branched C2-C4 acyl substituted with dimethylamino, diethylamino, dipropylamino, trimethylammonium, triethylammonium or dipropylammonium, 5- or 6 membered saturated heterocyclyl, substituted 5- or 6 membered saturated heterocyclyl, amidinyl, and
Z is H.
   6. An N-terminally modified insulin according to aspect 4, wherein Y and Z are different and
Y is straight chain C1-C4 alkyl, 5- or 6 membered saturated heterocyclyl, and
Z is H.
   7. An N-terminally modified insulin according to aspect 4, wherein Y═Z═C1-C4 alkyl.
   8. An N-terminally modified insulin according to aspect 4, wherein Y and Z are the same and selected from the group consisting of: dimethyl, diethyl, di-n-propyl, di-sec-propyl, di-n-butyl, di-i-butyl.
   9. An N-terminally modified insulin according to aspect 4, wherein Y and Z are the same and selected from dimethyl and diethyl
   10. An N-terminally modified insulin according to aspect 4, wherein Y and Z are the same and dimethyl.
   11. An N-terminally modified insulin according to any one of aspects 1-4, wherein the N-terminal modification is selected from the group consisting of: N,N-di-C1-4 alkyl, N-amidinyl, 4-(N,N-dimethylamino)butanoyl, 3-(1-piperidinyl)propionyl, 3-(N,N-dimethylamino)propionyl, N,N-dimethyl-glycyl and N,N,N-trimethyl-glycyl.
   12. An N-terminally modified insulin according to aspect 11, wherein the N-terminal modification is N,N-di-C1-4 alkyl.
   13. An N-terminally modified insulin according to aspect 12, wherein the N-terminal modification is N,N-dimethyl or N,N-diethyl.
   14. An N-terminally modified insulin according to any one of the previous aspects, wherein the acylated, protease stabilised insulin consists of a protease stabilised insulin as peptide part and a lipophilic substituent attached to the peptide part, wherein the peptide part is human insulin substituted such that at least one hydrophobic amino acid has been substituted with hydrophilic amino acids, and wherein said substitution is within or in close proximity to one or more protease cleavage sites of the insulin.
   15. An N-terminally modified insulin according to aspect 14, wherein the peptide part is human insulin with less than 8 modifications substituted in at least one position selected from the group consisting of: A8H, A14E, A14H, A14D, A21G, desA21, B1E, desB1, B3Q, B3G, B16H, B16E, B25H, B25N, B26G, B26D, B26E, B27G, B27E, B27D, desB27, B28G, B28E, B28D, desB28, and desB30.
   16. An N-terminally modified insulin according to aspect 14, wherein the peptide part is human insulin with less than 8 modifications substituted in at least one position selected from the group consisting of: A14E, A21G, B3Q, B16H, B16E, B25H, B25N, B26G, B27G, desB27, B28G and desB30.
   17. An N-terminally modified insulin according to aspect 14, wherein the peptide part is human insulin with less than 8 modifications substituted in at least two positions selected from the group consisting of: A8H, A14E, A14H, A14D, A21G, desA21, B1E, desB1, B3Q, B3G, B16H, B16E, B25H, B25N, B26G, B26D, B26E, B27G, B27E, B27D, desB27, B28G, B28E, B28D, desB28, and desB30.
   18. An N-terminally modified insulin according to aspect 14, wherein the peptide part is human insulin with less than 8 modifications substituted in at least two positions selected from the group consisting of: A14E, A21G, B3Q, B16H, B16E, B25H, B25N, B26G, B27G, desB27, B28G and desB30
   19. An N-terminally modified insulin according to aspect 14, wherein the peptide part is selected from the group consisting of: A14E, B25H, desB30 human insulin; A14E, B16H, B25H, desB30 human insulin; A14E, B16E, B25H, desB30 human insulin; A14E, desB27, desB30 human insulin; A14E, B16H, desB27, desB30 human insulin; A14E, B25H, B26G, B27G, B28G, desB30 human insulin; B25H, desB30 human insulin and A14E, B25H, desB27, desB30 human insulin.
   20. An N-terminally modified insulin according to aspect 14, wherein the peptide part is selected from the group consisting of: A14E, B25H, desB27, desB30 human insulin; A14E, B16H, B25H, desB27, desB30 human insulin; A14E, desB27, desB30 human insulin; A14E, B16E, B25H, desB27, desB30 human insulin and B25H, desB27, desB30 human insulin.
   21. An N-terminally modified insulin according to aspect 14, wherein the peptide part is selected from the group consisting of: A14E, B25H, desB30 human insulin; A14E, B25H, desB27, desB30 human insulin; A14E, B16H, B25H, desB30 human insulin; A14E, desB27, desB30 human insulin; A14E, B25H, B27E, desB30 human insulin; A14E, A21G, B16H, B25H, desB30 human insulin; A14E, A21G, B25H, desB30 human insulin, A14E, A21G, B25H, desB27, desB30 human insulin, and A14E, A21G, desB27, desB30 human insulin.
   22. An N-terminally modified insulin according to any one of the previous aspects, wherein the acylated, protease stabilised insulin consists of a protease stabilised insulin as peptide part and a lipophilic substituent attached to the peptide part, wherein the lipophilic substituent is a side chain consisting of a fatty acid or a fatty diacid attached to the insulin, optionally via a linker, in an amino acid position of the peptide part.
   23. An N-terminally modified insulin according to aspect 22, wherein the peptide part comprises only one lysine residue and the lipophilic substituent is attached, optionally via a linker, to said lysine residue.
   24. An N-terminally modified insulin according to aspect 22 or 23, wherein the lipophilic substituent has the general formula

Acy-AA1ₙ-AA2ₘ-AA3ₚ- (Formula III),

wherein

n is 0 or an integer in the range from 1 to 3;

m is 0 or an integer in the range from 1 to 10;

p is 0 or an integer in the range from 1 to 10;

Acy is a fatty acid or a fatty diacid comprising from about 8 to about 24 carbon atoms;

AA1 is a neutral linear or cyclic amino acid residue;

AA2 is an acidic amino, acid residue;

AA3 is a neutral, alkyleneglycol-containing amino acid residue;

the order by which AA1, AA2 and AA3 appears in the formula can be interchanged independently; AA2 can occur several times along the formula (e.g., Acy-AA2-AA3₂-AA2-); AA2 can occur independently (=being different) several times along the formula (e.g., Acy-AA2-AA3₂-AA2-); the connections between Acy, AA1, AA2 and/or AA3 are amide (peptide) bonds which, formally, can be obtained by removal of a hydrogen atom or a hydroxyl group (water) from each of Acy, AA1, AA2 and AA3; and attachment to the peptide part can be from the C-terminal end of a AA1, AA2, or AA3 residue in the acyl moiety of the formula (III) or from one of the side chain(s) of an AA2 residue present in the moiety of formula (III).
25. An N-terminally modified insulin, wherein the insulin is an acylated insulin and the N-terminal modification is with one or more N-terminal modification groups that are neutral or negatively charged at physiological pH.
26. An N-terminally modified insulin according to aspect 25, wherein the N-terminally modified insulin consists of a peptide part, a lipophilic substituent and an N-terminal modification group.
27. An N-terminally modified insulin according to aspect 24 or 25, wherein the neutral or negatively charged modification groups at physiological pH are one or two organic substituents which are neutral or negatively charged at physiological pH and are having a MW below 200 g per mol conjugated to the N-terminal of the parent insulin.
28. An N-terminally modified insulin according to any one aspects 25-27, wherein the neutral or negatively charged modification groups at physiological pH are designated Y and Z

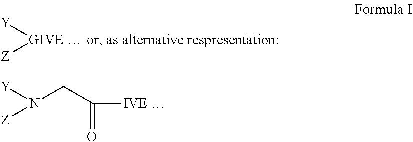

and wherein Y and Z are attached to the N-terminal amino acids of the insulin peptide.
29. An N-terminally modified insulin according to any one of aspects 25-28, wherein the negatively charged N-terminal modification group at physiological pH according to the invention is not malonyl or succinyl.
30. An N-terminally modified insulin according to any one of aspects 25-28, wherein the negatively charged N-terminal modification group at physiological pH according to the invention is not malonyl.
31. An N-terminally modified insulin according to any one of aspects 25-28, wherein the negatively charged N-terminal modification group at physiological pH according to the invention is not succinyl.
32. An N-terminally modified insulin according to any one of aspects ?25-31, wherein the N-terminal modification is selected from the group consisting of: Carbamoyl, thiocarbamoyl, C1-C4 chain acyl groups, oxalyl, glutaryl and diglycolyl.
33. An N-terminally modified insulin according to any one of aspects 25-31, wherein the N-terminal modification is selected from the group consisting of: Carbamoyl, thiocarbamoyl, formyl, acetyl, propionyl, butyryl, pyroglutamyl, oxalyl, glutaryl and diglycolyl.
34. An N-terminally modified insulin according to any one of aspects 25-28, wherein the N-terminal modification is neutral at physiological pH.
35. An N-terminally modified insulin according to any one of aspects 25-28, wherein the N-terminal modification is selected from the group consisting of: Carbamoyl, thiocarbamoyl, formyl, acetyl, propionyl, butyryl, and pyroglutamyl.
36. An N-terminally modified insulin according to any one of aspects 25-31, wherein the N-terminal modification is negatively charged at physiological pH.
37. An N-terminally modified insulin according to any one of aspects 25-28, wherein the N-terminal modification is selected from the group consisting of: oxalyl, glutaryl and diglycolyl.
38. An N-terminally modified insulin according to any one of aspects 25-37, wherein the acylated insulin consists of a peptide part and a lipophilic substituent attached to the peptide part, wherein the peptide part is human insulin, desB30 human insulin, human insulin with less than 8 modifications or desB30 human insulin with less than 8 modifications.
39. An N-terminally modified insulin according to aspect 38, wherein the peptide part is human insulin with less than 8 modifications substituted in at least one position selected from the group consisting of: A8H, A14E, A14H, A14D, A21G, desA21, B1E, desB1, B3Q, B3G, B16H, B16E, B25H, B25N, B26G, B26D, B26E, B27G, B27E, B27D, desB27, B28G, B28E, B28D, desB28, and desB30.
40. An N-terminally modified insulin according to aspect 38, wherein the peptide part is human insulin with less than 8 modifications substituted in at least one position selected from the group consisting of: A14E, A21G, B3Q, B16H, B16E, B25H, B25N, B26G, B27G, desB27, B28G and desB30.
41. An N-terminally modified insulin according to aspect 38, wherein the peptide part is human insulin with less than 8 modifications substituted in at least two positions selected from the group consisting of: A8H, A14E, A14H, A14D, A21G, desA21, B1E, desB1, B3Q, B3G, B16H, B16E, B25H, B25N, B26G, B26D, B26E, B27G, B27E, B27D, desB27, B28G, B28E, B28D, desB28, and desB30.
42. An N-terminally modified insulin according to aspect 38, wherein the peptide part is human insulin with less than 8 modifications substituted in at least two positions selected from the group consisting of: A14E, A21G, B3Q, B16H, B16E, B25H, B25N, B26G, B27G, desB27, B28G and desB30.
43. An N-terminally modified insulin, according to any one of aspects 25-42, wherein the peptide part is human insulin with less than 8 modifications, substituted such that at least one hydrophobic amino acid has been substituted with hydrophilic amino acids, and wherein said substitution is within or in close proximity to one or more protease cleavage sites of the insulin.
44. An N-terminally modified insulin according to any one of aspects 25-43, wherein the peptide part is selected from the group consisting of: A14E, B25H, desB30 human insulin; A14E, B25H, desB27, desB30 human insulin; A14E, B16H, B25H, desB27, desB30 human insulin; A14E, desB27, desB30 human insulin; A14E, B16E, B25H, desB27, desB30 human insulin and B25H, desB27, desB30 human insulin.
45. An N-terminally modified insulin according to any one of aspects 25-43, wherein the peptide part is selected from the group consisting of: A14E, A21G, B25H, desB30 human insulin; A14E, A21G, B16H, B25H, desB30 human insulin; A14E, A21G, B16E, B25H, desB30 human insulin; A14E, A21G, B25H, desB27, desB30 human insulin; A14E, A21G, B25H, desB27, desB30 human insulin; A14E, A21G, B25H, B26G, B27G, B28G, desB30 human insulin; A21G, B25H, desB30 human insulin and A21G, B25N, desB30 human insulin.
46. An N-terminally modified insulin according to any one of aspects 25-43, wherein the peptide part is selected from the group consisting of: A14E, A21G, B25H, desB30 human insulin; A14E, A21G, desB27, desB30 human insulin; A14E, A21G, B16H, B25H, desB30 human insulin; A14E, A21G, B16E, B25H, desB30 human insulin; A14E, A21G, B25H, desB27, desB30 human insulin; A14E, A21G, B25H, desB27, desB30 human insulin; A21G, B25H, desB30 human insulin and A21G, B25N, desB30 human insulin.
47. An N-terminally modified insulin according to any one of aspects 25-43, wherein the peptide part is selected from the group consisting of: A14E, B25H, desB30 human insulin; A14E, B16H, B25H, desB30 human insulin; A14E, B16E, B25H, desB30 human insulin;*A14E, desB27, desB30 human insulin; A14E, B16H, desB27, desB30 human insulin; A14E, B25H, B26G, B27G, B28G, desB30 human insulin; B25H, desB30 human insulin and A14E, B25H, desB27, desB30 human insulin.
48. An N-terminally modified insulin according to any one of aspects 25-47, wherein the acylated, protease stabilised insulin consists of a protease stabilised insulin as peptide part and a lipophilic substituent attached to the peptide part, wherein the lipophilic substituent is a side chain consisting of a fatty acid or a fatty diacid attached to the insulin, optionally via a linker, in an amino acid position of the peptide part.
49. An N-terminally modified insulin according to aspect 48, wherein the peptide part comprises only one lysine residue and the lipophilic substituent is attached, optionally via a linker, to said lysine residue.
50. An N-terminally modified insulin according to aspect 48 or 49, wherein the lipophilic substituent has the general formula

Acy-AA1ₙ-AA2ₘ-AA3ₚ- (Formula III),

wherein

n is 0 or an integer in the range from 1 to 3;

m is 0 or an integer in the range from 1 to 10;

p is 0 or an integer in the range from 1 to 10;

Acy is a fatty acid or a fatty diacid comprising from about 8 to about 24 carbon atoms;

AA1 is a neutral linear or cyclic amino acid residue;

AA2 is an acidic amino acid residue;

AA3 is a neutral, alkyleneglycol-containing amino acid residue;

the order by which AA1, AA2 and AA3 appears in the formula can be interchanged independently; AA2 can occur several times along the formula (e.g., Acy-AA2-AA3₂-AA2-); AA2 can occur independently (=being different) several times along the formula (e.g., Acy-AA2-AA3₂-AA2-); the connections between Acy, AA1, AA2 and/or AA3 are amide (peptide) bonds which, formally, can be obtained by removal of a hydrogen atom or a hydroxyl group (water) from each of Acy, AA1, AA2 and AA3; and attachment to the peptide part can be from the C-terminal end of a AA1, AA2, or AA3 residue in the acyl moiety of the formula (III) or from one of the side chain(s) of an AA2 residue present in the moiety of formula (III).
51. A N-terminally modified insulin according to any one of the preceeding claims, which is selected from the group consisting of:
A1(N^{α},N^{α}-Dimethyl), A14E, B1(N^{α},N^{α}-dimethyl), B25H, B29K(N^{ε}octadecanedioyl-gGlu-2xOEG), desB30 human insulin
A1(N^{α},N^{α}-Diethyl), A14E, B1(N^{α},N^{α}-diethyl), B25H, B29K(N^{ε}Octadecanedioyl-gGlu-2xOEG), desB30 human insulin
A1(N^{α},N^{α}-Dimethyl), A14E, B1(N^{α},N^{α}-dimethyl), B16H, B25H, B29K(N^{ε}hexadecanedioyl-gGlu), desB30 human insulin
A1(N^{α},N^{α}-Dimethyl), A14E, B1(N^{α},N^{α}-dimethyl), B25H, desB27, B29K(N^{ε}octadecanedioyl-gGlu), desB30 human insulin
A1(N^{α},N^{α}-Dimethyl), A14E, B1(N^{α},N^{α}-dimethyl), B25H, desB27, B29K(N^{ε}octadecanedioyl-gGlu-2xOEG), desB30 human insulin
A1(N^{α},N^{α}-Dimethyl), A14E, B1(N^{α},N^{α}-dimethyl), desB27, B29K(N^{ε}octadecanedioyl-gGlu-2xOEG), desB30 human insulin
A1(N^{α},N^{α}-Dimethyl), A14E, B1(N^{α},N^{α}-dimethyl), B16H, B25H, B29K(N^{ε}eicosanedioyl-gGlu-2xOEG), desB30 human insulin
A1G(N^{α},N^{α}-Dimethyl), A14E, B1F(N^{α},N^{α}-dimethyl), B25H, desB27, B29K(N^{ε}hexadecanedioyl-gGlu), desB30 human insulin
A1G(N^{α},N^{α}-Dimethyl), A14E, B1F(N(alpha),N(N^{α},N^{α}-dimethyl), B25H, desB27, B29K(N^{ε}hexadecanedioyl-gGlu-2xOEG), desB30 human insulin
A1(N^{α},N^{α}-Dimethyl), A14E, B1(N^{α},N^{α}-dimethyl), desB27, B29K(N^{ε}octadecanedioyl-gGlu), desB30 human insulin
A1(N^{α},N^{α}-Dimethyl), A14E, B1(N^{α},N^{α}-dimethyl), B25H, B29K(N^{ε}octadecanedioyl-gGlu), desB30 human insulin
A1(N^{α}Carbamoyl), A14E, B1(W B25H, B29K(N^{ε}-octadecanedioyl-gGlu-2xOEG), desB30 human insulin
A1(N^{α}Carbamoyl), A14E, B1(N^{α}Carbamoyl), B25H, B29K(N^{ε}hexadecanedioyl-gGlu), desB30 human insulin
A1(N^{α}Carbamoyl), A14E, B1(N^{α}Carbamoyl), B25H, B29K(N^{ε}eicosanedioyl-gGlu), desB30 human insulin
A1(N^{α}Carbamoyl), A14E, B1(N^{α}Carbamoyl), B25H, B29K(N^{ε}eicosanedioyl-gGlu-2xOEG), desB30 human insulin
A1(N^{α}Carbamoyl), A14E, B1(N^{α}Carbamoyl), B16H, B25H, B29K(N^{ε}eicosanedioyl-gGlu-2xOEG); desB30 human insulin
A1(N^{α}Carbamoyl), A14E, B1(N^{α}Carbamoyl), B25H, desB27, B29K(N^{ε}octadecandioyl-gGlu), desB30 human insulin
A1(N^{α}Carbamoyl), A14E, B1(N^{α}Carbamoyl), B25H, desB27, B29K(N^{ε}octadecandioyl-gGlu-2xOEG), desB30 human insulin
A1G(N(alpha)carbamoyl), A14E, B1F(N(alpha)carbamoyl), desB27, B29K(N(eps)hexadecanedioyl-gGlu), desB30 human insulin
A1G(N(alpha)carbamoyl), A14E, B1F(N(alpha)carbamoyl), desB27, B29K(Neps)hexadecanedioyl-gGlu-2xOEG), desB30 human insulin
A1G(N(alpha)carbamoyl), A14E, B1F(N(alpha)carbamoyl), desB27, B29K(Neps)-eicosanedioyl-gGlu), desB30 human insulin
A1G(N^{α}carbamoyl), A14E, B1F(N^{α}carbamoyl), B16H, desB27, B29K(Neps)-eicosanedioyl-gGlu-2xOEG), desB30 human insulin
A1(N^{α}Carbamoyl), A14E, B1(N^{α}Carbamoyl), desB27, B29K(N^{ε}octadecanedioyl-gGlu), desB30 human insulin
A1(N^{α}Carbamoyl), A14E, B1(N^{α}Carbamoyl), B16H, B25H, B29K(N^{ε}eicosanedioyl-gGlU), desB30 human insulin
A1(N^{α}Carbamoyl), A14E, B1(N^{α}Carbamoyl), desB27, B29K(N^{ε}octadecanedioyl-gGlu-2xOEG), desB30 human insulin
A1(N^{α}Carbamoyl), A14E, B1(N^{α}carbamoyl), B25H, B29K(N^{ε}octadecanedioyl-gGlu), desB30 human insulin
A1(N^{α}Carbamoyl), A14E, B1(N^{α}Carbamoyl), B16H, B25H, B29K(N^{ε}eicosanedioyl-gGlu), desB30 human insulin
A1G(N^{α}carbamoyl), A14E, B1F(N^{α}carbamoyl), B25H, desB27, B29K(N^{ε}eicosanedioyl-gGlu-2xOEG), desB30 human insulin
A1G(N^{α}carbamoyl), A14E, B1F(N^{α}carbamoyl), desB27, B29K(N^{ε}eicosanedioyl-gGlu-2xOEG), desB30 human insulin
A1G(N^{α}carbamoyl), A14E, B1F(N^{α}carbamoyl), B16H, desB27, B29K(N^{ε}eicosanedioyl-gGlu-2xOEG), desB30 human insulin
A1G(N^{α}thiocarbamoyl), A14E, B1F(NN^{α}thiocarbamoyl), B25H, desB27, B29K(N^{ε}-octadecanedioyl-gGlu-2xOEG), desB30 human insulin

A1(N^{α}Acetyl), A14E, B1(N^{α}Acetyl), B25H, B29K(N^{ε}hexadecanedioyl-gGlu), desB30 human insulin
A1(N^{α}Acetyl), A14E, B1(N^{α}Acetyl), B25H, desB27, B29K(N^{ε}octadecanedioyl-gGlu), desB30 human insulin
A1(N^{α}Acetyl), A14E, B1(N^{α}Acetyl), B25H, B29K(N^{ε}octadecandioyl-gGlu-2xOEG), desB30 human insulin
A1(N^{α}Dimethylglycyl), A14E, B1(N^{α}Dimethylglycyl), B25H, B29K(N^{ε}octadecanedioyl-gGlu-2xOEG), desB30 human insulin
A1(N^{α}3-(N,N-Dimethylamino)propionyl), A14E, B1 (N^{α}3-(N,N-dimethylamino)propionyl), B25H, B29K(N^{ε}octadecanedioyl-gGlu-2xOEG), desB30 human insulin
A1(N^{α}4-(N,N-Dimethylamino)butanoyl), A14E, B1 (N^{α}4-(N,N-dimethylamino)butanoyl), B25H, B29K(N^{ε}-octadecanedioyl-gGlu-2xOEG), desB30 human insulin
A1(N^{α}3-(1-Piperidinyl)propionyl), A14E, B1(N^{α}3-(1-piperidinyl)propionyl), B25H, B29K(N^{ε}-octadecanedioyl-gGlu-2xOEG), desB30 human insulin
A1(N^{α}Dimethylglycyl), A14E, B1(N^{α}Dimethylglycyl), B25H, desB27, B29K(N^{ε}octadecanedioyl-gGlu), desB30 human insulin
A1G(N^{α}acetyl), A14E, B1F(N^{α}acetyl), B25H, desB27, B29K(N^{ε}octadecanedioyl-gGlu-2xOEG), desB30 human insulin
A1G(N^{α}2-Picolyl), A14E, B1F(N^{α}2-Picolyl), B25H, desB27, B29K(N(eps)octadecanedioyl-gGlu-2xOEG), desB30 human insulin
A1(N^{α}Acetyl), A14E, B1(N^{α}Acetyl), B25H, B29K(N^{ε}eicosanedioyl-gGlu), desB30 human insulin
A1(N^{α}Acetyl), A14E, B1(N^{α}Acetyl), B25H, B29K(N^{ε}eicosanedioyl-gGlu-2xOEG), desB30 human insulin
A1(N^{α}Acetyl), A14E, B1(N^{α}Acetyl), B16H, B25H, B29K(N^{ε}eicosanedioyl-gGlu-2xOEG), desB30 human insulin
A1(N^{α}Acetyl), A14E, B1(N^{α}Acetyl), B16H, B25H, B29K(N^{ε}eicosanedioyl-gGlu), desB30 human insulin
A1(N^{α}Dimethylglycyl), A14E, B1(N^{α}Dimethylglycyl), B16H, B25H, B29K(N^{ε}hexadecanedioyl-gGlu), desB30 human insulin
A1(N^{α}Trimethyl), A14E, B-1(N^{α}Trimethyl), B25H, B29K(N^{ε}octadecanedioyl-gGlu-2xOEG), desB30 human insulin

A1(N^{α}Acetyl), A14E, B1(N^{α}Acetyl), desB27, B29K(N^{ε}octadecanedioyl-gGlu), desB30 human insulin
A1(N^{α}Acetyl), A14E, B1(N²Acetyl), desB27, B29K(N^{ε}octadecanedioyl-gGlu-2xOEG), desB30 human insulin
A1(N^{α}Acetyl), A14E, B1(N^{α}Acetyl), B25H, B29K(N^{ε}octadecanedioyl-gGlu), desB30 human insulin
A1G(N^{α}Acetyl), A14E, B1F(N^{α}Acetyl), desB27, B29K(N^{ε}eicosanedioyl-gGlu), desB30 human insulin
A1G(N^{α}Acetyl), A14E, B1F(N^{α}Acetyl), desB27, B29K(N^{ε}eicosanedioyl-gGlu-2xOEG), desB30 human insulin
A1G(N^{α}Acetyl), A14E, B1F(N^{α}Acetyl), B25H, desB27, B29K(N^{ε}eicosanedioyl-gGlu-2xOEG), desB30 human insulin
A1(N′Succinyl), A14E, B1(N^{α}succinyl), B25H, desB27, B29K(N^{ε}octadecanedioyl-gGlu-2xOEG), desB30 human insulin
A1(N^{α}Succinyl), A14E, B1(N^{α}succinyl), B25H, B29K(N^{ε}octadecanedioyl-gGlu-2xOEG), desB30 human insulin
A1(N^{α}Succinyl), A14E, B1(N^{α}succinyl), desB27, B29K(N^{ε}octadecanedioyl-gGlu-2xOEG), desB30 human insulin
A1(N^{α}Glutaryl), A14E, B1(N^{α}glutaryl), B25H, B29K(N^{ε}octadecanedioyl-gGlu-2xOEG), desB30 human insulin
A1(N^{α}Glutaryl), A14E, B1(N^{α}glutaryl), desB27, B29K(N^{ε}octadecanedioyl-gGlu-2xOEG), desB30 human insulin
A1(N^{α}Diglycolyl), A14E, B1 (N^{α}diglycolyl), B25H, desB27, B29K(N^{ε}octadecanedioyl-gGlu-2xOEG), desB30 human insulin
A1(N^{α}Glutaryl), A14E, B1(N^{α}glutaryl), B25H, desB27, B29K(N^{ε}octadecanedioyl-gGlu-2xOEG), desB30 human insulin
A1(N^{α}Succinyl), A14E, B1(N^{α}succinyl), desB27, B29K(N^{ε}octadecanedioyl-gGlu), desB30 human insulin
A1(N^{α}Succinyl), A14E, B1(N^{α}succinyl), B25H, desB27, B29K(N^{ε}eicosanedioyl-gGlu-2xOEG), desB30 human insulin
A1(N^{α}Succinyl), A14E, B1(N^{α}succinyl), desB27, B29K(N^{ε}eicosanedioyl-gGlu-2xOEG), desB30 human insulin
A1(N^{α}Succinyl), A14E, B1(N^{α}succinyl), B16H, desB27, B29K(N^{ε}eicosanedioyl-gGlu-2xOEG), desB30 human insulin
A1(N^{α}Succinyl), A14E, B1(N^{α}succinyl), B25H, B29K(N^{ε}eicosanedioyl-gGlu-2xOEG), desB30 human insulin
A1(N^{α}Succinyl), A14E, B1(N^{α}succinyl), desB27, B29K(N^{ε}eicosanedioyl-gGlu), desB30 human insulin
A1(N^{α}Glutaryl), A14E, B1(N^{α}glutaryl), desB27, B29K(N^{ε}eicosanedioyl-gGlu), desB30 human insulin
A1(N^{α}Glutaryl), A14E, B1(N^{α}glutaryl), desB27, B29K(N^{ε}eicosanedioyl-gGlu-2xOEG), desB30 human insulin
A1(N^{α}Glutaryl), A14E, B1(N^{α}glutaryl), B25H, desB27, B29K(N^{ε}eicosanedioyl-gGlu-2xOEG), desB30 human insulin
A1(N^{α}Glutaryl), A14E, B1(N^{α}glutaryl), desB27, B29K(N^{ε}eicosanedioyl-gGlu-2xOEG), desB30 human insulin
A1(N^{α}Glutaryl), A14E, B1(N^{α}glutaryl), B25H, B29K(N^{ε}eicosanedioyl-gGlu-2xOEG), desB30 human insulin.
   52. A N-terminally modified insulin according to any one of the preceeding claims, which is selected from the group consisting of:
A1(N^{α},N^{α}-Dimethyl), A14E, B1(N^{α},N^{α}-dimethyl), B25H, B29K(N^{ε}octadecanedioyl-gGlu-2xOEG), desB30 human insulin
A1(N^{α},N^{α}-Diethyl), A14E, B1(N^{α},N^{α}-diethyl), B25H, B29K(N^{ε}Octadecanedioyl-gGlu-2xOEG), desB30 human insulin
A1(N^{α},N^{α}-Dimethyl), A14E, B1(N^{α},N^{α}-dimethyl), B16H, B25H, B29K(N^{ε}hexadecanedloyl-gGlu), desB30 human insulin
A1(N^{α},N^{α}-Dimethyl), A14E, B1(N^{α},N^{α}-dimethyl), B25H, desB27, B29K(N^{ε}octadecanedioyl-gGlu), desB30 human insulin
A1(N^{α},N^{α}-Dimethyl), A14E, B1(N^{α},N^{α}-dimethyl), B25H, desB27, B29K(N^{ε}octadecanedioyl-gGlu-2xOEG), desB30 human insulin
A1(N^{α},N^{α}-Dimethyl), A14E, B1(N^{α},N^{α}-dimethyl), desB27, B29K(N^{ε}octadecanedioyl-gGlu-2xOEG), desB30 human insulin
A1(N^{α},N^{α}-Dimethyl), A14E, B1(N^{α},N^{α}-dimethyl), B16H, B25H, B29K(N^{ε}eicosanedioyl-gGlu-2xOEG), desB30 human insulin
A1G(N^{α},N^{α}-Dimethyl), A14E, B1F(N^{α}, N^{α}-dimethyl), B25H, desB27, B29K(N^{ε}hexadecanedioyl-gGlu), desB30 human insulin
A1G(N^{α}, N^{α}-Dimethyl), A14E, B1F(N(alpha),N(N^{α},N^{α}-dimethyl), B25H, desB27, B29K(N^{ε}hexadecanedioyl-gGlu-2xOEG), desB30 human insulin
A1(N^{α},N^{α}-Dimethyl), A14E, B1(N^{α},N^{α}-dimethyl), desB27, B29K(N^{ε}octadecanedioyl-gGlu), desB30 human insulin
A1(N^{α},N^{α}-Dimethyl), A14E, B1(N^{α},N^{α}-dimethyl), B25H, B29K(N^{ε}octadecanedioyl-gGlu), desB30 human insulin
A1(N^{α}Carbamoyl), A14E, B1(N^{α}Carbamoyl), B25H, B29K(N^{ε}octadecanedioyl-gGlu-2xOEG), desB30 human insulin
A1(N^{α}Carbamoyl), A14E, B1(N^{α}-Carbamoyl), B25H, B29K(N^{ε}hexadecanedioyl-gGlu), desB30 human insulin
A1(N^{α}Carbamoyl), A14E, B1(N^{α}Carbamoyl), B25H, B29K(N^{ε}eicosanedioyl-gGlu), desB30 human insulin
A1(N^{α}Carbamoyl), A14E, B1(N^{α}Carbamoyl), B25H, B29K(N^{ε}eicosanedioyl-gGlu-2xOEG), desB30 human insulin
A1(N^{α}Carbamoyl), A14E, B1(N^{α}Carbamoyl), B16H, B25H, B29K(N^{ε}eicosanedioyl-gGlu-2xOEG), desB30 human insulin
A1(N^{α}Carbamoyl), A14E, B1(N^{α}Carbamoyl), B25H, desB27, B29K(N^{ε}octadecandioyl-gGlu), desB30 human insulin
A1(N^{α}Carbamoyl), A14E, B1(N^{α}Carbamoyl), B25H, desB27, B29K(N^{ε}octadecandioyl-gGlu-2xOEG), desB30 human insulin
A1G(N(alpha)carbamoyl), A14E, B1F(N(alpha)carbamoyl), desB27, B29K(N(eps)hexadecanedioyl-gGlu), desB30 human insulin
A1G(N(alpha)carbamoyl), A14E, B1F(N(alpha)carbamoyl), desB27, B29K(Neps)hexadecanedioyl-gGlu-2xOEG), desB30 human insulin
A1G(N(alpha)carbamoyl), A14E, B1F(N(alpha)carbamoyl), desB27, B29K(Neps)-eicosanedioyl-gGlu), desB30 human insulin
A1G(N^{α}carbamoyl), A14E, B1F(N^{α}carbamoyl), B16H, desB27, B29K(Neps)-eicosanedioyl-gGlu-2xOEG), desB30 human insulin
A1(N^{α}Carbamoyl), A14E, B1(N^{α}Carbamoyl), desB27, B29K(N^{ε}octadecanedioyl-gGlu), desB30 human insulin
A1(N^{α}Carbamoyl), A14E, B1(N^{α}Carbamoyl), B16H, B25H, B29K(N^{ε}eicosanedioyl-gGlu), desB30 human insulin
A1(N^{α}Carbamoyl), A14E, B1(N^{α}Carbamoyl), desB27, B29K(N^{ε}octadecanedioyl-gGlu-2xOEG), desB30 human insulin
A1(N^{α}Carbamoyl), A14E, B1(N^{α}carbamoyl), B25H, B29K(N^{ε}octadecanedioyl-gGlu), desB30 human insulin
A1(N^{α}Carbamoyl), A14E, B1(N^{α}Carbamoyl), B16H, B25H, B29K(N^{ε}eicosanedioyl-gGlu), desB30 human insulin
A1G(N^{α}carbamoyl), A14E, B1F(N^{α}carbamoyl), B25H, desB27, B29K(N^{ε}eicosanedioyl-gGlu-2xOEG), desB30 human insulin
A1G(N^{α}carbamoyl), A14E, B1F(N^{α}carbamoyl), desB27, B29K(N^{ε}eicosanedioyl-gGlu-2xOEG), desB30 human insulin
A1G(N^{α}carbamoyl), A14E, B1F(N^{α}carbamoyl), B16H, desB27, B29K(N^{ε}eicosanedioyl-gGlu-2xOEG), desB30 human insulin
A1G(N^{α}thiocarbamoyl), A14E, B1F(NN^{ε}thiocarbamoyl), B25H, desB27, B29K(N^{ε}octadecanedioyl-gGlu-2xOEG), desB30 human insulin
A1(N^{α}Acetyl), A14E, B1(N²Acetyl), B25H, B29K(N^{ε}hexadecanedioyl-gGlu), desB30 human insulin
A1(N^{α}Acetyl), A14E, B1(N^{α}Acetyl), B25H, desB27, B29K(N^{ε}octadecanedioyl-gGlu), desB30 human insulin
A1(N^{α}Acetyl), A14E, B1(N²Acetyl), B25H, B29K(N^{ε}octadecandioyl-gGlu-2xOEG), desB30 human insulin
A1(N^{α}Dimethylglycyl), A14E, B1(N^{α}Dimethylglycyl), B25H, B29K(N^{ε}octadecanedioyl-gGlu-2xOEG), desB30 human insulin
A1(N^{α}3-(N,N-Dimethylamino)propionyl), A14E, B1(N^{α}3-(N,N-dimethylamino)propionyl), B25H, B29K(N^{ε}octadecanedioyl-gGlu-2xOEG), desB30 human insulin
A1(N^{α}4-(N,N-Dimethylamino)butanoyl), A14E, B1(N^{α}4-(N,N-dimethylamino)butanoyl), B25H, B29K(N^{ε}octadecanedioyl-gGlu-2xOEG), desB30 human insulin
A1(N^{α}3-(1-Piperidinyl)propionyl), A14E, B1(N^{α}3-(1-piperidinyl)propionyl), B25H, B29K(N^{ε}octadecanedioyl-gGlu-2xOEG), desB30 human insulin
A1(N^{α}Dimethylglycyl), A14E, B1(N^{α}Dimethylglycyl), B25H, desB27, B29K(N^{ε}octadecanedioyl-gGlu), desB30 human insulin
A1G(N^{α}acetyl), A14E, B1F(N^{α}acetyl), B25H, desB27, B29K(N^{ε}octadecanedioyl-gGlu-2xOEG), desB30 human insulin
A1G(N^{α}2-Picolyl), A14E, B1F(N^{α}2-Picolyl), B25H, desB27, B29K(N(eps)octadecanedioyl-gGlu-2xOEG), desB30 human insulin
A1(N^{α}Acetyl), A14E, B1(N^{α}Acetyl), B25H, B29K(N^{ε}eicosanedioyl-gGlu), desB30 human insulin
A1(N^{α}Acetyl), A14E, B1(N^{α}Acetyl), B25H, B29K(N^{ε}eicosanedioyl-gGlu-2xOEG), desB30 human insulin
A1(N^{α}Acetyl), A14E, B1(N^{α}Acetyl), B16H, B25H, B29K(N^{ε}eicosanedioyl-gGlu-2xOEG), desB30 human insulin
A1(N^{α}Acetyl), A14E, B1(N^{α}Acetyl), B16H, B25H, B29K(N^{ε}eicosanedioyl-gGlu), desB30 human insulin
A1(N^{α}Dimethylglycyl), A14E, B1(N^{α}Dimethylglycyl), B16H, B25H, B29K(N^{ε}hexadecanedioyl-gGlu), desB30 human insulin
A-1(N^{α}Trimethyl), A14E, B-1(N^{α}Trimethyl), B25H, B29K(N^{ε}octadecanedioyl-gGlu-2xOEG), desB30 human insulin
A1(N^{α}Acetyl), A14E, B1(N^{α}Acetyl), desB27, B29K(N^{ε}octadecanedioyl-gGlu), desB30 human insulin
A1(N^{α}Acetyl), A14E, B1(N^{α}Acetyl), desB27, B29K(N^{ε}octadecanedioyl-gGlu-2xOEG), desB30 human insulin
A1(N^{α}Acetyl), A14E, B1(N^{α}Acetyl), B25H, B29K(N^{ε}octadecanedioyl-gGlu), desB30 human insulin
A1G(N^{α}Acetyl), A14E, B1F(N^{α}Acetyl), desB27, B29K(N^{ε}eicosanedioyl-gGlu), desB30 human insulin
A1G(N^{α}Acetyl), A14E, B1F(N^{α}Acetyl), desB27, B29K(N^{ε}eicosanedioyl-gGlu-2xOEG), desB30 human insulin
A1G(N^{α}Acetyl), A14E, B1F(N^{α}Acetyl), B25H, desB27, B29K(N^{ε}eicosanedioyl-gGlu-2xOEG), desB30 human insulin
A1(N^{α}Glutaryl), A14E, B1(N^{α}glutaryl), B25H, B29K(N^{ε}octadecanedioyl-gGlu-2xOEG), desB30 human insulin
A1(N^{α}Glutaryl), A14E, B1(N^{α}glutaryl), desB27, B29K(N^{ε}octadecanedioyl-gGlu-2xOEG), desB30 human insulin
A1(N^{α}Diglycolyl), A14E, B1(N^{α}diglycolyl), B25H, desB27, B29K(N^{ε}octadecanedioyl-gGlu-2xOEG), desB30 human insulin
A1(N^{α}Glutaryl), A14E, B1(N^{α}glutaryl), B25H, desB27, B29K(N^{ε}octadecanedioyl-gGlu-2xOEG), desB30 human insulin
A1(N^{α}Glutaryl), A14E, B1(N^{α}glutaryl), desB27, B29K(N^{ε}eicosanedioyl-gGlu), desB30 human insulin
A1(N^{α}Glutaryl), A14E, B1(N^{α}glutaryl), desB27, B29K(N^{ε}eicosanedioyl-gGlu-2xOEG), desB30 human insulin
A1(N^{α}Glutaryl), A14E, B1(N^{α}glutaryl), B25H, desB27, B29K(N^{ε}eicosanedioyl-gGlu-2xOEG), desB30 human insulin
A1(N^{α}Glutaryl), A14E, B1(N^{α}glutaryl), desB27, B29K(N^{ε}eicosanedioyl-gGlu-2xOEG), desB30 human insulin
A1(N^{α}Glutaryl), A14E, B1(N^{α}glutaryl), B25H, B29K(N^{ε}eicosanedioyl-gGlu-2xOEG), desB30 human insulin.
   53. An N-terminally modified insulin according to any one of the preceeding claims, which is selected from the group consisting of:
A1(Nα,Nα-Dimethyl), A14E, B1(Nα,Nα-dimethyl), B25H, B29K(Nεoctadecanedioyl-gGlu-2xOEG), desB30 human insulin
A1(Nα,Nα-Diethyl), A14E, B1(Nα,Nα-diethyl), B25H, B29K(NεOctadecanedioyl-gGlu-2xOEG), desB30 human insulin
A1(Nα,Nα-Dimethyl), A14E, B1(Nα,Nα-dimethyl), B25H, B29K(Nεoctadecanedioyl-gGlu), desB27, desB30 human insulin
A1(Nα,Nα-Dimethyl), A14E, B1(Nα,Nα-dimethyl), B16H, B25H, B29K(Nεhexadecanedioyl-gGlu), desB30 human insulin
A1(NαCarbamoyl), A14E, B1(NαCarbamoyl), B25H, B29K(Nεoctadecanedioyl-gGlu-2xOEG), desB30 human insulin
A1 (NαCarbamoyl), A14E, B1(NαCarbamoyl), B25H, B29K(Nεhexadecanedioyl-gGlu), desB30 human insulin
A1(NαCarbamoyl), A14E, B1(NαCarbamoyl), B25H, B29K(Nεeicosanedioyl-gGlu), desB30 human insulin
A1(NαCarbamoyl), A14E, B1(NαCarbamoyl), B16H, B25H, B29K(Nεeicosanedioyl-gGlu), desB30 human insulin
A1(NαCarbamoyl), A14E, B1(NαCarbamoyl), B25H, B29K(Nεeicosanedioyl-gGlu-2xOEG), desB30 human insulin
A1(NαCarbamoyl), A14E, B1(NαCarbamoyl), B16H, B25H, B29K(Nεeicosanedioyl-gGlu-2xOEG), desB30 human insulin
A1(NαAcetyl), A14E, B1(NαAcetyl), B25H, B29K(Nεhexadecanedioyl-gGlu), desB30 human insulin
A1(NαAcetyl), A14E, B1(NαAcetyl), B25H, B29K(Nεeicosanedioyl-gGlu), desB30 human insulin
A1(NαAcetyl), A14E, B1(NαAcetyl), B25H, B29K(Nεeicosanedioyl-gGlu-2xOEG), desB30 human insulin
A1(NαAcetyl), A14E, B1(NαAcetyl), B16H, B25H, B29K(Nεeicosanedioyl-gGlu-2xOEG), desB30 human insulin
A1(NαAcetyl), A14E, B1(NαAcetyl), B16H, B25H, B29K(Nεeicosanedioyl-gGlu), desB30 human insulin
A1(NαDimethylglycyl), A14E, B1(NαDimethylglycyl), B25H, B29K(Nεoctadecanedioyl-gGlu-2xOEG), desB30 human insulin
A1(NαDimethylglycyl), A14E, B1(NαDimethylglycyl), B16H, B25H, B29K(Nεhexadecanedioyl-gGlu), desB30 human insulin
A1(NαTrimethyl), A14E, B-1(NαTrimethyl), B25H, B29K(Nεoctadecanedioyl-gGlu-2xOEG), desB30 human insulin
A1(Nα,Nα-Dimethyl), A14E, B1(Nα,Nα-dimethyl), B25H, B29K(Nεoctadecanedioyl-gGlu-2xOEG), desB27, desB30 human insulin
A1(Nα,Nα-Dmethyl), A14E, B1(Nα,Nα-dimethyl), B16H, B25H, B29K(Nεeicosanedioyl-gGlu-2xOEG), desB30 human insulin
A1(N-carbamoyl), A14E,B1 (N-carbamoyl), B25H, desB27, B29K(Nε(octadecandioyl-gGlu), desB30 human insulin
A1(N^{α}Carbamoyl), A14E, B1(N^{α}Carbamoyl), B25H, desB27, B29K(N^{ε}octadecandioyl-gGlu-2xOEG), desB30 human insulin
A1(N-Acetyl), A14E, B1(N-acetyl), B25H, desB27, B29K(N-(eps)-(octadecandioyl-gGlu), desB30 human insulin
A1(NαAcetyl), A14E, B1(NαAcetyl), B25H, B29K(Nεoctadecandioyl-gGlu-2xOEG), desB30 human insulin
A1(N-Dimethylaminopropionyl,A14E,B1(N-dimethylaminopropionyl, B25H, B29K(N(eps) octadecanedioyl-gGlu-2xOEG), desB30 human insulin
A1-(N-Dimethylaminobutanoyl), A14E,B1-(N-dimethylaminobutanoyl), B25H, B29K(N(eps)octadecanedioyl-gGlu-2xOEG), desB30 human insulin
A1-(N-(3-(1-Piperidinylpropionyl))), A14E,B1-(N-(3-(1-piperidinylpropionyl))), B25H, B29K(N(eps)octadecanedioyl-gGlu-2xOEG), desB30 human insulin
A1(NαDimethylglycyl), A14E, B1(NαDimethylglycyl), B25H, B29K(Nεoctadecanedioyl-gGlu-2xOEG), desB30 human insulin
A1(NαDimethylglycyl), A14E, B1(NαDimethylglycyl), B25H, desB27, B29K(N-(eps)-(octadecandioyl-gGlu), desB30 human insulin
A1(NαAcetyl), A14E, B1(NαAcetyl), B25H, B29K(Nεoctadecandioyl-gGlu-2xOEG),des B27, desB30 human insulin
A1(N^{α},N^{α}-Dimethyl), A14E, B1(N^{α},N^{α}-dimethyl), desB27, B29K(N^{ε}octadecanedioyl-gGlu-2xOEG), desB30 human insulin
A1(N^{α},N^{α}-Dimethyl), A14E, B1(N^{α},N^{α}-dimethyl), desB27, B29K(N^{ε}octadecanedioyl-gGlu), desB30 human insulin

A1(N^{α},N^{α}-Dimethyl), A14E, B1(N^{α},N^{α}-dimethyl), B25H, B29K(N^{α}octadecanedioyl-gGlu), desB30 human insulin
A1 (N^{α}Carbamoyl), A14E, B1(N^{α}Carbamoyl), desB27, B29K(N^{ε}octadecanedioyl-gGlu), desB30 human insulin
A1(N^{α}Carbamoyl), A14E, B1(N^{α}Carbamoyl), desB27, B29K(N^{ε}octadecanedioyl-gGlu-2xOEG), desB30 human insulin
A1 (N^{α}-Carbamoyl), A14E, B1(N^{α}carbamoyl), B25H, B29K(N^{ε}octadecanedioyl-gGlu), desB30 human insulin
A1(N^{α}Acetyl), A14E, B1(N^{α}Acetyl), desB27, B29K(N^{ε}octadecanedioyl-gGlu), desB30 human insulin
A1(N^{α}Acetyl), A14E, B1(N^{α}Acetyl), B25H, B29K(N^{ε}octadecanedioyl-gGlu), desB30 human insulin
   54. A N-terminally modified insulin according to any of the preceding, possible aspects which is any one of the compounds mentioned specifically in the above specification.
   55. A pharmaceutical composition comprising an N-terminally modified insulin according to any one of the preceding aspects.
   56. A pharmaceutical composition according to aspect 55, which is an oral pharmaceutical composition.
   57. An oral pharmaceutical composition comprising one or more lipids and an N-terminally modified insulin.
   58. An N-terminally modified insulin according to aspect 57, wherein the N-terminally modified insulin consists of a peptide part, an N-terminal modification group and optionally a lipophilic substituent.
   59. An N-terminally modified insulin according to aspect 57, wherein the N-terminally modified insulin consists of a peptide part, an N-terminal modification group and a lipophilic substituent.
   60. An oral pharmaceutical composition according to any one of aspects 57-59, which is anhydrous.
   61. An oral pharmaceutical composition according to any one of aspects 57-60, wherein the lipids are selected from the group consisting of: Glycerol mono-caprylate (such as e.g. Rylo MG08 Pharma) and Glycerol mono-caprate (such as e.g. Rylo MG10 Pharma from Danisco). In another aspect the lipid is selected from the group consisting of: propyleneglycol caprylate (such as e.g. Capmul PG8 from Abitec or Capryol PGMC, or Capryol 90 from Gattefosse).
   62. An oral pharmaceutical composition according to any one of aspects 57-61, which is a solid or semi-solid pharmaceutical composition comprising an N-terminally modified insulin (a), at least one polar organic solvent (b) for the N-terminally modified insulin, at least one surfactant (c), at least one lipophilic component (d), and optionally at least one solid hydrophilic component (e), wherein said pharmaceutical composition is spontaneously dispersible.
   63. An oral pharmaceutical composition according to any one of aspects 57-61, which is a water-free liquid pharmaceutical composition comprising an N-terminally modified insulin (a), at least one polar organic solvent (b) for the N-terminally modified insulin, at least one lipophilic component (c), and optionally at least one surfactant (d), wherein the pharmaceutical composition is in the form of a clear solution.
   64. An oral pharmaceutical composition according to any one of aspects 57-63, wherein the surfactant is a non-ionic surfactant.
   65. An oral pharmaceutical composition according to any one of aspects 57-63, wherein the surfactant is a solid surfactant selected from the group consisting of a poloxamer and a mixture of poloxamers such as Pluronic F-127 or Pluronic F-68.
   66. An oral pharmaceutical composition according to any one of aspects 57-65, wherein the lipophilic component is a mono-di-glyceride.
   67. An oral pharmaceutical composition according to any one of aspects 57-66, wherein the lipophilic component is chosen such that a solution is obtained when the lipophilic component is mixed with propylene glycol.
   68. An oral pharmaceutical composition according to any one of aspects 57-67, wherein the lipophilic component is a mono- and/or di-glyceride or propylene glycol caprylate.
   69. An oral pharmaceutical composition according to any one of aspects 57-61, which is a liquid pharmaceutical composition comprising at least one N-terminally modified insulin, at least one polar organic solvent and at least two non-ionic surfactants with HLB above 10, wherein the composition does not contain oil or any other lipid component or surfactant with an HLB below 7.
   70. An oral pharmaceutical composition according to any one of aspects 57-69, wherein the composition forms a micro- or nanoemulsion after dilution in an aqueous medium.
   71. An oral pharmaceutical composition according to any one of aspects 57-70, wherein the organic solvent is selected from the group consisting of polyols.
   72. An oral pharmaceutical composition according to any one of aspects 57-71, wherein the organic solvent is selected from the group consisting of propylene glycol, glycerol and mixtures thereof.
   73. An oral pharmaceutical composition according to any one of aspects 57-72, wherein the organic solvent is propylene glycol.
   74. An oral pharmaceutical composition according to any one of aspects 69-73, wherein one or more of said non-ionic surfactants comprise a medium chain fatty acid group such as C8 fatty acids (caprylates), C10 fatty acids (caprates) or C12 fatty acids (laurates)
   75. An oral pharmaceutical composition according to any one of aspects 69-73, wherein one or more of said non-ionic surfactants are selected from the group consisting of Labrasol (also named Caprylocaproyl Macrogolglycerides), Tween 20 (also named Polysorbate 20 or Polyethylene glycol sorbitan monolaurate), Tween 80 (also named polysorbate 80), Diglycerol monocaprylate, Polyglycerol caprylate and Cremophor RH 40.
   76. An oral pharmaceutical composition according to any one of aspects 57-75, wherein the organic solvent is present in the amount from about 1% to about 15%.
   77. An oral pharmaceutical composition according to any one of aspects 57-76, wherein the modification groups at physiological pH are one or two organic substituents which are having a MW below 200 g per mol conjugated to the N-terminal of the parent insulin.
   78. An oral pharmaceutical composition according to any one of aspects 57-77, wherein modification groups at physiological pH are designated Y and Z in Formula I:

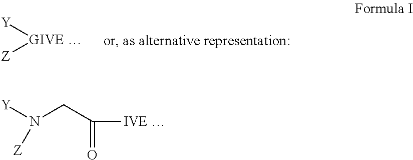

and wherein Y and Z are attached to the N-terminal amino acids of the insulin peptide.
79. An oral pharmaceutical composition according to aspect 78, wherein Y and Z are different and
Y is R—C(═X)—,
Z is H,
R is H, NH₂, straight chain or branched C1-C4 alkyl, straight chain or branched C2-C4 acyl substituted with dimethylamino, diethylamino, dipropylamino, dimethylammonium, diethylammonium or dipropylammonium, C5-C6 cycloalkyl, substituted C5-C6 cycloalkyl, 5- or 6 membered saturated heterocyclyl, substituted 5- or 6 membered saturated heterocyclyl, and
X is O or S.
   80. An oral pharmaceutical composition according to aspect 78, wherein Y and Z are different and
Y is R—C(═X)—,
Z is H,
R is H, NH₂, straight chain or branched C1-C4 alkyl, C5-C6 cycloalkyl, 5- or 6 membered saturated heterocyclyl, and
X is O or S.
   81. An oral pharmaceutical composition according to aspect 78, wherein Y═Z═C1-C4.
   82. An oral pharmaceutical composition according to aspect 78, wherein Y and Z are the same and selected from the group consisting of: dimethyl, diethyl, di-n-propyl, di-sec-propyl, di-n-butyl, di-i-butyl and amidinyl.
   83. An oral pharmaceutical composition according to aspect 78, wherein Y and Z are the same and selected from dimethyl and diethyl
   84. An oral pharmaceutical composition according to aspect 78, wherein Y and Z are the same and dimethyl.
   85. An oral pharmaceutical composition according to any one of aspects 57-84, wherein the N-terminal modification is positively charged at physiological pH.
   86. An oral pharmaceutical composition according to any one of aspects 57-84, wherein the N-terminal modification is selected from the group consisting of: N,N-di-C1-4 alkyl, N-amidinyl, 4-(N,N-dimethylamino)butanoyl, 3-(1-piperidinyl)propionyl, 3-(N,N-dimethylamino)propionyl, N,N-dimethyl-Glycine and N,N,N-trimethyl Glycine.
   87. An oral pharmaceutical composition according to aspect 86, wherein the N-terminal modification is N,N-di-C1-4 alkyl.
   88. An oral pharmaceutical composition according to aspect 87, wherein the N-terminal modification is N,N-dimethyl or N,N-diethyl.
   89. An oral pharmaceutical composition according to any one of aspects 57-80, wherein the N-terminal modification group is not malonyl or succinyl.
   90. An oral pharmaceutical composition according to any one of aspects 57-80, wherein the N-terminal modification group is not malonyl.
   91. An oral pharmaceutical composition according to any one of aspects 57-80, wherein the N-terminal modification group is not succinyl.
   92. An oral pharmaceutical composition according to any one of aspects 57-80, wherein the N-terminal modification group is selected from the group consisting of: N,N-dimethyl, N,N-diethyl, carbamoyl, formyl, acetyl, propionyl, butyryl, glutaryl, and diglycolyl.
   93. An oral pharmaceutical composition according to any one of aspects 57-80, wherein the N-terminal modification is selected from the group consisting of: Carbamoyl, thiocarbamoyl, short chain acyl groups, oxalyl, glutaryl and diglycolyl.
   94. An oral pharmaceutical composition according to any one of aspects 57-80, wherein the N-terminal modification is selected from the group consisting of: Carbamoyl, thiocarbamoyl, formyl, acetyl, propionyl, butyryl, pyroglutamyl, oxalyl, glutaryl and diglycolyl.
   95. An oral pharmaceutical composition according to any one of aspects 57-80, wherein the N-terminal modification is neutral at physiological pH.
   96. An oral pharmaceutical composition according to any one of aspects 57-80, wherein the N-terminal modification is selected from the group consisting of: Carbamoyl, thiocarbamoyl, formyl, acetyl, propionyl, butyryl, and pyroglutamyl.
   97. An oral pharmaceutical composition according to any one of aspects 57-80, wherein the N-terminal modification is negatively charged at physiological pH.
   98. An oral pharmaceutical composition according to any one of aspects 57-80, wherein the N-terminal modification is selected from the group consisting of: oxalyl, glutaryl and diglycolyl.
   99. An oral pharmaceutical composition according to any one of aspects 57-98, wherein the N-terminal modified insulin consists of a peptide part, an N-terminal modification group and optionally a lipophilic substituent attached to the peptide part, wherein the peptide part is human insulin, desB30 human insulin, human insulin with less than 8 modifications or desB30 human insulin with less than 8 modifications.
   100. An oral pharmaceutical composition according to aspect 99, wherein the peptide part is human insulin with less than 8 modifications substituted in at least one position selected from the group consisting of: A8H, A14E, A14H, A14D, A21G, desA21, B1E, desB1, B3Q, B3G, B16H, B16E, B25H, B25N, B26G, B26D, B26E, B27G, B27E, B27D, desB27, B28G, B28E, B28D, desB28 and desB30.
   101. An oral pharmaceutical composition according to aspect 99, wherein the peptide part is human insulin with less than 8 modifications substituted in at least on position selected from the group consisting of: A14E, A21G, B3Q, B16H, B16E, B25H, B25N, B26G, B27G, desB27, B28G, and desB30.
   102. An oral pharmaceutical composition according to aspect 99, wherein the peptide part is human insulin with less than 8 modifications substituted in at least two positions selected from the group consisting of: A8H, A14E, A14H, A14D, A21G, desA21, B1E, desB1, B3Q, B3G, B16H, B16E, B25H, B25N, B26G, B26D, B26E, B27G, B27E, B27D, desB27, B28G, B28E, B28D, desB28 and desB30.
   103. An oral pharmaceutical composition according to aspect 99, wherein the peptide part is human insulin with less than 8 modifications substituted in at least two positions selected from the group consisting of: A14E, A21G, B3Q, B16H, B16E, B25H, B25N, B26G, B27G, desB27, B28G, and desB30.
   104. An oral pharmaceutical composition according to any one of aspects 57-104, wherein the peptide part is human insulin with less than 8 modifications, substituted such that at least one hydrophobic amino acid has been substituted with hydrophilic amino acids, and wherein said substitution is within or in close proximity to one or more protease cleavage sites of the insulin.
   105. An oral pharmaceutical composition according to any one of aspects 57-104, wherein the peptide part is selected from the group consisting of: A14E, B25H, desB30 human insulin; A14E, B25H, desB27, desB30 human insulin; A14E, B16H, B25H, desB27, desB30 human insulin; A14E, desB27, desB30 human insulin; A14E, B16E, B25H, desB27, desB30 human insulin and B25H, desB27, desB30 human insulin.
   106. An oral pharmaceutical composition according to any one of aspects 57-104, wherein the peptide part is selected from the group consisting of: A14E, A21G, B25H, desB30 human insulin; A14E, A21G, B16H, B25H, desB30 human insulin; A14E, A21G, B16E, B25H, desB30 human insulin; A14E, A21G, B25H, desB27, desB30 human insulin; A14E, A21G, B25H, desB27, desB30 human insulin; A14E, A21G, B25H, B26G, B27G, B28G, desB30 human insulin; A21G, B25H, desB30 human insulin and A21G, B25N, desB30 human insulin.
   107. An oral pharmaceutical composition according to any one of aspects 57-104, wherein the peptide part is selected from the group consisting of: A14E, A21G, B25H, desB30 human insulin; A14E, A21G, desB27, desB30 human insulin; A14E, A21G, B16H, B25H, desB30 human insulin; A14E, A21G, B16E, B25H, desB30 human insulin; A14E, A21G, B25H, desB27, desB30 human insulin; A14E, A21G, B25H, desB27, desB30 human insulin; A21G, B25H, desB30 human insulin and A21G, B25N, desB30 human insulin.
   108. An oral pharmaceutical composition according to any one of aspects 57-104, wherein the peptide part is selected from the group consisting of: A14E, B25H, desB30 human insulin; A14E, B16H, B25H, desB30 human insulin; A14E, B16E, B25H, desB30 human insulin; A14E, desB27, desB30 human insulin; A14E, B16H, desB27, desB30 human insulin; A14E, B25H, B26G, B27G, B28G, desB30 human insulin; B25H, desB30 human insulin and A14E, B25H, desB27, desB30 human insulin.
   109. An oral pharmaceutical composition according to any one of aspects 57-108, wherein the N-terminal modified insulin consists of a peptide part, an N-terminal modification group and a lipophilic substituent attached to the peptide part, wherein the lipophilic substituent is a side chain consisting of a fatty acid or a fatty diacid attached to the insulin, optionally via a linker, in an amino acid position of the peptide part.
   110. An N-terminally modified insulin according to aspect 109, wherein the peptide part comprises only one lysine residue and the lipophilic substituent is attached, optionally via a linker, to said lysine residue.
   111. An N-terminally modified insulin according to aspect 109 or 110, wherein the lipophilic substituent has the general formula

Acy-AA1ₙ-AA2ₘ-AA3ₚ- (Formula III),

wherein
n is 0 or an integer in the range from 1 to 3;
m is 0 or an integer in the range from 1 to 10;
p is 0 or an integer in the range from 1 to 10;
Acy is a fatty acid or a fatty diacid comprising from about 8 to about 24 carbon atoms;
AA1 is a neutral linear or cyclic amino acid residue;
AA2 is an acidic amino acid residue;
AA3 is a neutral, alkyleneglycol-containing amino acid residue;
   the order by which AA1, AA2 and AA3 appears in the formula can be interchanged independently; AA2 can occur several times along the formula (e.g., Acy-AA2-AA3₂-AA2-); AA2 can occur independently (=being different) several times along the formula (e.g., Acy-AA2-AA3₂-AA2-); the connections between Acy, AA1, AA2 and/or AA3 are amide (peptide) bonds which, formally, can be obtained by removal of a hydrogen atom or a hydroxyl group (water) from each of Acy, AA1, AA2 and AA3; and attachment to the peptide part can be from the C-terminal end of a AA1, AA2, or AA3 residue in the acyl moiety of the formula (III) or from one of the side chain(s) of an AA2 residue present in the moiety of formula (III).
   112. A method of producing a N-terminally modified insulin derivative according to any one of the preceding aspects.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law).

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

### EXAMPLES

The following examples are offered by way of illustration, not by limitation.

The abbreviations used herein are the following: βAla is beta-alanyl, Aoc is 8-aminooctanoic acid, tBu is tert-butyl, CV is column volumes, DCM is dichloromethane, DIC is diisopropylcarbodiimide, DIPEA=DIEA is N,N-disopropylethylamine, DMF is N,N-dimethylformamide, DMSO is dimethyl sulphoxide, EtOAc is ethyl acetate, Fmoc is 9-fluorenylmethyloxycarbonyl, γGlu is gamma L-glutamyl, HCl is hydrochloric acid, HOBt is 1-hydroxybenzotriazole, NMP is N-methylpyrrolidone, MeCN is acetonitrile, OEG is [2-(2-aminoethoxy)ethoxy]ethylcarbonyl, Su is succinimidyl-1-yl=2,5-dioxo-pyrrolidin-1-yl, OSu is succinimidyl-1-yloxy=2,5-dioxo-pyrrolidin-1-yloxy, RPC is reverse phase chromatography, RT is room temperature, TFA is trifluoroacetic acid, THF is tetrahydrofuran, TNBS is 2,4,6-trinitrobenzenesulfonic acid, TRIS is tris(hydroxymethyl)aminomethane and TSTU is O—(N-succinimidyl)-1,1,3,3-tetramethyluronium tetrafluoroborate.

The following examples and general procedures refer to intermediate compounds and final products identified in the specification and in the synthesis schemes. The preparation of the compounds of the present invention is described in detail using the following examples, but the chemical reactions described are disclosed in terms of their general applicability to the preparation of compounds of the invention. Occasionally, the reaction may not be applicable as described to each compound included within the disclosed scope of the invention. The compounds for which this occurs will be readily recognised by those skilled in the art. In these cases the reactions can be successfully performed by conventional modifications known to those skilled in the art, that is, by appropriate protection of interfering groups, by changing to other conventional reagents, or by routine modification of reaction conditions. Alternatively, other reactions disclosed herein or otherwise conventional will be applicable to the preparation of the corresponding compounds of the invention. In all preparative methods, all starting materials are known or may easily be prepared from known starting materials. All temperatures are set forth in degrees Celsius and unless otherwise indicated, all parts and percentages are by weight when referring to yields and all parts are by volume when referring to solvents and eluents.

The compounds of the invention can be purified by employing one or more of the following procedures which are typical within the art. These procedures can—if needed—be modified with regard to gradients, pH, salts, concentrations, flow, columns and so forth. Depending on factors such as impurity profile, solubility of the insulins in question etcetera, these modifications can readily be recognised and made by a person skilled in the art.

After acidic HPLC or desalting, the compounds are isolated by lyophilisation of the pure fractions.

After neutral HPLC or anion exchange chromatography, the compounds are de-salted, precipitated at isoelectrical pH, or purified by acidic HPLC.

Typical Purification Procedures:

The HPLC system is a Gilson system consisting of the following: Model 215 Liquid handler, Model 322-H2 Pump and a Model 155 UV Dector. Detection is typically at 210 nm and 280 nm.

The Äkta Purifier FPLC system (GE Health Care) consists of the following: Model P-900 Pump, Model UV-900 UV detector, Model pH/C-900 pH and conductivity detector, Model Frac-950 Fraction collector. UV detection is typically at 214 nm, 254 nm and 276 nm. The Äkta Explorer Air FPLC system (Amersham BioGE Health Caresciences) consists of the following: Model P-900 Pump, Model UV-900 UV detector, Model pH/C-900 pH and conductivity detector, Model Frac-950 Fraction collector. UV detection is typically at 214 nm, 254 nm and 276 nm

### Acidic HPLC:

Column: Phenomenex, Gemini, 5μ, C18, 110 Å, 250×30 cm
Flow: 20 ml/min′
Eluent: A: 0.1% TFA in water B: 0.1% TFA in CH₃CN
Gradient:
   0-7.5 min: 10% B
   7.5-87.5 min: 10% B to 60% B
   87.5-92.5 min: 60% B
   92.5-97.5 min: 60% B to 100% B

### Neutral HPLC:

Column: Phenomenex, Gemini, C18, 5 μm 250×30.00 mm, 110 Å
Flow: 20 ml/min
Eluent: A: 20% CH₃CN in aqueous 10 mM TRIS+15 mM (NH₄)SO₄ pH=7.3 B: 80% CH₃CN, 20% water
Gradient:
   0-7.5 min: 0% B
   7.5-52.5 min: 0% B to 60% B
   52.5-57.5 min: 60% B
   57.5-58 min: 60% B to 100% B
   58-60 min: 100% B
   60-63 min: 10% B

### Anion Exchange Chromatography:

Column: RessourceQ, 6 ml,
Flow: 6 ml/min
Buffer A: 0.09% NH₄HCO₃, 0.25% NH₄OAc, 42.5% ethanol pH 8.4
Buffer B: 0.09% NH₄HCO₃, 2.5% NH₄OAc, 42.5% ethanol pH 8.4
Gradient: 100% A to 100% B during 30 CV
Column: Source 30Q, 30×250 mm
Flow: 80 ml/min
Buffer A: 15 mM TRIS, 30 mM Ammoniumacetat i 50% Ethanol, pH 7.5 (1.25 mS/cm)
Buffer B: 15 mM TRIS, 300 mM Ammoniumacetat i 50% Ethanol pH 7.5 (7.7 mS/cm)
Gradient: 15% B to 70% B over 40 CV
Desalting:
Column: Daiso 200 Å15 um FeFgel 304, 30×250 mm
Buffer A: 20 v/v % Ethanol, 0.2% acetic acid
Buffer B: 80% v/v % Ethanol, 0.2% acetic acid
Gradient: 0-80% B over 1.5 CV
Flow: 80 ml/min
Column: HiPrep 26/10
Flow: 10 ml/min,
Gradient: 6 CV
Buffer: 10 mM NH₄HCO₃

### General Procedure for the Solid Phase Synthesis of Acylation Reagents of the General Formula (II):

Acy-AA1ₙ-AA2ₘ-AA3ₚ-Act, (II):

wherein Acy, AA1, AA2, AA3, n, m, and p are as defined above and Act is the leaving group of an active ester, such as N-hydroxysuccinimide (OSu), or 1-hydroxybenzotriazole, and

wherein carboxylic acids within the Acy and AA2 moieties of the acyl moiety are modified as tert-butyl esters.

Compounds of the general formula (II) according to the invention can be synthesised on solid support using procedures well known to skilled persons in the art of solid phase peptide synthesis. This procedure comprises attachment of a Fmoc protected amino acid to a polystyrene 2-chlorotritylchloride resin. The attachment can, e.g., be accomplished using the free N-terminally modified amino acid in the presence of a tertiary amine, like triethyl amine or N,N-diisopropylethylamine (see references below). The C-terminal end (which is attached to the resin) of this amino acid is at the end of the synthetic sequence being coupled to the insulins of the invention. After attachment of the Fmoc amino acid to the resin, the Fmoc group is deprotected using, e.g., secondary amines, like piperidine or diethyl amine, followed by coupling of another (or the same) Fmoc protected amino acid and deprotection. The synthetic sequence is terminated by coupling of mono-tert-butyl protected fatty (α, ω) diacids, like hexadecanedioic, heptadecanedioic, octadecanedioic or eicosanedioic acid mono-tert-butyl esters. Cleavage of the compounds from the resin is accomplished using diluted acid like 0.5-5% TFA/DCM (trifluoroacetic acid in dichloromethane), acetic acid (e.g., 10% in DCM, or HOAc/trifluoroethanol/DCM 1:1:8), or hecafluoroisopropanol in DCM (See, e.g., “Organic Synthesis on Solid Phase”, F. Z. Dörwald, Wiley-VCH, 2000. ISBN 3-527-29950-5, “Peptides: Chemistry and Biology”, N. Sewald & H.-D. Jakubke, Wiley-VCH, 2002, ISBN 3-527-30405-3 or “The Combinatorial Chemistry Catalog” 1999, Novabiochem AG, and references cited therein). This ensures that tert-butyl esters present in the compounds as carboxylic acid protecting groups are not deprotected. Finally, the C-terminal carboxy group (liberated from the resin) is activated, e.g., as the N-hydroxysuccinimide ester (OSu) and used either directly or after purification as coupling reagent in attachment to insulins of the invention. This procedure is described in example 9 in, WO09115469.

Alternatively, the acylation reagents of the general formula (II) above can be prepared by solution phase synthesis as described below.

Mono-tert-butyl protected fatty diacids, such as hexadecanedioic, heptadecanedioic, octadecanedioic or eicosanedioic acid mono-tert-butyl esters are activated, e.g., as OSu-esters as described below or as any other activated ester known to those skilled in the art, such as HOBt- or HOAt-esters. This active ester is coupled with one of the amino acids AA1, mono-tert-butyl protected AA2, or AA3 in a suitable solvent such as THF, DMF, NMP (or a solvent mixture) in the presence of a suitable base, such as DIPEA or triethylamine. The intermediate is isolated, e.g., by extractive procedures or by chromatographic procedures. The resulting intermediate is again subjected to activation (as described above) and to coupling with one of the amino acids AA1, mono-tert-butyl protected AA2, or AA3 as described above. This procedure is repeated until the desired protected intermediate Acy-AA1ₙ-AA2ₘ-AA3ₚ-OH is obtained. This is in turn activated to afford the acylation reagents of the general formula (II) Acy-AA1ₙ-AA2ₘ-AA3ₚ-Act. This procedure is described in example 11 in WO09115469.

The acylation reagents prepared by any of the above methods can be (tert-butyl) de-protected after activation as OSu esters. This can be done by TFA treatment of the OSu-activated tert-butyl protected acylation reagent. After acylation of any insulin, the resulting unprotected acylated protease stabilized (parent) insulin of the invention is obtained. This procedure is described in example 16 in WO09115469.

If the reagents prepared by any of the above methods are not (tert-butyl) de-protected after activation as OSu esters, acylation of any insulin affords the corresponding tert-butyl protected acylated insulin of the invention. In order to obtain the unprotected acylated insulin of the invention, the protected insulin is to be de-protected. This can be done by TFA treatment to afford the unprotected acylated (parent) insulin of the invention. This procedure is described in example 1 in WO05012347.

Methods for preparation of acylated insulins without N-terminal protection (i.e. starting materials for preparation of N-terminally modified analogues of invention (parent insulins)) can be found in WO09115469.

### General Procedure (A) for Preparation for Reductive N-Methylation of Acylated Insulins of this Invention

The acylated insulin (0.022 mmol) is dissolved in a mixture of a polar aprotic or protic solvent, such as N-methylformamide, DMF, NMP, THF or DMSO (3.8 ml) and 0.2 M citrate buffer, sodium acetate buffer or diluted acetic acid, pH 4.5. (2.2 mL, 0.44 mmol; preparation of the buffer: citric acid 0.2 M+NaOH 0.35 M) and the mixture is gently stirred. 37% Aqueous formaldehyde solution (0.063 ml, appr. 0.82 mmol)- or acetaldehyde, if N,N-diethyl derivatives are desired—is added, followed by addition of a freshly prepared solution of sodium cyanoborohydride (21 mg, 0.33 mmol) in methanol or water (0.3 mL). The mixture is gently stirred. After completion of the reaction, the mixture is carefully acidified by dropwise addition of 1N hydrochloric acid to pH 2-3. The product is isolated by preparative HPLC.

The general procedure (A) is illustrated in example 1.

### Example 1

### General procedure (A)

### A1(N^{α}, N^{α}-Dimethyl), A14E, B1(N^{α}, N^{α}-dimethyl), B25H, B29K(N^{ε}octadecanedioyl-gGlu-2xOEG), desB30 human insulin

IUPAC (OpenEye, IUPAC style) name:

N{A1},N{A1}-dimethyl,N{B1},N{B1}-dimethyl,N{Epsilon-B29}-[2-[2-[[2-[2-[2-[[(4S)-4-carboxy-4-(17-carboxyheptadecanoylamino)butanoyl]amino]ethoxy]ethoxy]acetyl]amino]-ethoxy]ethoxy]acetyl]-[GluA14,HisB25],des-ThrB30-Insulin (human)

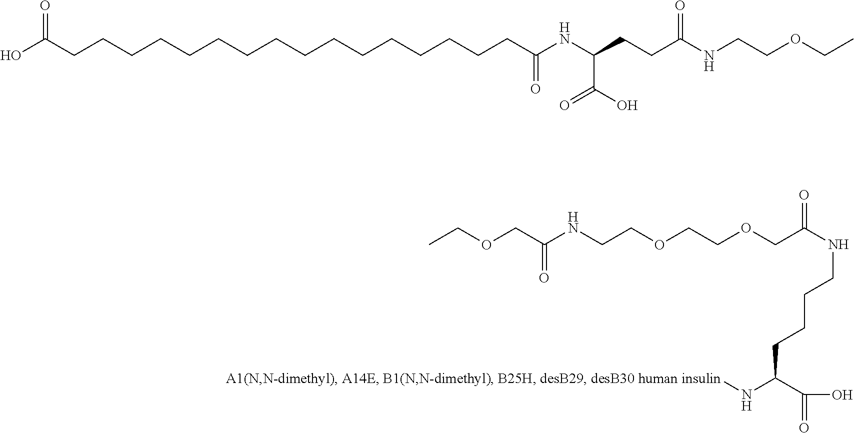

A14E, B25H, B29K(N^{ε}Octadecanedioyl-gGlu-2xOEG), desB30 human insulin (0.5 g) was dissolved in DMF (10 mL) and citrate buffer (0.2M, pH 4.5, 7 mL, prepared from 0.2 M citric acid and 0.35 M NaOH) was added. To this solution aqueous formaldehyde (37%, 0.35 mL) was added followed by sodium cyanoborohydride (80 mg) dissolved in methanol (1 mL). The resulting mixture was left at room temperature for 15 hours, and then water (10 mL) was added and pH was adjusted to 2 with 1N hydrochloric acid.

The analogue was purified by preparative HPLC:

Column: Phenomenex, Gemini, 5μ, C18, 110 Å, 250×30 cm

Flow: 20 ml/min′

Eluent: A: 0.1% TFA in water B: 0.1% TFA in CH₃CN

Gradient:
0-7.5 min: 10% B
7.5-87.5 min: 10% B to 60% B
87.5-92.5 min: 60% B
92.5-97.5 min: 60% B to 100% B
97.5-100 min: 100% B
100-103 min: 10% B

Pure fractions were pooled and lyophilized. The dry material was dissolved in water (50 mL) and added 0.1N NaOH to pH=8.1 and lyophilised to afford 0.26 g of the title insulin analogue.

MALDI-MS: m/z: 6434; calcd: 6434.

LC-MS (electrospray): (m+4)/4: 1609.65 (6434)

Similarly, the following analogues were prepared:

### Example 2

### General Procedure (A)

### A1(N^{α},N^{α}-Diethyl), A14E, B1(N^{α}, N^{α}-diethyl), B25H, B29K(N^{ε}Octadecanedioyl-gGlu-2xOEG), desB30 human insulin

IUPAC (OpenEye, IUPAC style) name:

N{A1},N{A1}-diethyl,N{B1},N{B1}-diethyl,N{Epsilon-B29}-[2-[[2-[2-[2-[[(4S)-4-carboxy-4-(17-carboxyheptadecanoylamino)butanoyl]amino]ethoxy]ethoxy]acetyl]amino]-ethoxy]ethoxy]acetyl]-[GluA14,HisB25],des-ThrB30-Insulin (human).

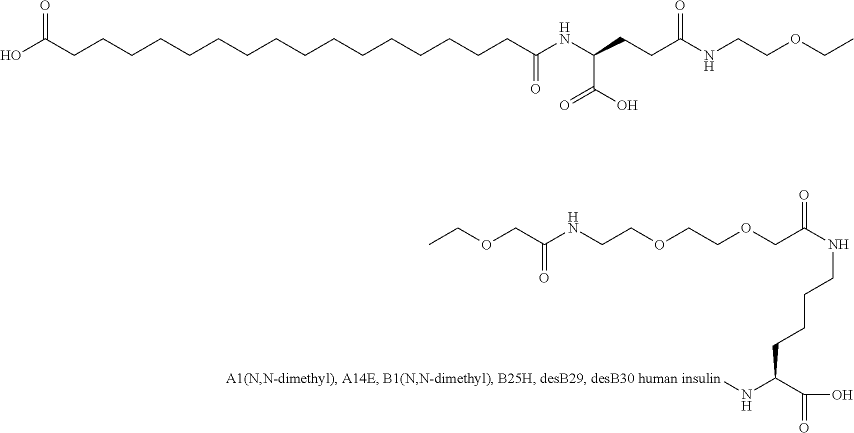

This analogue was prepared similarly as described above, but using acetaldehyde (0.43 mL). The analogue was purified first by acidic HPLC as described above, followed by neutral HPLC:

Column: Phenomenex, Gemini, 5μ, C18, 110 Å, 250×30 cm

Flow: 20 mL/min

Eluent: A: 20% CH₃CN in aqueous 10 mM TRIS+15 mM (NH₄)SO₄ pH=7.3 B: 80% CH₃CN, 20% water

Gradient:
0-7.5 min: 0% B
7.5-52.5 min: 0% B to 60% B
52.5-57.5 min: 60% B
57.5-58 min: 60% B to 100% B
58-60 min: 100% B
60-63 min: 10% B

Pure fractions were concentrated in vacuo, dissolved in water, and pH was adjusted to 2 using 1N hydrochloric acid and de-salted by HPLC:

Column: Phenomenex, Gemini, 5μ, C18, 110 Å, 250×30 cm

Flow: 20 mL/min′

Eluent: A: 0.1% TFA in water B: 0.1% TFA in CH₃CN

Gradient:
0-7.5 min: 0% B
7.5-27.5 min: 0% B to 60% B
27.5-32.5 min: 60% B
32.5-38 min: 60% B to 100% B
38-40 min: 100% B
40-43 min: 10% B

Pure fractions were pooled and lyophilized. The dry material was dissolved in water (50 mL) and added 0.1N NaOH to pH=8.1 and lyophilised to afford 0.14 g of the title insulin analogue.

MALDI-MS: m/z: 6493; calcd: 6491.

LC-MS (electrospray): (m+4)/4: 1623.6 (6490)

### Example 3

### General Procedure (A)

### A1(N^{α},N^{α}-Dimethyl), A14E, B1(N^{α}, N^{α}-dimethyl), B16H, B25H, B29K(N^{ε}hexadecanedioyl-gGlu), desB30 human insulin

IUPAC (OpenEye, IUPAC style) name:

N{A1},N{A1}-dimethyl,N{B1},N{B1}-dimethyl,N{Epsilon-B29}-[(4S)-4-carboxy-4-(15-carboxypentadecanoylamino)butanoyl]-[GluA14,HisB16,HisB25],des-ThrB30-Insulin (human).

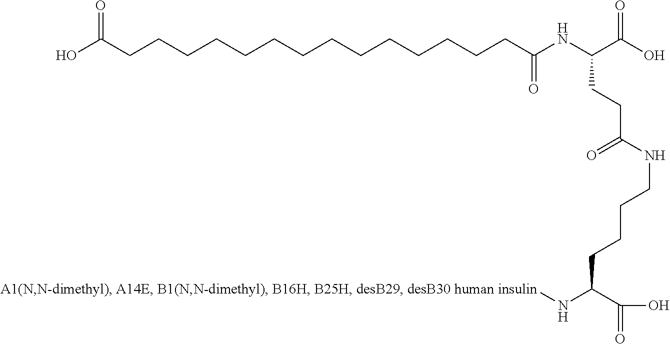

A14E, B16H, B25H, desB30 human insulin (2.2 g, protein content 49%) was dissolved in aqueous sodium carbonate (40 mL, 100 mM), and was added aqueous sodium hydroxide (1N) to pH 11. Under vigorous stirring (S)-2-(15-Carboxy-pentadecanoylamino)-pentanedioic acid 5-(2,5-dioxo-pyrrolidin-1-yl) ester (0.2 g) dissolved in N-methylpyrrolidone (NMP, 4 mL) and the resulting mixture was stirred for 5 minutes. Water (40 mL) was added and pH was adjusted to 5.7 by addition of hydrochloric acid (1N). The precipitate was isolated by centrifugation and decantation. The residue was dissolved in N,N-dimethylformamide (20 mL) and aqueous citric acid buffer (0.2 M, pH 4.5) was added. Aqueous formaldehyde (35%, 0.12 mL) and a solution of sodium cyanoborohydride (0.37 g) in methanol (8 mL) were added and the resulting mixture was allowed to stand for 6 days. Water (20 mL) and hydrochloric acid to pH 1.6 were added and the mixture was purified by HPLC. This afforded 130 mg of the title compound.

MALDI-MS: m/z: 6086; calcd: 6090.

MS (electrospray): (m+4)/4: 1523.56; calcd: 1523.53.

### Example 4

### General Procedure (A)

### A1(N^{α}, N^{α}-Dimethyl), A14E, B1(N^{α}, N^{α}-dimethyl), B25H, desB27, B29K(N^{ε}octadecanedioyl-gGlu), desB30 human insulin

IUPAC (OpenEye, IUPAC style) name:

N{A1},N{A1}-dimethyl,N{B1},N{B1}-dimethyl,N{Epsilon-B29}-[(4S)-4-carboxy-4-(17-carboxyheptadecanoylamino)butanoyl]-[GluA14,HisB25],des-ThrB27,ThrB30-Insulin (human).

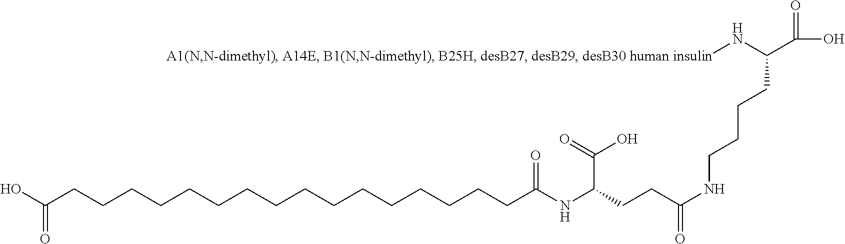

A14E, B25H, desB27, B29K(N^{ε}octadecanedioyl-gGlu), desB30 human insulin (1 g) was added DMF (10 mL) and NMP (10 mL). The resulting suspension was added citrate buffer (25 mL 0.2 M, pH 4.5). The resulting mixture (pH was 6.5) was added 1N hydrochloric acid to pH 4.5). Aqueous formaldehyde (35%, 0.18 mL) and sodium cyanoborohydride (0.2 g) were added to the mixture and the resulting mixture was stirred gently at RT for 30 min. Water (20 mL) was added to the mixture and pH was adjusted to 1.2. The mixture was purified by preparative HPLC. The pure fractions were pooled and lyophilised. The insulin was dissolved in water (70 mL) and pH was adjusted to 8.4 with 1N NaOH. Lyophilisation afforded 0.42 g of the title insulin.

MS (electrospray): (m+4)/4: 1511.69; calcd: 1511.8.

### Example 5

### General Procedure (A)

### A1(N^{α}, N^{α}-Dimethyl), A14E, B1(N^{α},N^{α}-dimethyl), B25H, desB27, B29K(N^{ε}octadecanedioyl-gGlu-2xOEG), desB30 human insulin

IUPAC (OpenEye, IUPAC style) name:

N{A1},N{A1}-dimethyl,N{B1},N{B1}-dimethyl,N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]-ethoxy]ethoxy]acetyl]-[GluA14,HisB25],des-ThrB27,ThrB30-Insulin (human).

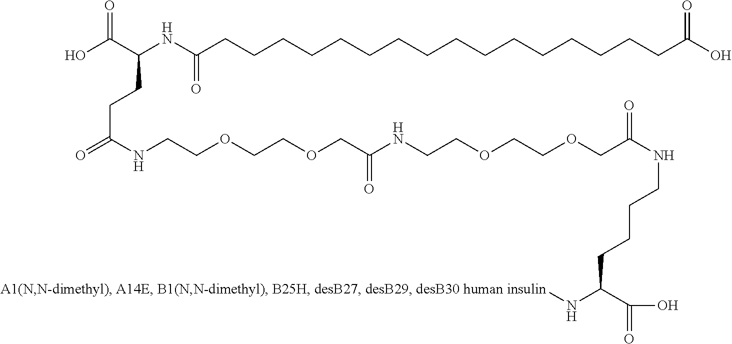

A solution of A14E, B25H, B29K(N^{ε}octadecanedioyl-gGlu-2xOEG), desB27, desB30 human insulin (600 mg) in water (15 ml) and THF (10 ml) was pH adjusted to 4.2 using glacial acetic acid. Formaldehyde (37%, 0.078 ml) was added followed by sodium cyanoborohydride (48 mg). The mixture was stirred at RT for 30 min. pH was adjusted to 11.5 with 1N NaOH. The mixture was left for 30 min before readjustment of pH to 8 with 1N NaOH. The mixture was diluted with 50% ethanol to 400 ml and 1.4 mS/cm. The mixture was purified by anion exchange as follows using Äkta Explorer Air:
Column: Source 30Q, 30×250 mm
Flow: 60 ml/min
Buffer A: 15 mM TRIS, 30 mM Ammoniumacetat i 50% Ethanol, pH 7.5 (1.25 mS/cm)
Buffer B: 15 mM TRIS, 300 mM Ammoniumacetat i 50% Ethanol pH 7.5 (7.7 mS/cm)
Gradient: 15% B to 70% B over 7 CV

The compound was collected in 400 ml and diluted to 800 ml with water before desalting:

Column: Daiso 200 Å15 um FeFgel 304, 30×250 mm

Buffer A: 20 v/v % Ethanol, 0.2% acetic acid

Buffer B: 80% v/v % Ethanol, 0.2% acetic acid

Gradient: 0-80% B over 1.5 CV

Flow: 80 ml/min

The collected compound was concentrated in vacuo to remove ethanol. pH was adjusted to 8.1 with 1N NaOH and lyophilized.

LC-MS (electrospray): (m+4)/4: 1584.15; calcd: 1584.36.

### Example 6

### General Procedure (A)

### A1(N,N-Dimethyl), A14E, B1(N,N-dimethyl), desB27, B29K(N^{ε}octadecanedioyl-gGlu-2xOEG), desB30 human insulin

IUPAC (OpenEye, IUPAC style) name:

N{A1}, N{A1}-dimethyl,N{B1},N{B1}-dimethyl, N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]-ethoxy]ethoxy]acetyl]-[GluA14],des-ThrB27,ThrB30-Insulin (human)

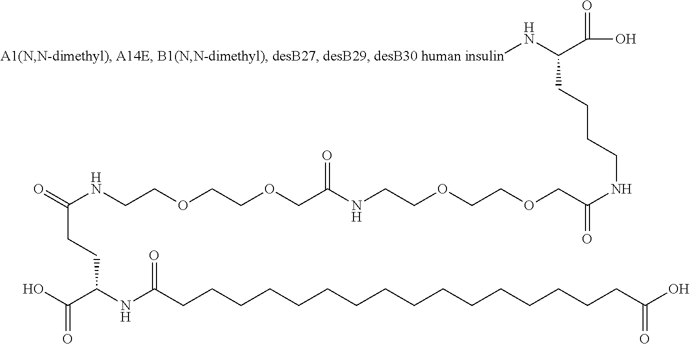

This analogue was prepared according to general procedure A.

LC-MS (electrospray): (m+4)/4: 1586.31; calcd: 1586.85.

### Example 7

### General Procedure (A)

### A1(N^{α},N^{α}-Dimethyl), A14E, B1(NP,N^{α}-dimethyl), B16H, B25H, B29K(N^{ε}eicosanedioyl-gGlu-2xOEG), desB30 human insulin

IUPAC (OpenEye, IUPAC style) name:

N{A1}, N{A1}-dimethyl, N{B1},N{B1}-dimethyl, N{Epsilon-B29}-[2-[2-[2-[[2-[2-[2-[[(4S)-4-carboxy-4-(19-carboxynonadecanoylamino)butanoyl]amino]ethoxy]ethoxy]acetyl]amino]-ethoxy]ethoxy]acetyl]-[GluA14,HisB16,HisB25],des-ThrB30-Insulin (human)

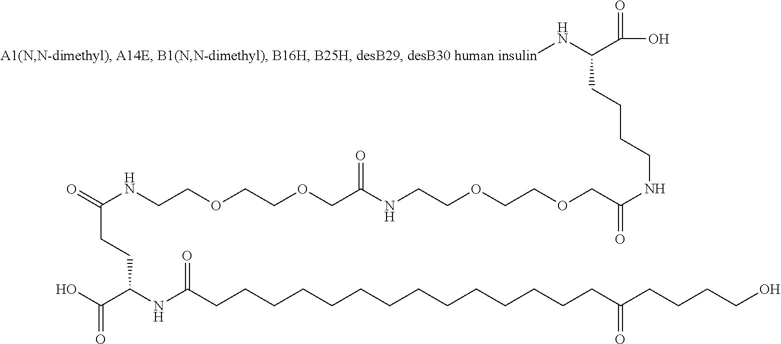

This analogue was prepared similarly as described above, using formaldehyde. The analogue was purified by acidic HPLC as described above:

LC-MS (electrospray): (m+4)/4: 1610 calcd: 1610.1.

### Example 8

### General Procedure (A)

### A1G(N^{α},N^{α}-Dimethyl), A14E, B1F(N^{α},N^{α}-dimethyl), B25H, desB27, B29K(N^{ε}hexadecanedioyl-gGlu), desB30 human insulin

IUPAC (OpenEye, IUPAC style) name:

N{A1},N{A1}-dimethyl,N{B1},N{B1}-dimethyl,N{Epsilon-B29}-[(4S)-4-carboxy-4-(15-carboxypentadecanoylamino)butanoyl]-[GluA14,HisB25],des-ThrB27,ThrB30-Insulin (human)

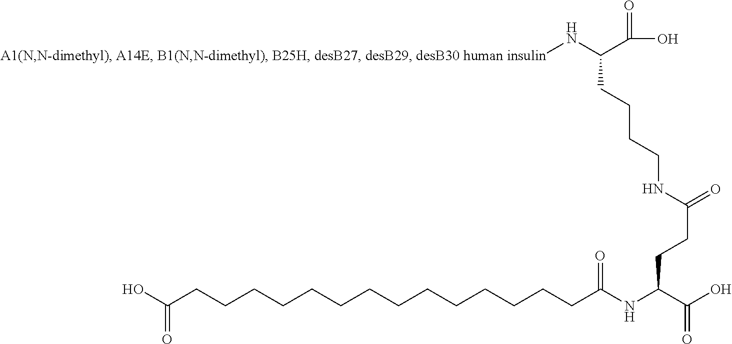

LC-MS (electrospray): m/z=1505 (M+1)/4; calcd: 1505.

### Example 9

### General Procedure (A)

### A1G(N^{α}, N^{α}-Dimethyl), A14E, B1F(N(alpha), N(N^{α}, N^{α}-dimethyl), B25H, desB27, B29K(N^{ε}hexadecanedioyl-gGlu-2xOEG), desB30 human insulin

IUPAC (OpenEye, IUPAC style) name:

N{A1},N{A1}-dimethyl,N{B1},N{B1}-dimethyl,N{Epsilon-B29}-[2-[2-[2-[[2-[2-[2-[[(4S)-4-carboxy-4-(15-carboxypentadecanoylamino)butanoyl]amino]ethoxy]ethoxy]acetyl]amino]-ethoxy]ethoxy]acetyl]-[GluA 14,HisB25],des-ThrB27,ThrB30-Insulin (human)

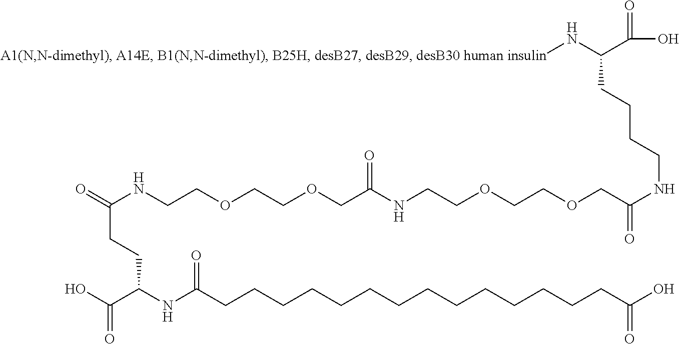

LC-MS (electrospray): m/z=1577 (M+1)/4; calcd: 1577

The analogues in the following examples may be prepared similarly:

### Example 10

### General Procedure (A)

### A1(N^{α},N^{α}-Dimethyl), A14E, B1(N^{α},N^{α}-dimethyl), desB27, B29K(N^{ε}octadecanedioyl-gGlu), desB30 human insulin

IUPAC (OpenEye, IUPAC style) name:

N{A1},N{A1}-dimethyl, N{B1},N{B1}-dimethyl, N{Epsilon-B29}-[(4S)-4-carboxy-4-(17-carboxyheptadecanoylamino)butanoyl]-[GluA14],des-ThrB27,ThrB30-Insulin (human)

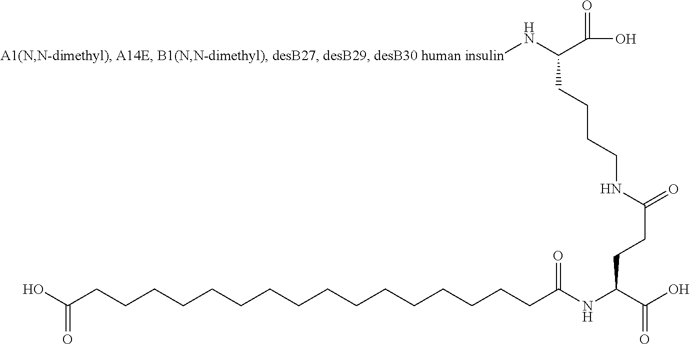

### Example 11

### General Procedure (A)

A1(N^{α}, N^{α}-Dimethyl), A14E, B1(N^{α},N^{α}-dimethyl), B25H, B29K(N^{ε}octadecanedioyl-gGlu), desB30 human insulin

IUPAC (OpenEye, IUPAC style) name:

N{A1},N{A1}-dimethyl, N{B1},N{B1}-dimethyl,N{Epsilon-B29}-[(4S)-4-carboxy-4-(17-carboxyheptadecanoylamino)butanoyl]-[GluA14,HisB25],des-ThrB30-Insulin (human)

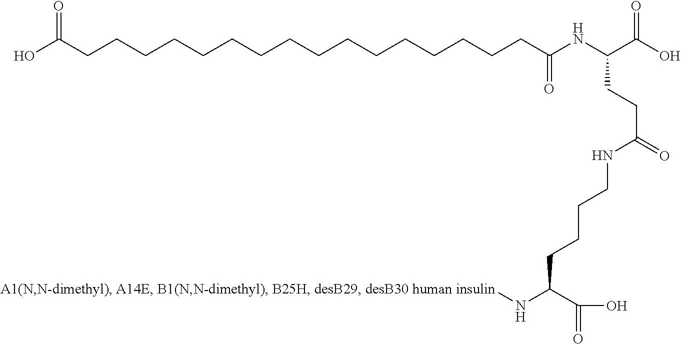

### General Procedure (B) for Preparation for Carbamoylation of Acylated Insulins of this Invention

The acylated insulin is dissolved in a buffer around physiological pH and an excess of sodium or potassium cyanate is added. The mixture is allowed to stand to completion of the reaction. If necessary, more cyanate is added. The product is isolated by preparative HPLC ion exchange chromatography, or desalting.

The general procedure (B) is illustrated in the following example.

### Example 12

### General Procedure (B)

### A1(N^{α}Carbamoyl), A14E, B1(N^{α}Carbamoyl), B25H, B29K(N^{ε}octadecanedioyl-gGlu-2xOEG), desB30 human insulin

IUPAC (OpenEye, IUPAC style) name:

N{A1}-carbamoyl, N{B1}-carbamoyl, N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]ethoxy]-ethoxy]acetyl]-[GluA14,HisB25],des-ThrB30-Insulin (human).

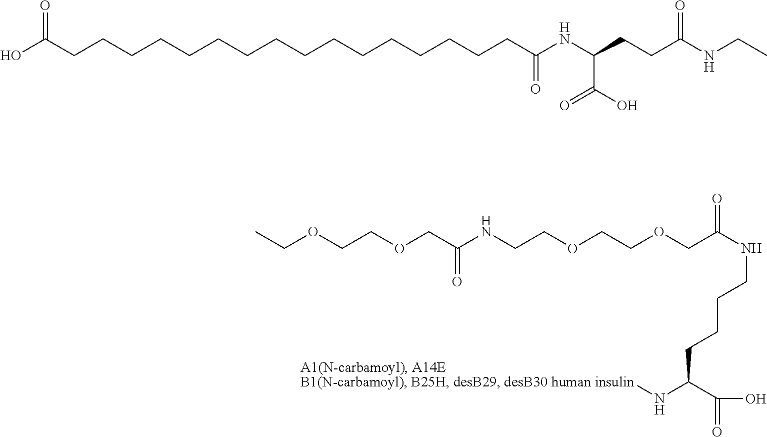

A14E, B25H, B29K(MOctadecanedioyl-gGlu-OEG-OEG), desB30 human insulin (0.4 g) was dissolved in sodium phosphate buffer (0.1M, pH 7.3, 40 mL) and potassium cyanate (300 mg) was added. The mixture was left at room temperature for 3 days. Optionally, more potassium cyanate is added during the reaction. Hydrochloric acid (0.1N) was added to pH 1.6 and the analogue was purified by preparative HPLC:

Column: Phenomenex, Gemini, 5μ, C18, 110 Å, 250×30 cm

Flow: 20 mL/min′

Eluent: A: 0.1% TFA in water B: 0.1% TFA in CH₃CN

Gradient:
0-7.5 min: 0% B
7.5-22.5 min: 0% B to 60% B
22.5-27.5 min: 60% B
27.5-33 min: 60% B to 100% B
33-38 min: 100% B

Pure fractions were pooled and lyophilised. Water was added, and pH was adjusted to 8.1 with 0.1N NaOH, and the mixture was lyophilised to afford 0.172 g of the title insulin.

MALDI-MS: m/z: 6465; calcd: 6464.

LC-MS (electrospray): (m+4)/4: 1616.9, calcd: 1617.2.

### Example 13

### General Procedure (B)

### A1(N^{α}Carbamoyl), A14E, B1(N^{α}Carbamoyl), B25H, B29K(N^{ε}hexadecanedioyl-gGlu), desB30 human insulin

IUPAC (OpenEye, IUPAC style) name:

N{A1}-carbamoyl,N{B1}-carbamoyl, N{Epsilon-B29}-[(4S)-4-carboxy-4-(15-carboxypentadecanoylamino)butanoyl]-[GluA14,HisB25],des-ThrB30-Insulin (human).

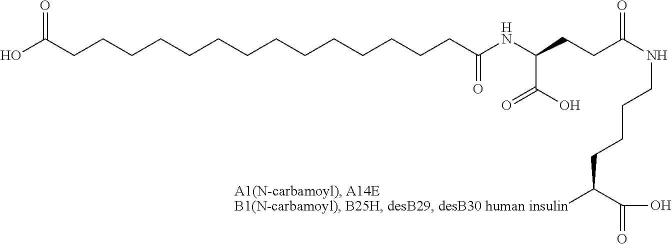

LC-MS (electrospray): (m+4)/4: 1538; calcd: 1538.

### Example 14

### General Procedure (B)

### A1(N^{α}Carbamoyl), A14E, B1(N^{α}Carbamoyl), B25H, B29K(Feicosanedioyl-gGlu), desB30 human insulin

IUPAC (OpenEye, IUPAC style) name:

N{A1}-carbamoyl,N{B1}-carbamoyl,N{Epsilon-B29}-[(4S)-4-carboxy-4-(19-carboxynonadecanoylamino)butanoyl]-[GluA14,HisB25],des-ThrB30-Insulin (human).

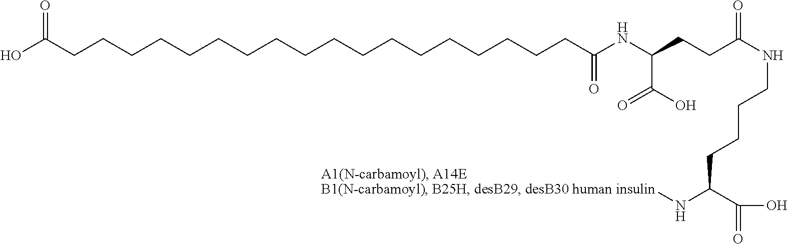

This analogue was prepared similarly as described above. The analogue was purified by acidic HPLC as described above in Example 10

MALDI-MS: m/z: 6202.75; calcd: 6202.16.

LC-MS (electrospray): (m+4)/4: 1551.29; calcd: 1551.55.

### Example 15

### General Procedure (B)

### A1(N^{α}Carbamoyl), A14E, B1(N^{α}Carbamoyl), B25H, B29K(N^{ε}eicosanedioyl-gGlu-2xOEG), desB30 human insulin

IUPAC (OpenEye, IUPAC style) name:

N{A1}-carbamoyl,N{B1}-carbamoyl,N{Epsilon-B29}-[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]acetyl]-[GluA14,HisB25],des-ThrB30-Insulin (human).

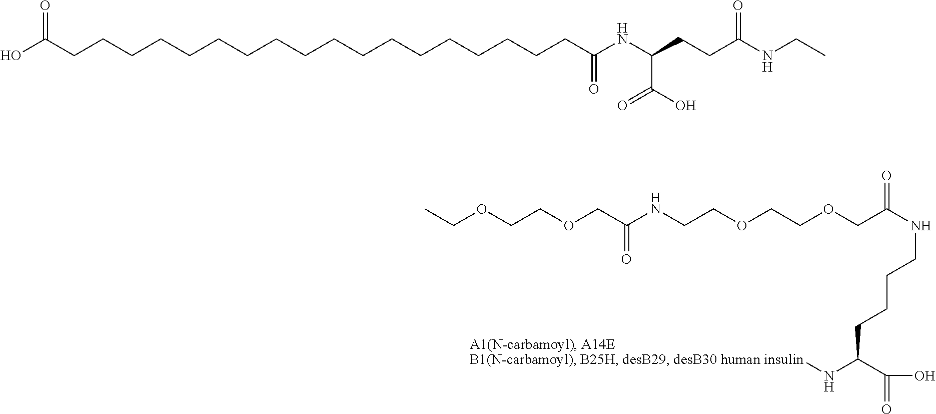

This analogue was prepared similarly as described above. The analogue was purified by acidic HPLC as described above in Example 10

MALDI-MS: m/z: 6493.52; calcd: 6491.84.

LC-MS (electrospray): (m+4)/4: 1623.96; calcd: 1624.1.

### Example 16

### General Procedure (B)

### A1(N^{α}-Carbamoyl), A14E, B1(N^{α}-Carbamoyl), B16H, B25H, B29K(N^{ε}eicosanedioyl-gGlu-2xOEG), desB30 human insulin

IUPAC (OpenEye, IUPAC style) name:

N{A1}-carbamoyl, N{B1}-carbamoyl,NN{Epsilon-B29}-[2-[2-[2-[[2-[2-[2-[[(4S)-4-carboxy-4-(19-carboxynonadecanoylamino)butanoyl]amino]ethoxy]ethoxy]acetyl]amino]ethoxy]-ethoxy]acetyl]-[GluA14,HisB16,HisB25],des-ThrB30-Insulin (human).

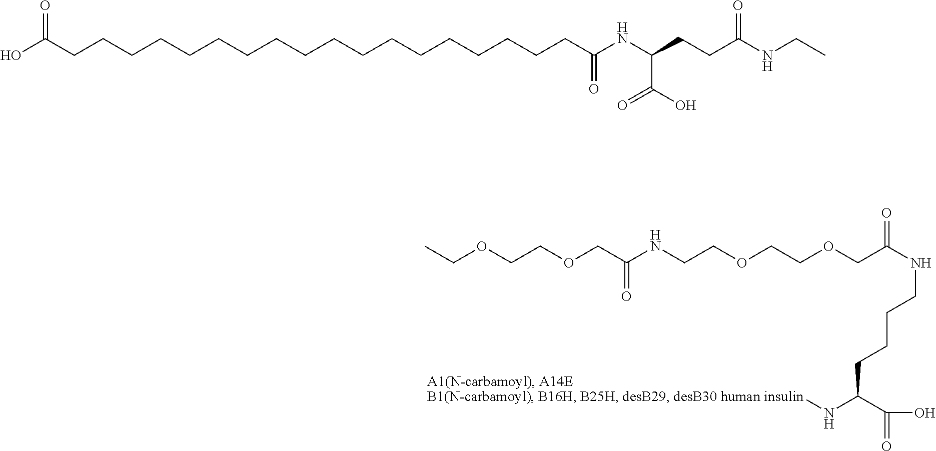

This analogue was prepared similarly as described above. The analogue was purified by acidic HPLC as described above in Example 10

MALDI-MS: m/z: 6469.46; calcd: 6466.45.

LC-MS (electrospray): (m+4)/4: 1617.4; calcd: 1617.6.

### Example 17

### General Procedure (B)

### A1(N^{α}Carbamoyl), A14E, B1(N^{α}Carbamoyl), B25H, desB27, B29K(N^{ε}octadecandioyl-gGlu), desB30 human insulin

IUPAC (OpenEye, IUPAC style) name:

N{A1}-carbamoyl, N{B1}-carbamoyl, N{Epsilon-B29}-[(4S)-4-carboxy-4-(17-carboxyheptadecanoylamino)butanoyl]-[GluA14,HisB25],des-ThrB27,ThrB30-Insulin (human).

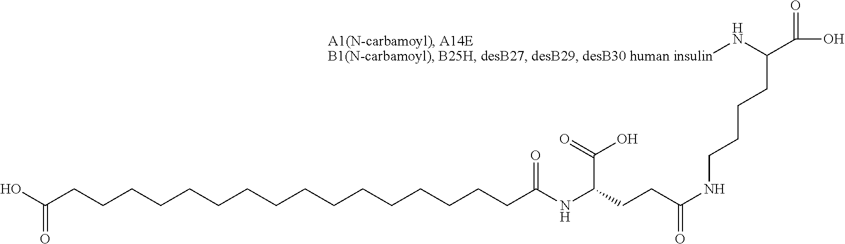

A14E, B25H, desB27, B29K B29K(N^{ε}octadecandioyl-gGlu), desB30 human insulin (1 g) was dissolved in sodium phosphate buffer (pH 7.3, 50 mL). Potassium cyanate (1.01 g) in water (10 mL) was added in 5 portions over 5 h, More potassium cyanate (200 mg) was added and the mixture stirred gently overnight. The mixture was subsequently purified by preparative HPLC. The pure fractions were pooled, lyophilised and then dissolved in water and the pH was adjusted to 7.8 with 1N NaOH. Lyophilisation afforded 359 mg of the title insulin.

LC-MS (electrospray): (m+4)/4: 1519.38; calcd: 1519.3.

### Example 18

### General Procedure (B)

### A1(N^{α}Carbamoyl), A14E, B1(N^{α}Carbamoyl), B25H, desB27, B29K(N^{ε}octadecandioyl-gGlu-2xOEG), desB30 human insulin

IUPAC (OpenEye, IUPAC style) name:

N{A1}-carbamoyl,N{B1}-carbamoyl,N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]ethoxy]-ethoxy]acetyl]-[GluA14,HisB25],des-ThrB27,ThrB30-Insulin (human).

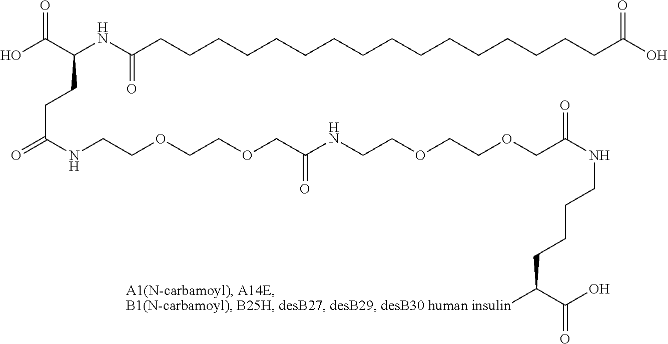

A14E, B25H, desB27, B29K B29K(N^{ε}octadecandioyl-gGlu), desB30 human insulin (1 g) was treated with potassium cyanate (0.8 g) exactly as described above.

Yield of title insulin: 210 mg.

LC-MS (electrospray): (m+4)/4: 1591.84; calcd: 1591.8.

### Example 19

### General Procedure (B)

### A1G(N(alpha)carbamoyl), A14E, B1F(N(alpha)carbamoyl), desB27, B29K(N(eps)hexadecanedioyl-gGlu), desB30 human insulin

IUPAC (OpenEye, IUPAC style) name:

N{A1}-carbamoyl,N{B1}-carbamoyl,N{Epsilon-B29}-[(4S)-4-carboxy-4-(15-carboxypentadecanoylamino)butanoyl]-[GluA14],des-ThrB27,ThrB30-Insulin (human)

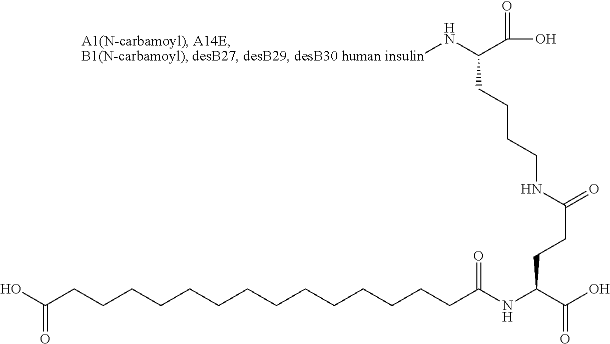

LC-MS (electrospray): m/z=1515 (M+1)/4; calcd: 1515.

### Example 20

### General Procedure (B)

### A1G(N(alpha)carbamoyl), A14E, B1F(N(alpha)carbamoyl), desB27, B29K(Neps)hexadecanedioyl-gGlu-2xOEG), desB30 human insulin

IUPAC (OpenEye, IUPAC style) name:

N{A1}-carbamoyl, N{B1}-carbamoyl,N{Epsilon-B29}-[2-[2-[2-[[2-[2-[2-[[(4S)-4-carboxy-4-(15-carboxypentadecanoylamino)butanoyl]amino]ethoxy]ethoxy]acetyl]amino]ethoxy]ethoxy]-acetyl]-[GluA14],des-ThrB27,ThrB30-Insulin (human)

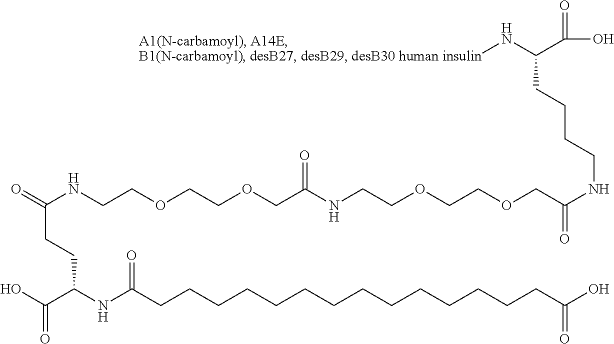

LC-MS (electrospray): m/z=1588 (M+1)/4; calcd: 1588.

### Example 21

### General Procedure (B)

### A1G(N(alpha)carbamoyl), A14E, B1F(N(alpha)carbamoyl), desB27, B29K(Neps)-eicosanedioyl-gGlu), desB30 human insulin

IUPAC (OpenEye, IUPAC style) name:

N{A1}-carbamoyl,N{B1}-carbamoyl, N{Epsilon-B29}-[(4S)-4-carboxy-4-(19-carboxynonadecanoylamino)butanoyl]-[GluA14],des-ThrB27,ThrB30-Insulin (human)

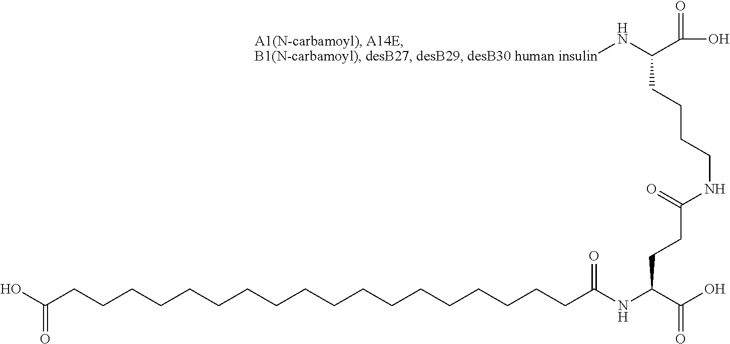

LC-MS (electrospray): m/z=1529 (M+1)/4; calcd: 1529.

### Example 22

### General Procedure (B)

### A1G(N^{α}carbamoyl), A14E, B1F(N^{α}carbamoyl), B16H, desB27, B29K(Neps)-eicosanedioyl-gGlu-2xOEG), desB30 human insulin

IUPAC (OpenEye, IUPAC style) name:

N{A1}-carbamoyl, N{B1}-carbamoyl, N{Epsilon-B29}-[2-[2-[2-[[2-[2-[2-[[(4S)-4-carboxy-4-(19-carboxynonadecanoylamino)butanoyl]amino]ethoxy]ethoxy]acetyl]amino]-ethoxy]ethoxy]acetyl]-[GluA14,HisB16,HisB25],des-ThrB27,ThrB30-Insulin (human)

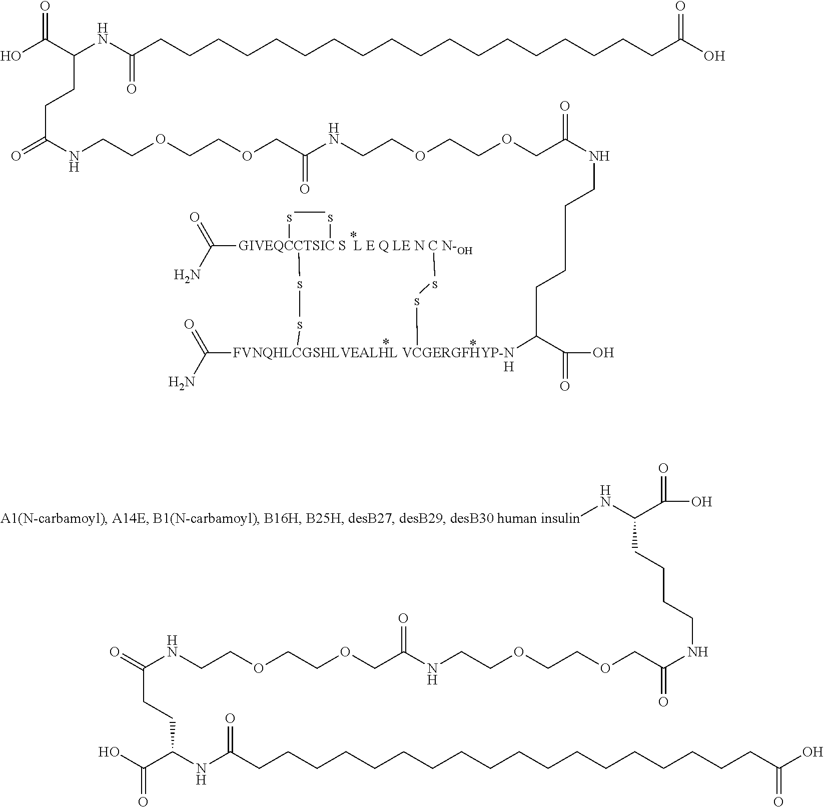

LC-MS (electrospray): m/z=1592.37 (M+1)/4; calcd: 1592.33.

### Example 23

### General Procedure (B)

### A1(N^{α}Carbamoyl), A14E, B1(N^{α}Carbamoyl), desB27, B29K(N^{ε}octadecanedioyl-gGlu), desB30 human insulin

IUPAC (OpenEye, IUPAC style) name:

N{A1}-carbamoyl,N{B1}-carbamoyl, N{Epsilon-B29}-[(4S)-4-carboxy-4-(17-carboxyheptadecanoylamino)butanoyl]-[GluA14],des-ThrB27,ThrB30-Insulin (human)

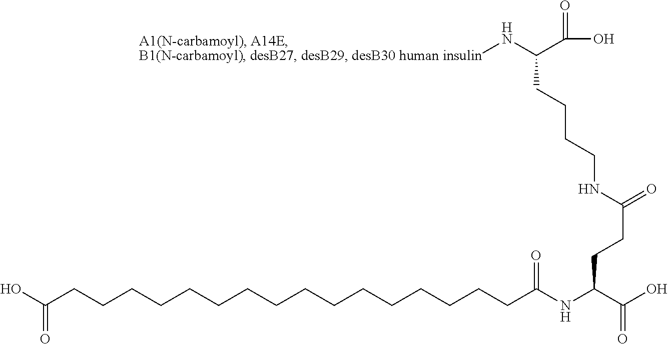

LC-MS (electrospray): m/z=1522.3 (M+1)/4; calcd: 1521.8.

The insulins in the following examples may be prepared similarly:

### Example 24

### General Procedure (B)

### A1(N^{α}Carbamoyl), A14E, B1(N^{α}Carbamoyl), B16H, B25H, B29K(N^{ε}eicosanedioyl-gGlu), desB30 human insulin

IUPAC (OpenEye, IUPAC style) name:

N{A1}-carbamoyl, N{B1}-carbamoyl, N{Epsilon-B29}-[(4S)-4-carboxy-4-(19-carboxynonadecanoylamino)butanoyl]-[GluA14,HisB16, HisB25],des-ThrB30-Insulin (human)

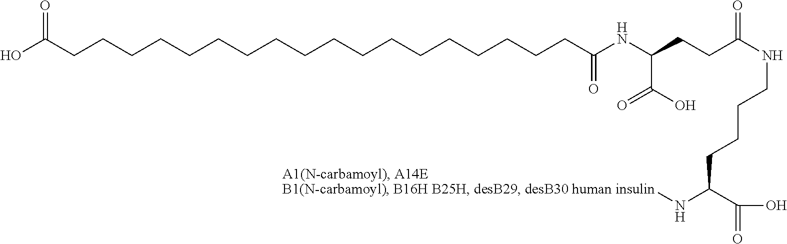

The insulin in the following example was prepared similarly:

### Example 25

### General Procedure (B)

### A1(N^{α}Carbamoyl), A14E, B1(N^{α}Carbamoyl), desB27, B29K(N^{ε}octadecanedioyl-gGlu-2xOEG), desB30 human insulin

IUPAC (OpenEye, IUPAC style) name:

N{A1}-carbamoyl, N{B1}-carbamoyl,N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]-ethoxy]ethoxy]acetyl]-[GluA14],des-ThrB27,ThrB30-Insulin (human)

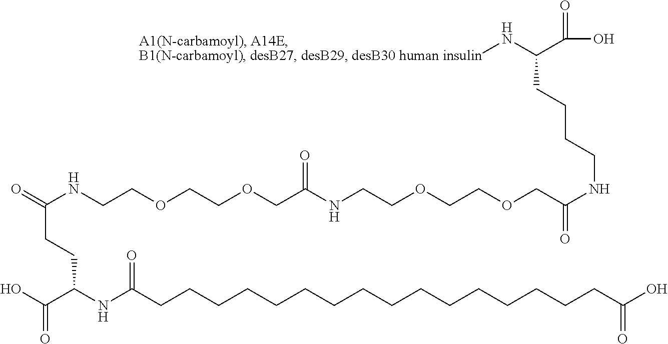

LC-MS (electrospray): m/z=1594.3 (M+1)/4; calcd: 1594.4.

The following insulins may be prepared similarly.

### Example 26

### General Procedure (B)

### A1(N^{α}Carbamoyl), A14E, B1(N^{α}carbamoyl), B25H, B29K(N^{ε}octadecanedioyl-gGlu), desB30 human insulin

IUPAC (OpenEye, IUPAC style) name:

N{A1}-carbamoyl,N{B1}-carbamoyl, N{Epsilon-B29}-[(4S)-4-carboxy-4-(17-carboxyheptadecanoylamino)butanoyl]-[GluA14,HisB25],des-ThrB30-Insulin (human)

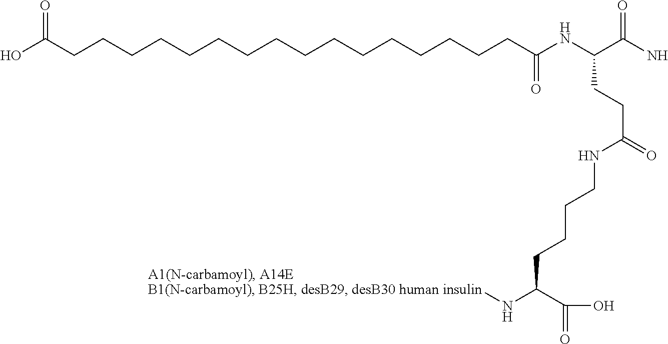

### Example 27

### General Procedure (B)

### A1(N^{α}Carbamoyl), A14E, B1(N^{α}-Carbamoyl), B16H, B25H, B29K(N^{ε}eicosanedioyl-gGlu), desB30 human insulin

IUPAC (OpenEye, IUPAC style) name:

N{A1}-carbamoyl,N{B1}-carbamoyl, N{Epsilon-B29}-[(4S)-4-carboxy-4-(19-carboxynonadecanoylamino)butanoyl]-[GluA14,HisB16,HisB25],des-ThrB30-Insulin (human)

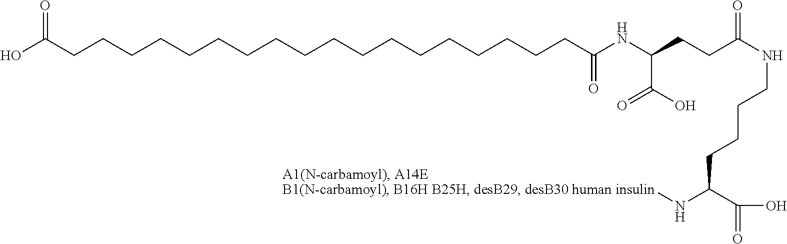

The following analogues were prepared similarly as described above.

### Example 28

### General Procedure (B)

### A1G(N^{α}carbamoyl), A14E, B1F(N^{α}carbamoyl), B25H, desB27, B29K(N^{ε}eicosanedioyl-gGlu-2xOEG), desB30 human insulin

IUPAC (OpenEye, IUPAC style) name:

N{A1}-carbamoyl, N{B1}-carbamoyl, N{Epsilon-B29}-[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]-acetyl]-[GluA14,HisB25],des-ThrB27,ThrB30-Insulin (human)

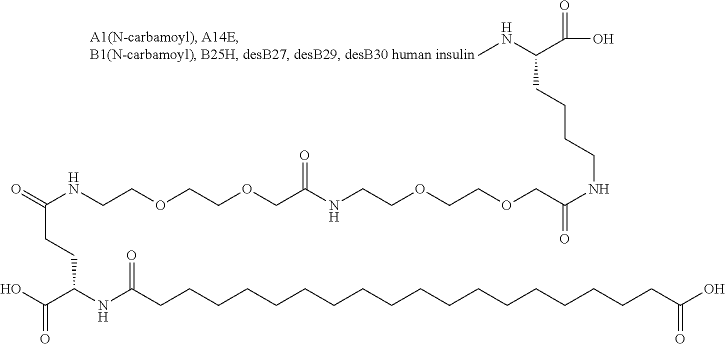 [SMILES: CN[C@@H](CCCCNC(=O)COCCOCCNC(=O)COCCOCCNC(=O)CC[C@H](NC(=O)CCCCCCCCCCCCCCCCCCC(=O)O)C(=O)O)C(=O)O]

LC-MS (electrospray): m/z=1599.0 (M+1)/4; calcd: 1598.9.

### Example 29

### General Procedure (B)

### A1G(N^{α}carbamoyl), A14E, B1F(N^{α}carbamoyl), desB27, B29K(N^{ε}eicosanedioyl-gGlu-2xOEG), desB30 human insulin

IUPAC (OpenEye, IUPAC style) name:

N{A1}-carbamoyl, N{B1}-carbamoyl, N{Epsilon-B29}-[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]-acetyl]-[GluA14],des-ThrB27,ThrB30-Insulin (human)

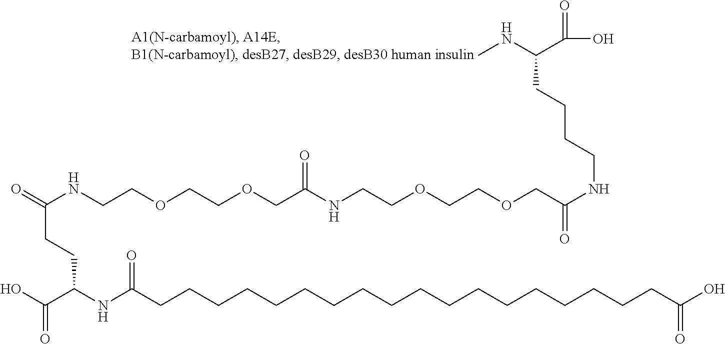 [SMILES: CN[C@@H](CCCCNC(=O)COCCOCCNC(=O)COCCOCCNC(=O)CC[C@H](NC(=O)CCCCCCCCCCCCCCCCCCC(=O)O)C(=O)O)C(=O)O]

LC-MS (electrospray): m/z=1601.7 (M+1)/4; calcd: 1601.4.

### Example 30

### General Procedure (B)

### A1G(N^{α}carbamoyl), A14E, B1F(N^{α}carbamoyl), B16H, desB27, B29K(N^{ε}eicosanedioyl-gGlu-2xOEG), desB30 human insulin

IUPAC (OpenEye, IUPAC style) name:

N{A1}-carbamoyl,N{B1}-carbamoyl, N{Epsilon-B29}-[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]-acetyl]-[GluA14,HisB16],des-ThrB27,ThrB30-Insulin (human)

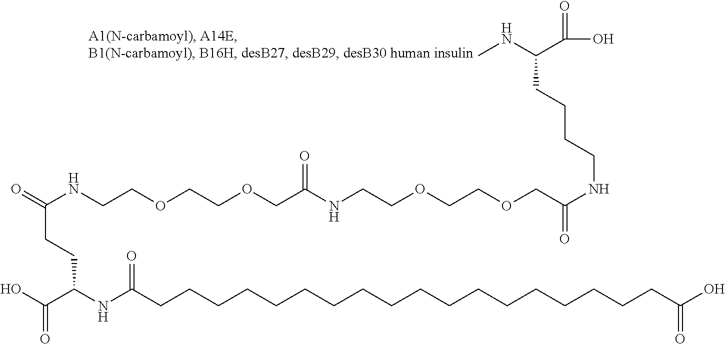 [SMILES: CN[C@@H](CCCCNC(=O)COCCOCCNC(=O)COCCOCCNC(=O)CC[C@H](NC(=O)CCCCCCCCCCCCCCCCCCC(=O)O)C(=O)O)C(=O)O]

LC-MS (electrospray): m/z=1594.98 (M+1)/4; calcd: 1594.85.

The following insulins may be prepared similarly

### Example 31

### General Procedure (B)

### A1G(N^{α}thiocarbamoyl), A14E, B1F(NN^{α}thiocarbamoyl), B25H, desB27, B29K(N^{ε}-octadecanedioyl-gGlu-2xOEG), desB30 human insulin

IUPAC (OpenEye, IUPAC style) name:

N{A1}-carbamothioyl,N{B1}-carbamothioyl,N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]ethoxy]-ethoxy]acetyl]-[GluA14,HisB25],des-ThrB27,ThrB30-Insulin (human)

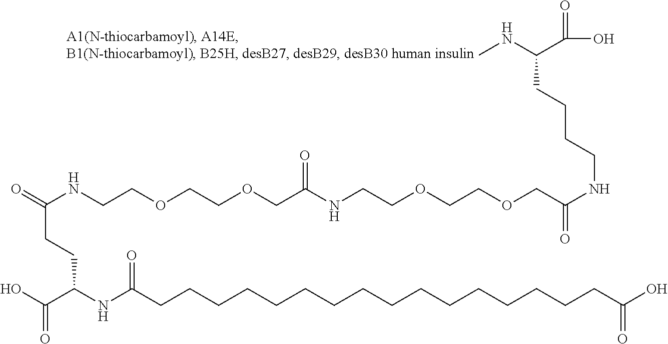 [SMILES: CN[C@@H](CCCCNC(=O)COCCOCCNC(=O)COCCOCCNC(=O)CC[C@H](NC(=O)CCCCCCCCCCCCCCCCC(=O)O)C(=O)O)C(=O)O]

This analogue may be prepared similarly as described above for the carbamoyl derivatives, using potassium thiocyanate instead of potassium cyanate.

### General Procedure (C) for Preparation for N-Terminal Acylation of Acylated Insulins of this Invention

The lysine-acylated insulin is dissolved in a buffer, optionally containing an organic co-solvent. pH of the mixture may be from neutral to alkaline (e.g. from around 6-8—depending on the solubility of the insulin in question—up to 13 or 14) and an excess of acylation reagent, eg. as N-hydroxysuccinimide ester (OSu), is added. The mixture is allowed to stand to completion of the reaction. If necessary, more acylation reagent is added. The product is isolated by preparative HPLC.

Alternatively, the reaction may be performed under anhydrous conditions, eg in DMSO containing an organic base, e.g. triethylamine.

The general procedure (C) is illustrated in the following example.

### Example 32

### General Procedure (C)

### A1(N^{α}Acetyl), A14E, B1(N^{α}Acetyl), B25H, B29K(N^{ε}hexadecanedioyl-gGlu), desB30 human insulin

IUPAC (OpenEye, IUPAC style) name:

N{A1}-acetyl,N{B1}-acetyl, N{Epsilon-B29}-[(4S)-4-carboxy-4-(15-carboxypentadecanoylamino)butanoyl]-[GluA14,HisB25],des-ThrB30-Insulin (human).

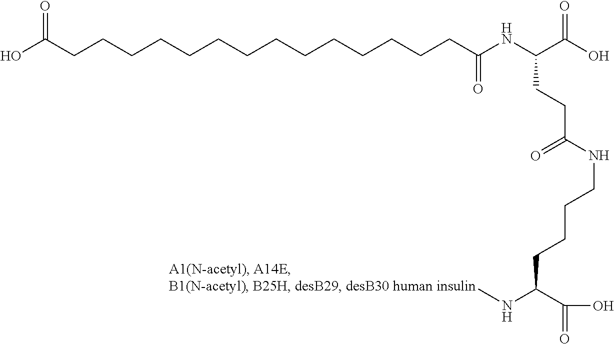 [SMILES: CN[C@@H](CCCCNC(=O)CC[C@H](NC(=O)CCCCCCCCCCCCCCC(=O)O)C(=O)O)C(=O)O]

A14E, B25H, B29K(N hexadecanedioyl-gGlu), desB30 human insulin (0.4 g, 0.066 mmol) was dissolved in a 1:1 mixture of ethanol and 0.1 M aqueous Na₂CO₃ (10 mL) and pH was adjusted to 7.4 with 1N hydrochloric acid. Acetic acid N-hydroxysuccinimide ester (60 mg, 0.38 mmol) dissolved in N,N-dimethyl formamide (2 mL) was quickly added dropwise. The mixture was allowed to stand for 3 hours, and pH rose to 9. A few drops aqueous methylamine was added and the mixture was lyophilised. The dry material was dissolved in acetic acid glacial, ethanol and water (10, 5 and 20 mL, respectively) and purified by HPLC. Pure fractions were pooled and lyophilised. This afforded 245 mg (60%) of the title insulin.

LC-MS (electrospray): (m+4)7/4: 1537; calcd: 1537.

### Example 33

### General Procedure (C)

### A1(N^{α}Acetyl), A14E, B1(N^{α}Acetyl), B25H, desB27, B29K(N^{ε}octadecanedioyl-gGlu), desB30 human insulin

IUPAC (OpenEye, IUPAC style) name:

N{A1}-acetyl, N{B1}-acetyl, N{Epsilon-B29}-[(4S)-4-carboxy-4-(17-carboxyheptadecanoylamino)butanoyl]-[GluA14,HisB25],des-ThrB27,ThrB30-Insulin (human).

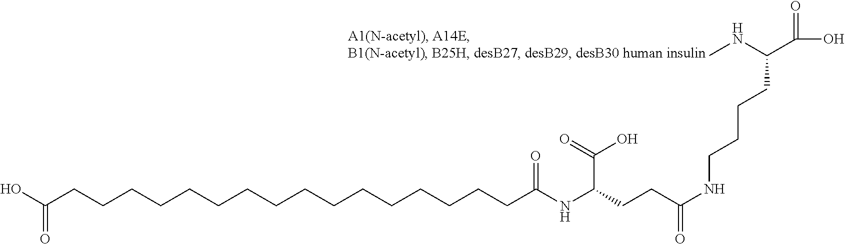 [SMILES: CN[C@@H](CCCCNC(=O)CC[C@H](NC(=O)CCCCCCCCCCCCCCCCC(=O)O)C(=O)O)C(=O)O]

A14E, B25H, desB27, B29K(N^{ε}octadecanedioyl-gGlu), desB30 human insulin (1 g) was dissolved in H₂O/DMSO ((2/1), 30 mL) and N,N-diisopropylethylamine (DIPEA) 100 uL was added to pH 7.9. Acetic acid N-hydroxysuccinimide ester (79 mg) dissolved in acetonitrile (10 mL) was added in portions over 15 min, pH changed to 10.8 during the addition. After 2 hours, the mixture was acidified to 1.7 by dropwise addition of hydrochloric acid (4 M) and the resulting mixture was purified by preparative HPLC. The pure fractions were pooled and lyophilised. The resulting product was dissolved in water and pH adjusted to 7.8 by means of 1N NaOH and lyophilised This afforded 160 mg of the title insulin.

LC-MS (electrospray): (m+4)/4: 1518.71; calcd: 1518.8.

### Example 34

### General Procedure (C)

### A1(N^{α}Acetyl), A14E, B1(N^{α}Acetyl), B25H, B29K(N^{ε}octadecandioyl-gGlu-2xOEG), desB30 human insulin

IUPAC (OpenEye, IUPAC style) name:

N{A1}-acetyl,N{B1}-acetyl,N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]-acetyl]-[GluA14,HisB25],des-ThrB30-Insulin (human).

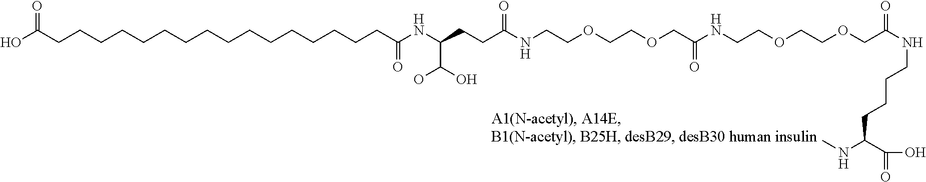 [SMILES: CN[C@@H](CCCCNC(=O)COCCOCCNC(=O)COCCOCCNC(=O)CC[C@H](NC(=O)CCCCCCCCCCCCCCCCC(=O)O)C(O)O)C(=O)O]

This compound was prepared as described above.

A14E, B25H, B29K(N^{ε}octadecandioyl-gGlu-2xOEG), desB30 human insulin (500 mg) was treated with acetic acid N-hydroxysuccinimide ester (37 mg) for 3.5 h. pH was subsequently adjusted to 1.5 followed by preparative HPLC purification. Lyophilisation followed by pH adjustment to 7.8 and lyophilisation afforded 184 mg of the title insulin.

LC-MS (electrospray): (m+4)/4: 1616.9; calcd: 1616.6.

### Example 35

### General Procedure (C)

### A1(N^{α}Dimethylglycyl), A14E, B1(N^{α}Dimethylglycyl), B25H, B29K(N^{ε}octadecanedioyl-gGlu-2xOEG), desB30 human insulin

IUPAC (OpenEye, IUPAC style) name:

N{A-1},N{A-1}-dimethyl, N{B-1},N{B-1}-dimethyl, N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]-ethoxy]ethoxy]acetyl]-GlyA-1,GlyB-1[GluA14,HisB25],des-ThrB30-Insulin (human).

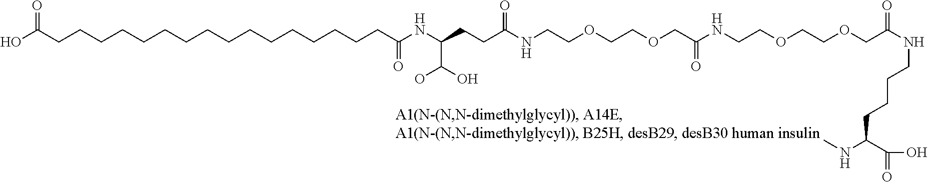 [SMILES: CN[C@@H](CCCCNC(=O)COCCOCCNC(=O)COCCOCCNC(=O)CC[C@H](NC(=O)CCCCCCCCCCCCCCCCC(=O)O)C(O)O)C(=O)O]

A14E, B25H, B29K(N^{ε}Octadecanedioyl-gGlu-OEG-OEG), desB30 human insulin (0.3 g) was dissolved in acetonitrile (4 mL) and diluted with water to 15 mL (pH=8). N,N-dimethylglycine N-hydroxysuccinimide ester (38 mg, prepared as described below) dissolved in acetonitrile was added dropwise, and the mixture was stirred for 100 minutes and a few drops methylamine was added. The mixture was acidified with acetic acid glacial and purified by HPLC. This afforded the title insulin.

LC-MS (electrospray): (m+4)/4: 1638; calcd: 1638.

N,N-dimethylglycine N-hydroxysuccinimide ester:

N,N-dimethylglycine (25 mg) and O—(N-succinimidyl)-1,1,3,3-tetramethyl uranium tetrafluoroborate (TSTU, 69 mg) was mixed with acetonitrile (2 mL), and N,N-diisopropylethylamine 46 uL was added. The mixture was gently heated until a solution was formed. This mixture was used directly, without further characterisation, in the acylation reaction.

### Example 36

### General Procedure (C)

### A1(N^{α}3-(N,N-Dimethylamino)propionyl), A14E, B1 (N^{α}3-(N,N-dimethylamino)propionyl), B25H, B29K(N^{ε}octadecanedioyl-gGlu-2xOEG), desB30 human insulin

IUPAC (OpenEye, IUPAC style) name:

N{A1}-3-(dimethylamino)propanoyl,N{B1}-3-(dimethylamino)propanoyl,N{Epsilon-B29}-[2-[2-[2-[[2-[2-[2-[[(4S)-4-carboxy-4-(17-carboxyheptadecanoylamino)butanoyl]amino]-ethoxy]ethoxy]acetyl]amino]ethoxy]ethoxy]acetyl]-[GluA14, HisB25],des-ThrB30-Insulin (human)

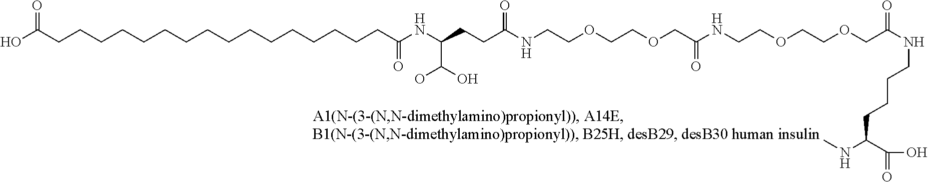 [SMILES: CN[C@@H](CCCCNC(=O)COCCOCCNC(=O)COCCOCCNC(=O)CC[C@H](NC(=O)CCCCCCCCCCCCCCCCC(=O)O)C(O)O)C(=O)O]

3-N,N-Dimethylaminopropionic acid (96 mg) was dissolved with TSTU (186 mg) in acetonitrile (10 mL). DIPEA was added to pH>8 and the mixture stirred at RT for 30 min. The resulting mixture was then added to a solution of A14E, B25H, B29K(N^{ε}octadecanedioyl-gGlu-2xOEG), desB30 human insulin (500 mg) dissolved in water/acetonitrile ((1/1), 20 mL). pH was adjusted to 7.9 with 1N NaOH and the resulting mixture was stirred gently at RT for 30 min. Subsequently, pH was raised to 10.3 for 5 min using 1N NaOH followed by acidification with 4N hydrochloric acid to pH 1.3. The resulting mixture was purified by preparative HPLC. Pure fractions were pooled, lyophilised to afford 17 mg of the title insulin.

LC-MS (electrospray): (m+4)/4: 1645.1; calcd: 1645.2.

### Example 37

### General Procedure (C)

### A1 (N^{α}4-(N,N-Dimethylamino)butanoyl), A14E, B1(N^{α}4-(N,N-dimethylamino)butanoyl), B25H, B29K(N^{ε}octadecanedioyl-gGlu-2xOEG), desB30 human insulin

IUPAC (OpenEye, IUPAC style) name:

N{A1}-4-(dimethylamino)butanoyl,N{B1}-4-(dimethylamino)butanoyl,N{Epsilon-B29}-[2-[2-[2-[[2-[2-[2-[[(4S)-4-carboxy-4-(17-carboxyheptadecanoylamino)butanoyl]amino]ethoxy]ethoxy]-acetyl]amino]ethoxy]ethoxy]acetyl]-[GluA14,HisB25],des-ThrB30-Insulin (human).

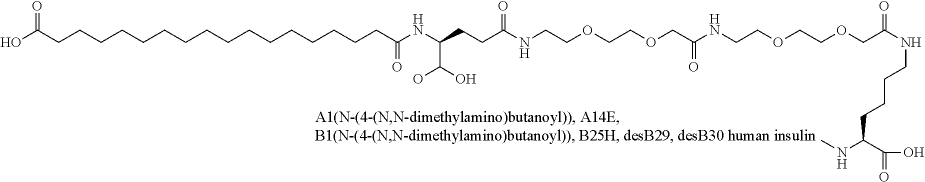 [SMILES: CN[C@@H](CCCCNC(=O)COCCOCCNC(=O)COCCOCCNC(=O)CC[C@H](NC(=O)CCCCCCCCCCCCCCCCC(=O)O)C(O)O)C(=O)O]

4-(N,N-Dimethylamino)butanoic acid (100 mg) was mixed with TSTU (178 mg) in acetonitrile (10 mL) DIPEA was added dropwise to pH 8 and the mixture was stirred for 1 h at RT. This resulted in a brownish liquid which was concentrated in vacuo to an oil. This was subsequently dissolved in acetonitrile (10 mL) and added to a solution of A14E, B25H, B29K(N^{ε}octadecanedioyl-gGlu-2xOEG), desB30 human insulin (420 mg) dissolved in water., pH was 7.8 changing to 6.3 after 30 min reaction. The solution was then acidified to pH 2.5 with addition of 1N hydrochloric acid dropwise and the resulting solution was purified by preparative HPLC. Pure fractions were pooled and lyophilised followed by dissolution in water and pH adjusted to 7.9. After a final lyophilisation 100 mg of the title insulin was obtained.

LC-MS (electrospray): (m+4)/4: 1652.0; calcd: 1652.2.

### Example 38

### General Procedure (C)

### A1(N^{α}3-(1-Piperidinyl)propionyl), A14E, B1(Nα3-(1-piperidinyl)propionyl), B25H, B29K(N^{ε}octadecanedioyl-gGlu-2xOEG), desB30 human insulin

IUPAC (OpenEye, IUPAC style) name:

N{A1}-3-piperidin-1-ylpropanoyl,N{B1}-3-piperidin-1-ylpropanoyl,N{Epsilon-B29}-[2-[2-[2-[[2-[2-[2-[[(4S)-4-carboxy-4-(17-carboxyheptadecanoylamino)butanoyl]amino]ethoxy]ethoxy]-acetyl]amino]ethoxy]ethoxy]acetyl]-[GluA14,HisB25],des-ThrB30-Insulin (human).

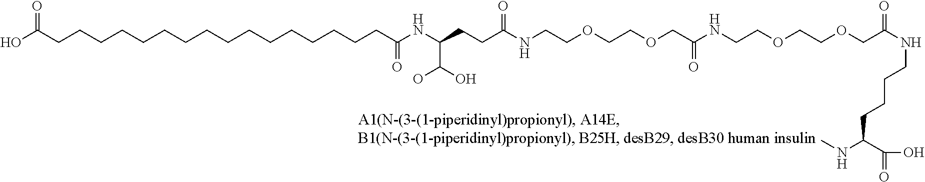 [SMILES: CN[C@@H](CCCCNC(=O)COCCOCCNC(=O)COCCOCCNC(=O)CC[C@H](NC(=O)CCCCCCCCCCCCCCCCC(=O)O)C(O)O)C(=O)O]

3-(1-Piperidinyl)propionic acid (98.5 mg) was dissolved with TSTU (188 mg) in acetonitrile (20 mL), pH was adjusted to 8 with dropwise addition of DIPEA. The mixture was stirred at RT for 30 min then evaporated to an oil which was re-dissolved in acetonitrile (10 mL) and added to a solution of A14E, B25H, B29K(N^{ε}octadecanedioyl-gGlu-2xOEG), desB30 human insulin (500 mg) in water (20 mL). pH was 7.4 changing to 6.7 after the addition of the activated acid. After stirring at RT for 15 min pH was adjusted to 10.2 by addition of 1N NaOH and the mixture was stirred for 5 min. Subsequently the mixture was acidified to pH 1 by dropwise addition of 4N hydrochloric acid. The resulting mixture was purified by preparative HPLC. The pure fractions were pooled and lyophilised followed by dissolution in water, pH was adjusted to 7.9 by means of 1N NaOH. Lyophilisation afforded 111 mg of the title insulin.

LC-MS (electrospray): (m+4)/4: 1665.2; calcd: 1665.2.

### Example 39

### General Procedure (C)

### A1(N^{α} Dimethylglycyl), A14E, B1(N^{α}Dimethylglycyl), B25H, desB27, B29K(N^{ε}octadecanedioyl-gGlu), desB30 human insulin

IUPAC (OpenEye, IUPAC style) name:

N{A-1},N{A-1}-dimethyl,N{B-1},N{B-1}-dimethyl,N{Epsilon-B29}-[(4S)-4-carboxy-4-(17-carboxyheptadecanoylamino)butanoyl]-GlyA-1,GlyB-1[GluA14,HisB25],des-ThrB27,ThrB30-Insulin (human).

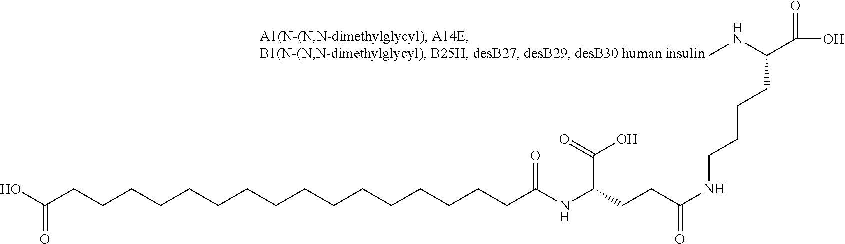 [SMILES: CN[C@@H](CCCCNC(=O)CC[C@H](NC(=O)CCCCCCCCCCCCCCCCC(=O)O)C(=O)O)C(=O)O]

A14E, B25H, desB27, B29K(N^{ε}octadecanedioyl-gGlu), desB30 human insulin (1.1 g) was dissolved in water (40 mL) and acetonitrile (10 mL), pH of the resulting solution was 7.5. Crude dimethylaminoacetic acid 2,5-dioxopyrrolidin-1-yl ester, prepared as described above, (294 mg) was added under vigorous stirring and the resulting mixture was further stirred for 1 h at RT. Methylamine (few drops) was added and pH adjusted to 12 with 1N NaOH. After 30 min pH was adjusted to 4 with acetic acid and the mixture was purified by preparative HPLC. The pure fractions were pooled and lyophilised, followed by dissolution in water and pH adjustment to 7.8 by means of 0.1N NaOH. Lyophilisation afforded 517 mg of the title insulin.

LC-MS (electrospray): (m+4)/4: 1540.0; calcd: 1540.29.

### Example 40

### General Procedure (C)

### A1G(N^{α}acetyl), A14E, B1F(N^{α}acetyl), B25H, desB27, B29K(N^{ε}octadecanedioyl-gGlu-2xOEG), desB30 human insulin

IUPAC (OpenEye, IUPAC style) name:

N{A1}-acetyl, N{B1}-acetyl, N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]-[GluA14,HisB25],des-ThrB27,ThrB30-Insulin (human)

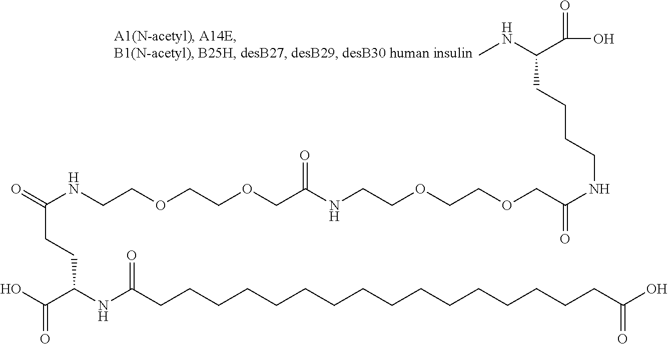 [SMILES: CN[C@@H](CCCCNC(=O)COCCOCCNC(=O)COCCOCCNC(=O)CC[C@H](NC(=O)CCCCCCCCCCCCCCCCC(=O)O)C(=O)O)C(=O)O]

LC-MS (electrospray): m/z=1594 (M+1)/4; calcd: 1591.

### Example 41

### General Procedure (C)

### A1G(N^{α}2-Picolyl), A14E, B1F(N^{α}2-Picolyl), B25H, desB27, B29K(N(eps)octadecanedioyl-gGlu-2xOEG), desB30 human insulin

IUPAC (OpenEye, IUPAC style) name:

N{A1}-pyridine-2-carbonyl,N{B1}-pyridine-2-carbonyl, N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]-ethoxy]ethoxy]acetyl]-[GluA14,HisB25],des-ThrB27,ThrB30-Insulin (human)

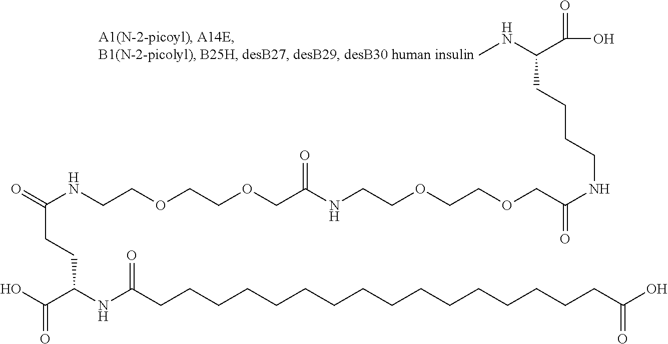 [SMILES: CN[C@@H](CCCCNC(=O)COCCOCCNC(=O)COCCOCCNC(=O)CC[C@H](NC(=O)CCCCCCCCCCCCCCCCC(=O)O)C(=O)O)C(=O)O]

This analogue was prepared similarly as described above using 2-picolinic acid N-hydroxysuccinimide ester as acylation reagent.

LC-MS (electrospray): (m+4)/4: 1622.74; calcd: 1622.88.

The analogues in the following examples may be prepared similarly:

### Example 42

### General Procedure (C)

### A1(N^{α}Acetyl), A14E, B1(N^{α}Acetyl), B25H, B29K(N^{ε}eicosanedioyl-gGlu), desB30 human insulin

IUPAC (OpenEye, IUPAC style) name:

N{A1}-acetyl,N{B1}-acetyl,N{Epsilon-B29}-[(4S)-4-carboxy-4-(19-carboxynonadecanoylamino)butanoyl]-[GluA14,HisB25],des-ThrB30-Insulin (human)

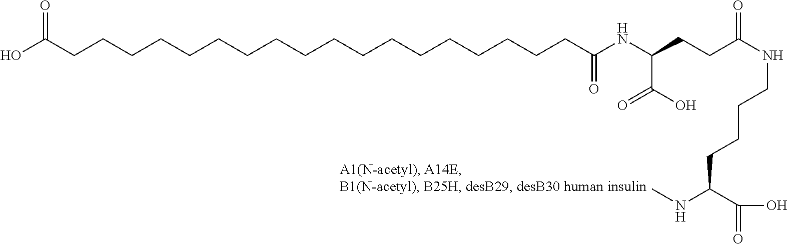 [SMILES: CN[C@@H](CCCCNC(=O)CC[C@H](NC(=O)CCCCCCCCCCCCCCCCCCC(=O)O)C(=O)O)C(=O)O]

### Example 43

### General Procedure (C)

### A1(N^{α}Acetyl), A14E, B1(N^{α}Acetyl), B25H, B29K(N^{ε}eicosanedioyl-gGlu-2xOEG), desB30 human insulin

IUPAC (OpenEye, IUPAC style) name:

N{A1}-acetyl, N{B1}-acetyl, N{Epsilon-B29}-[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]-acetyl]-[GluA14,HisB25],des-ThrB30-Insulin (human)

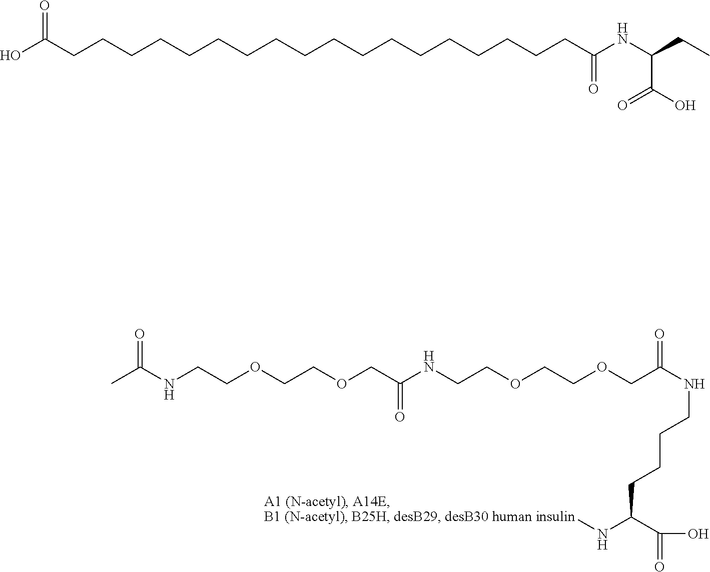

### Example 44

### General Procedure (C)

### A1(N^{α}Acetyl), A14E, B1(N^{α}Acetyl), B16H, B25H, B29K(N^{ε}eicosanedioyl-gGlu-2xOEG), desB30 human insulin

IUPAC (OpenEye, IUPAC style) name:

N{A1}-acetyl, N{B1}-acetyl, N{Epsilon-B29}-[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]-acetyl]-[GluA14,HisB16,HisB25],des-ThrB30-Insulin (human)

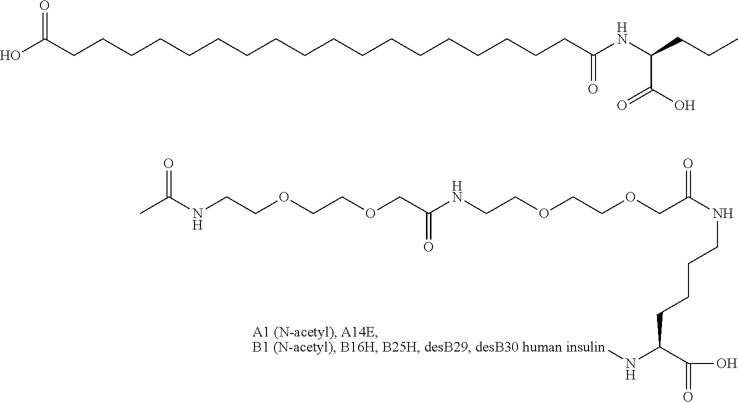

### Example 45

### General Procedure (C)

### A1(N^{α}Acetyl), A14E, B1(N^{α}Acetyl), B16H, B25H, B29K(N^{ε}eicosanedioyl-gGlu), desB30 human insulin

IUPAC (OpenEye, IUPAC style) name:

N{A1}-acetyl, N{B1}-acetyl, N{Epsilon-B29}-[(4S)-4-carboxy-4-(19-carboxynonadecanoylamino)butanoyl]-[GluA14,HisB16,HisB25],des-ThrB30-Insulin (human)

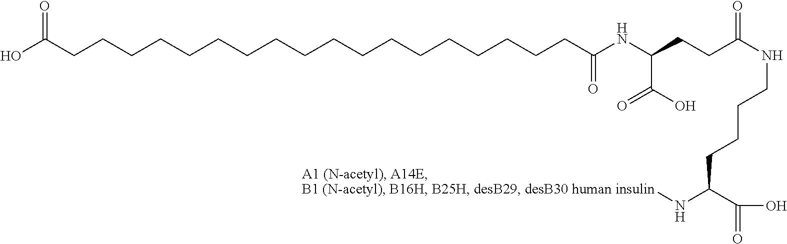

### Example 46

### General Procedure (C)

### A1(N^{α}Dimethylglycyl), A14E, B1(N^{α}Dimethylglycyl), B16H, B25H, B29K(N^{ε}hexadecanedioyl-gGlu), desB30 human insulin

IUPAC (OpenEye, IUPAC style) name:

N{A-1}, N{A-1}-dimethyl,N{B-1}, N{B-1}-dimethyl, N{Epsilon-B29}-[(4S)-4-carboxy-4-(15-carboxypentadecanoylamino)butanoyl]-GlyA-1,GlyB-1[GluA14,HisB16,HisB25],des-ThrB30-Insulin (human)

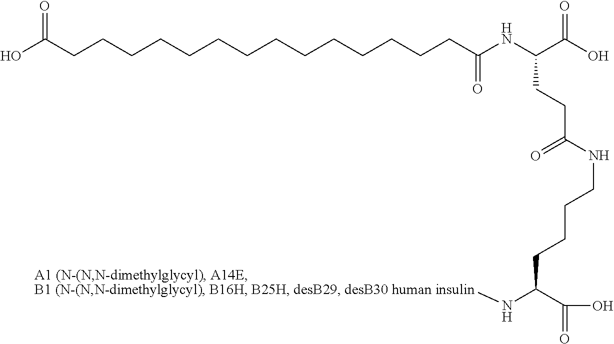

### Example 47

### General Procedure (C)

### A-1(N^{α}Trimethyl), A14E, B-1(N^{α}Trimethyl), B25H, B29K(N^{ε}octadecanedioyl-gGlu-2xOEG), desB30 human insulin

IUPAC (OpenEye, IUPAC style) name:

N{A1}-[2-(trimethylazaniumyl)acetyl], N{B1}-[2-(trimethylazaniumyl)acetyl], N{Epsilon-B29}-[2-[2-[2-[[2-[2-[2-[[(4S)-4-carboxy-4-(17-carboxyheptadecanoylamino)butanoyl]amino]-ethoxy]ethoxy]acetyl]amino]ethoxy]ethoxy]acetyl]-[GluA14, HisB25],des-ThrB30-Insulin (human)

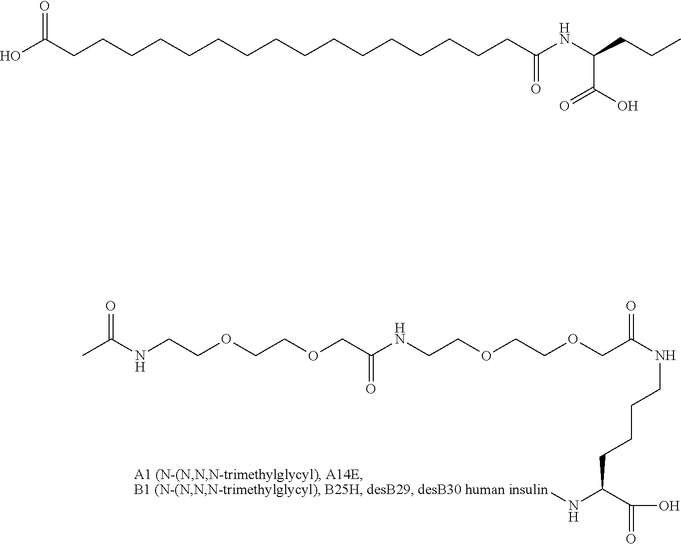

This analogue may be prepared similarly as the A1,B1-diacetyl analogues using N,N,N-trimethylglycine OSu ester as acylation reagent.

### Example 48

### General Procedure (C)

### A1(N^{α}Acetyl), A14E, B1(N^{α}Acetyl), desB27, B29K(N^{ε}octadecanedioyl-gGlu), desB30 human insulin

IUPAC (OpenEye, IUPAC style) name:

N{A1}-acetyl, N{B1}-acetyl, N{Epsilon-B29}-[(4S)-4-carboxy-4-(17-carboxyheptadecanoylamino)butanoyl]-[GluA14],des-ThrB27,ThrB30-Insulin (human)

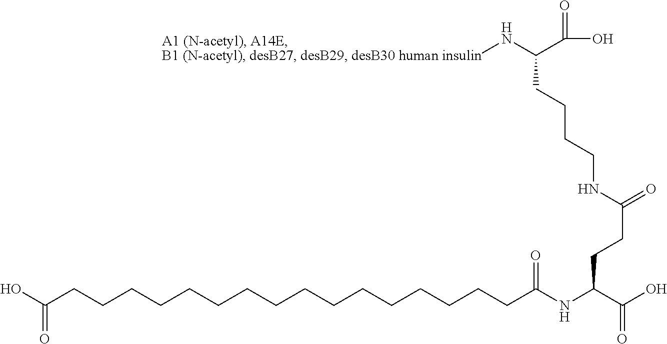

### Example 49

### General Procedure (C)

### A1(N^{α}Acetyl), A14E, B1(N^{α}Acetyl), desB27, B29K(N^{ε}octadecanedioyl-gGlu-2xOEG), desB30 human insulin

IUPAC (OpenEye, IUPAC style) name:

N{A1}-acetyl, N{B1}-acetyl, N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]-acetyl]-[GluA14],des-ThrB27,ThrB30-Insulin (human)

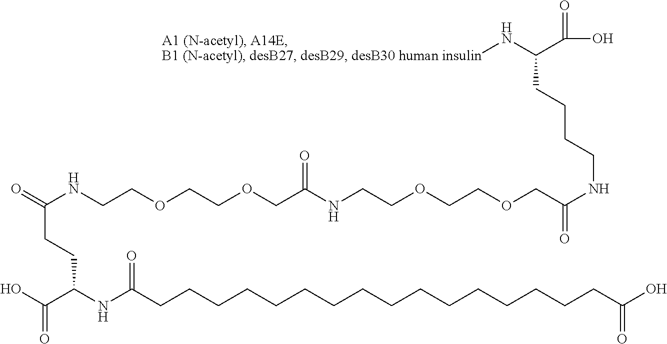

### Example 50

### General Procedure (C)

### A1(N^{α}Acetyl), A14E, B1(N^{α}Acetyl), B25H, B29K(N^{ε}octadecanedioyl-gGlu), desB30 human insulin

IUPAC (OpenEye, IUPAC style) name:

N{A1}-acetyl, N{B1}-acetyl, N{Epsilon-B29}-[(4S)-4-carboxy-4-(17-carboxyheptadecanoylamino)butanoyl-[GluA14, HisB25],des-ThrB30-Insulin (human)

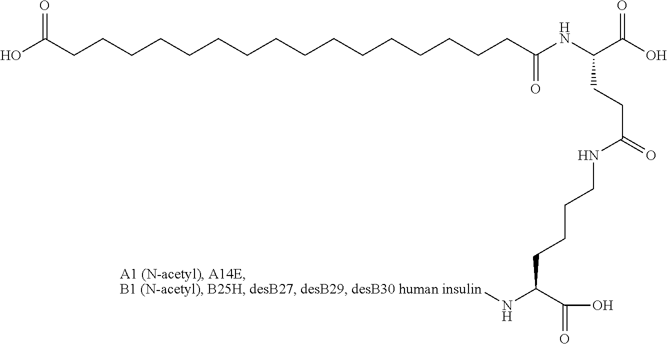

### Example 51

### General Procedure (C)

### A1G(N^{α}Acetyl), A14E, B1F(N^{α}Acetyl), desB27, B29K(N^{ε}eicosanedioyl-gGlu), desB30 human insulin

IUPAC (OpenEye, IUPAC style) name:

N{A1}-acetyl,N{B1}-acetyl,N{Epsilon-B29}-[(4S)-4-carboxy-4-(19-carboxynonadecanoylamino)butanoyl]-[GluA14],des-ThrB27,ThrB30-Insulin (human)

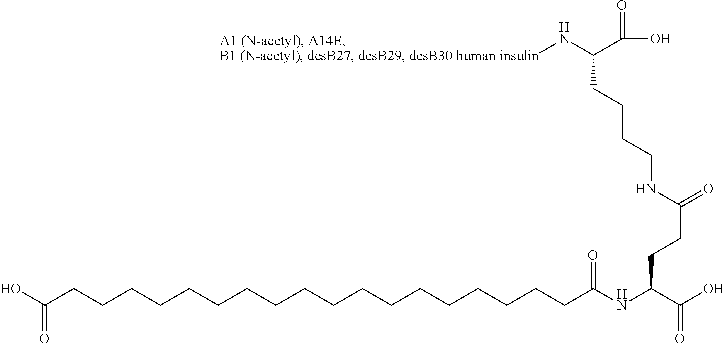

### Example 52

### General Procedure (C)

### A1G(N^{α}Acetyl), A14E, B1F(N^{α}Acetyl), desB27, B29K(N^{ε}eicosanedioyl-gGlu-2xOEG), desB30 human insulin

IUPAC (OpenEye, IUPAC style) name:

N{A1}-acetyl, N{B1}-acetyl, N{Epsilon-B29}-[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]acetyl]-[GluA14],des-ThrB27,ThrB30-Insulin (human)

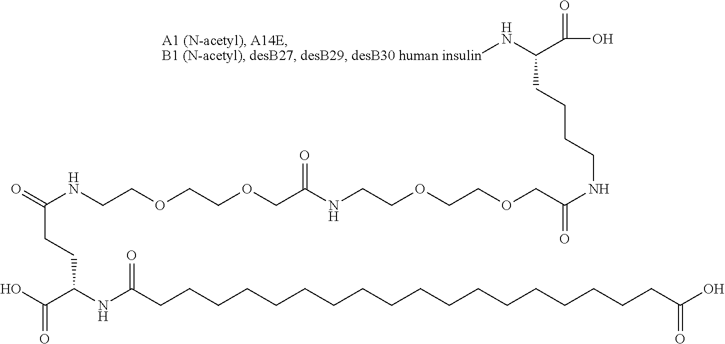

### Example 53

### General Procedure (C)

### A1G(N^{α}Acetyl), A14E, B1F(N^{α}Acetyl), B25H, desB27, B29K(N^{ε}eicosanedioyl-gGlu⁻²xOEG), desB30 human insulin

IUPAC (OpenEye, IUPAC style) name:

N{A1}-acetyl, N{B1}-acetyl, N{Epsilon-B29}-[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]acetyl]-[GluA14,HisB25],des-ThrB27,ThrB30-Insulin (human)

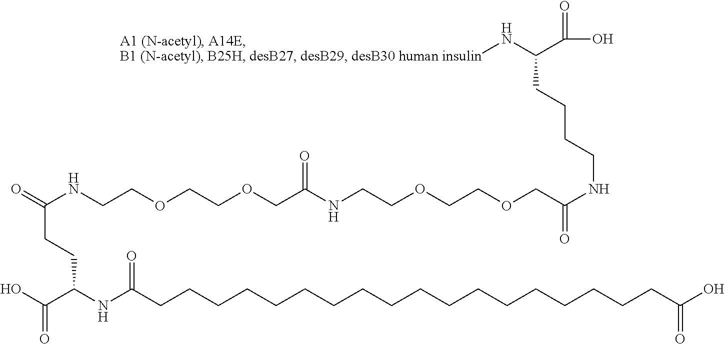

### General Procedure (D) for Preparation for N-Terminal Acylation of Acylated Insulins of this Invention Using (Cyclic) Carboxylic Acid Anhydrides

The lysine-acylated insulin is dissolved in a polar aprotic solvent, optionally containing an organic base, such as triethyl amine or N,N-diisopropylethylamine and an excess of acylation reagent, eg. as succinic or glutaric acid anhydride is added. The mixture is allowed to stand to completion of the reaction. If necessary, more acylation reagent is added. The product is isolated, eg. by preparative HPLC or by anion exchange chromatography.

The general procedure (D) is illustrated in the following example.

Alternatively, Procedure (D) can be performed in an aqueous media using N-hydroxysuccinimide activated diacids (or anhydrides) as illustrated in example 55.

### Example 54

### General Procedure (D)

### A1(N^{α}Succinyl), A14E, B1(N^{α}succinyl), B25H, desB27, B29K(N^{ε}octadecanedioyl-gGlu-2xOEG), desB30 human insulin

IUPAC (OpenEye, IUPAC style) name:

N{A1}-3-carboxypropanoyl,N{B1}-3-carboxypropanoyl,N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]-ethoxy]ethoxy]acetyl]-[GluA14,HisB25],des-ThrB27,ThrB30-Insulin (human)

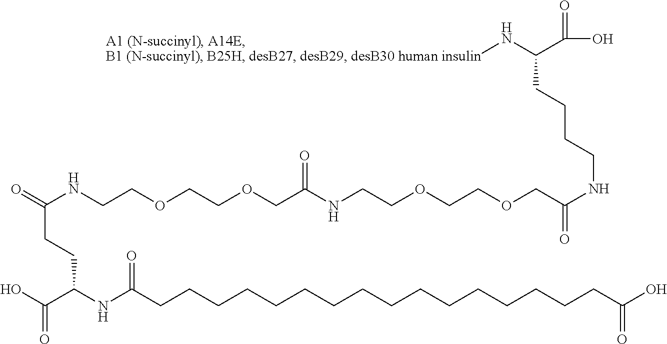

A14E, B25H, desB27, B29K(N^{ε}octadecanedioyl-gGlu-2xOEG), desB30 human insulin (WO 2009/115469, example 57, 1 g) was dissolved in DMSO (15 mL) and added N,N-diisopropyl-ethylamine (DIPEA, 136 μL) and the resulting mixture was allowed to stand for 30 minutes. Succinic anhydride (40 mg) was added and the resulting mixture was stirred gently for 1 hour. The mixture was diluted with water (150 mL) and ethanol (150 mL) and pH was adjusted to 8 with 1N hydrochloric acid. The product was purified by anion exchange chromatography:

A buffer: 15 mM TRIS, 30 mM Ammonium acetate in 50% ethanol, pH 8 (1.25 mS/cm)

B buffer: 15 mM TRIS, 300 mM Ammonium acetate in 50% ethanol, pH 8 (8 mS/cm)

Column: 30×250 mm, Source 30Q (180 g)

Flow: 40 mL/min

The column was equilibrated with A buffer. The mixture was applied to the column and was eluted with 2 CV A buffer followed by a gradient of 0-80% B over 30 minutes. The fraction containing the product was concentrated in vacuo to approximately 100 mL and the product was precipitated by pH adjustment to 4.9 with 1N hydrochloric acid. The precipitate was isolated by centrifugation, washed with a little water, and dissolved in 30% acetonitrile/water (100 mL). pH was adjusted to 8.0 with 1N sodium hydroxide and the mixture was lyophilised. This afforded 620 mg (60%) of the title compound.

LC-MS (electrospray): m/z=1620 (M+1)/4; calcd: 1620.

### Example 55

### General Procedure (D)

### A1(N^{α}Succinyl), A14E, B1(N^{α}succinyl), B25H, B29K(N^{ε}octadecanedioyl-gGlu-2xOEG), desB30 human insulin

IUPAC (OpenEye, IUPAC style) name:

N{A1}-3-carboxypropanoyl,N{B1}-3-carboxypropanoyl, N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]-ethoxy]ethoxy]acetyl]-[GluA14,HisB25],des-ThrB30-Insulin (human)

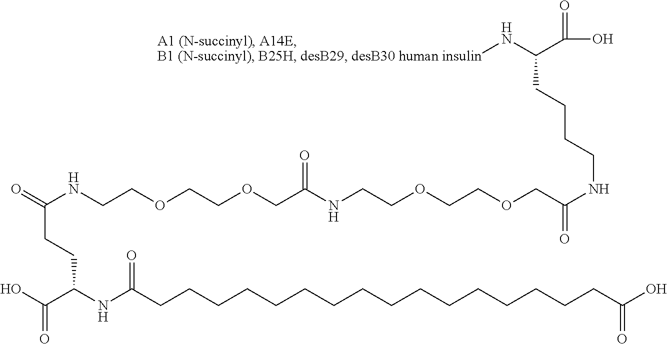

### This Analogue was Prepared by an Aqueous Method Similarly as Described Above

To succinic acid (10 mg) dissolved in THF/DMF 1:1 (0.5 ml) was added TSTU (30 mg) and DIPEA (0.02 ml). The mixture was left at RT for 2 h before half of the mixture was added to a solution of A14E, B25H, B29K(N^{ε}Octadecanedioyl-gGlu-2xOEG), desB30 human insulin (0.1 g) in 0.1M NaHCO₃ (1 ml) adjusted to pH 9.3 with 1M NaOH. After gently stirring for 2 h the other half of the OSu-activated succinic acid was added. After 4 h pH was adjusted to 7 with 1M HCl. The title compound was isolated by RP HPLC:

Column: Phenomenex, Gemini, 5μ, C18, 110 Å, 250×20 cm

Flow: 10 ml/min

Eluent:
A: 10 mM Tris, 15 mM ammonium sulfate, 20% CH₃CN, pH 7.3
B: 20% water in CH₃CN

Gradient:
0-7.5 min: 0% B
7.5-47.5 min: 0% B to 40% B
47.5-52.5 min: 40% B
52.5-57.5 min: 40% B to 100% B
57.5-60 min: 100% B
60-63 min: 0% B

Pure fractions were pooled and lyophilized. The dry material was dissolved in 0.1% TFA in water and CH₃CN and was desalted by RP HPLC.

Column: Phenomenex, Gemini, 5μ, C18, 110 Å, 250×20 cm

Flow: 10 mL/min

Eluent: A: 0.1% TFA in water B: 0.1% TFA in CH₃CN

Gradient:
0-7.5 min: 25% B
7.5-37.5 min: 25% B to 60% B
37.5-42.5 min: 60% B
42.5-48 min: 60% B to 100% B
48-50 min: 100% B
50-53 min: 25% B

MALDI-MS: m/z: 6580.0; calcd: 6578.5.

LC-MS (electrospray): (m+4)/4: 1645.68 (6578.7)

### Example 56

### General Procedure (D)

### A1(N^{α}Succinyl), A14E, B1(N^{α}succinyl), desB27, B29K(N^{ε}octadecanedioyl-gGlu-2xOEG), desB30 human insulin

IUPAC (OpenEye, IUPAC style) name:

N{A1}-3-carboxypropanoyl,N{B1}-3-carboxypropanoyl, N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]-ethoxy]ethoxy]acetyl]-[GluA14],des-ThrB27,ThrB30-Insulin (human)

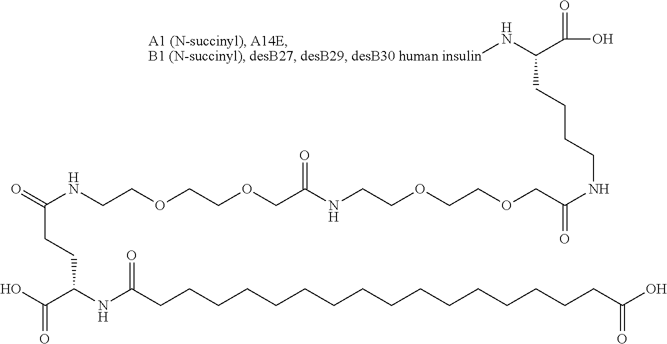

This analogue was prepared similarly as described above

LC-MS (electrospray): m/z=1623 (M+1)/4; calcd: 1623.

### Example 57

### General Procedure (D)

### A1(N^{α}Glutaryl), A14E, B1 (N^{α}glutaryl), B25H, B29K(N^{ε}octadecanedioyl-gGlu-2xOEG), desB30 human insulin

IUPAC (OpenEye, IUPAC style) name:

N{A1}-4-carboxybutanoyl,N{B1}-4-carboxybutanoyl,N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]-ethoxy]ethoxy]acetyl]-[GluA14,HisB25],des-ThrB30-Insulin (human)

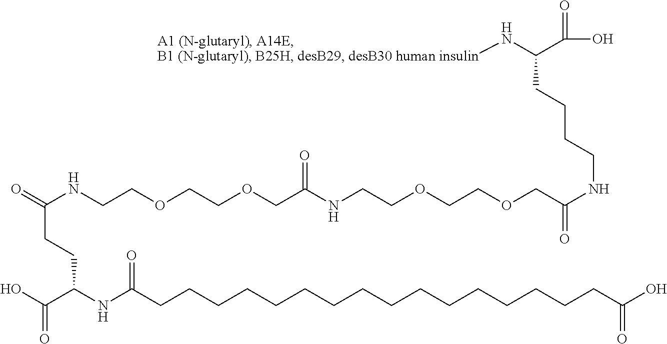

This analogue was prepared similarly as described above

LC-MS (electrospray): m/z=1623 (M+1)/4; calcd: 1623.

### Example 58

### General Procedure (D)

### A1(N^{α}Glutaryl), A14E, B1(N^{α}glutaryl), desB27, B29K(N^{ε}octadecanedioyl-gGlu-2xOEG), desB30 human insulin

IUPAC (OpenEye, IUPAC style) name:

N{A1}-4-carboxybutanoyl, N{B1}-4-carboxybutanoyl,N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]-ethoxy]ethoxy]acetyl]-[GluA14],des-ThrB27,ThrB30-Insulin (human)

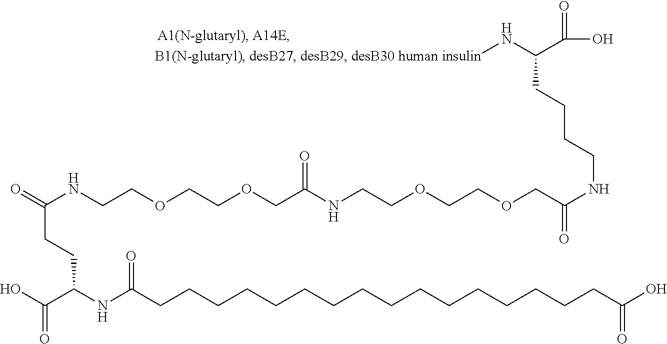

LC-MS (electrospray): m/z=1630 (M+1)/4; calcd: 1630.

### Example 59

### General Procedure (D)

### A1(N^{α}Diglycolyl), A14E, B1(N^{α}diglycolyl), B25H, desB27, B29K(N^{ε}octadecanedioyl-gGlu-2xOEG), desB30 human insulin

IUPAC (OpenEye, IUPAC style) name:

N{A1}-[2-(carboxymethoxy)acetyl],N{B1}-[2-(carboxymethoxy)acetyl], N{Epsilon-B29}-[2-[2-[2-[[2-[2-[2-[[(4S)-4-carboxy-4-(17-carboxyheptadecanoylamino)butanoyl]amino]ethoxy]ethoxy]-acetyl]amino]ethoxy]ethoxy]acetyl]-[GluA14,HisB25],des-ThrB27,ThrB30-Insulin (human)

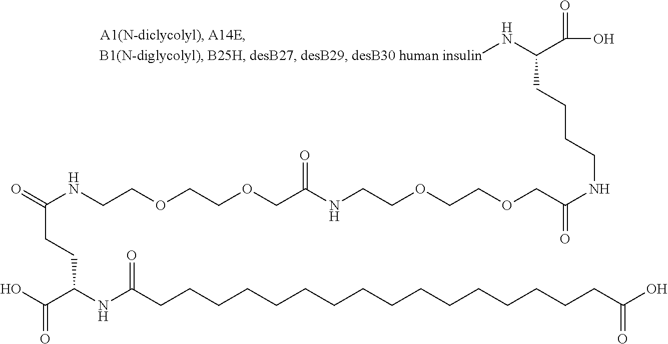

This analogue was prepared similarly as described above using diglycolic anhydride as acylation reagent.

LC-MS (electrospray): m/z=1628.14 (M+1)/4; calcd: 1628.35.

### Example 60

### General Procedure (D)

### A1(N^{α}Glutaryl), A14E, B1(N^{α}glutaryl), B25H, desB27, B29K(N^{ε}octadecanedioyl-gGlu-2xOEG), desB30 human insulin

IUPAC (OpenEye, IUPAC style) name:

N{A1}-4-carboxybutanoyl, N{B1}-4-carboxybutanoyl, N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]-ethoxy]ethoxy]acetyl]-[GluA14,HisB25],des-ThrB27,ThrB30-Insulin (human)

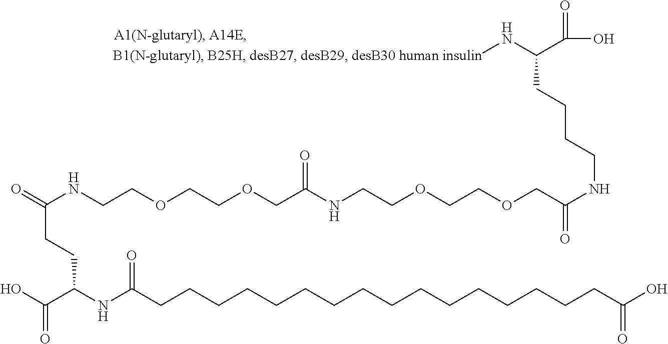

LC-MS (electrospray): m/z=1627.4 (M+1)/4; calcd: 1627.4.

### Example 61

### General Procedure (D)

### A1(N^{α}Succinyl), A14E, B1(N^{α}succinyl), desB27, B29K(N^{ε}octadecanedioyl-gGlu), desB30 human insulin

IUPAC (Open Eye, IUPAC style) name:

N{A1}-3-carboxypropanoyl,N{B1}-3-carboxypropanoyl, N{Epsilon-B29}-[(4S)-4-carboxy-4-(17-carboxyheptadecanoylamino)butanoyl]-[GluA14],des-ThrB27,ThrB30-Insulin (human)

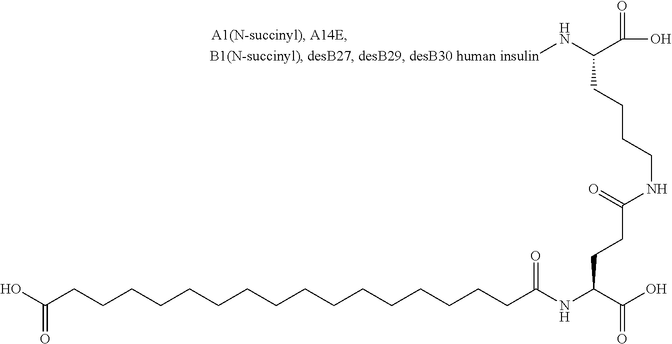

LC-MS (electrospray): m/z=1550.1 (M+1)/4; calcd: 1550.3.

The following analogues may be prepared similarly:

### Example 62

### General Procedure (D)

### A1(N^{α}Succinyl), A14E, B1(N^{α}succinyl), B25H, desB27, B29K(N^{ε}eicosanedioyl-gGlu-2xOEG), desB30 human insulin

IUPAC (OpenEye, IUPAC style) name:

N{A1}-3-carboxypropanoyl,N{B1}-3-carboxypropanoyl, N{Epsilon-B29}-[2-[2-[2-[[2-[2-[2-[[(4S)-4-carboxy-4-(19-carboxynonadecanoylamino)butanoyl]amino]ethoxy]ethoxy]acetyl]amino]-ethoxy]ethoxy]acetyl]-[GluA14,HisB25],des-ThrB27,ThrB30-Insulin (human)

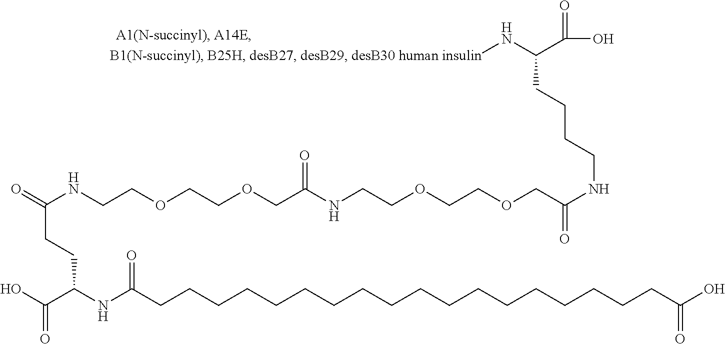

### Example 63

### General Procedure (D)

### A1(N^{α}Succinyl), A14E, B1(N^{α}succinyl), desB27, B29K(N^{ε}eicosanedioyl-gGlu-2xOEG), desB30 human insulin

IUPAC (OpenEye, IUPAC style) name:

N{A1}-3-carboxypropanoyl,N{B1}-3-carboxypropanoyl,N{Epsilon-B29}-[2-[2-[2-[[2-[2-[2-[[(4S)-4-carboxy-4-(19-carboxynonadecanoylamino)butanoyl]amino]ethoxy]ethoxy]acetyl]amino]-ethoxy]ethoxy]acetyl]-[GluA14],des-ThrB27,ThrB30-Insulin (human)

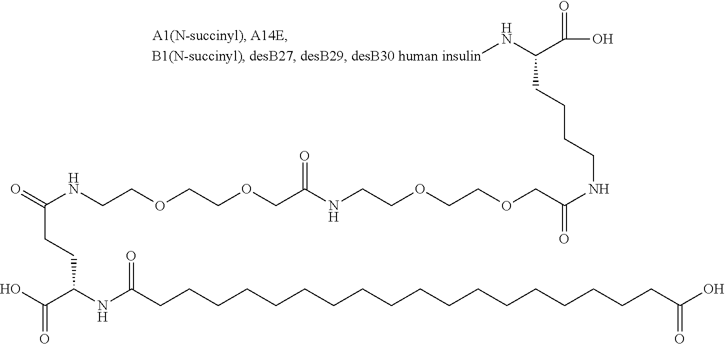

### Example 64

### General Procedure (D)

### A1(N^{α}Succinyl), A14E, B1(N^{α}succinyl), B16H, desB27, B29K(N^{ε}eicosanedioyl-gGlu-2xOEG), desB30 human insulin

IUPAC (OpenEye, IUPAC style) name:

N{A1}-3-carboxypropanoyl,N{B1}-3-carboxypropanoyl, N{Epsilon-B29}-[2-[2-[2-[[2-[2-[2-[[(4S)-4-carboxy-4-(19-carboxynonadecanoylamino)butanoyl]amino]ethoxy]ethoxy]acetyl]amino]-ethoxy]ethoxy]acetyl]-[GluA14, HisB16],des-ThrB27,ThrB30-Insulin (human)

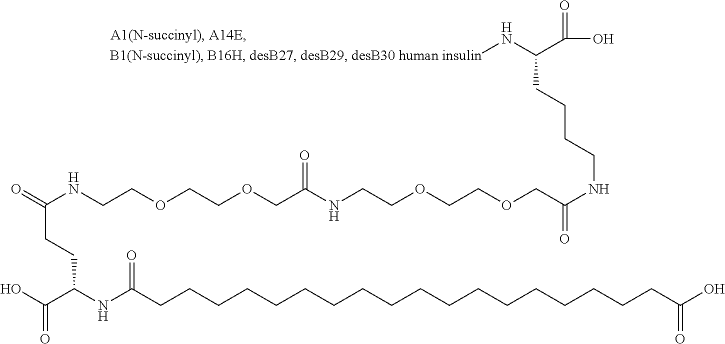

### Example 65

### General Procedure (D)

### A1(N^{α}Succinyl), A14E, B1(N^{α}succinyl), B25H, B29K(N^{ε}eicosanedioyl-gGlu-2xOEG), desB30 human insulin

IUPAC (OpenEye, IUPAC style) name:

N{A1}-3-carboxypropanoyl,N{B1}-3-carboxypropanoyl, N{Epsilon-B29}-[2-[2-[2-[[2-[2-[2-[[(4S)-4-carboxy-4-(19-carboxynonadecanoylamino)butanoyl]amino]ethoxy]ethoxy]acetyl]amino]-ethoxy]ethoxy]acetyl]-[GluA14,HisB25],des-ThrB30-Insulin (human)

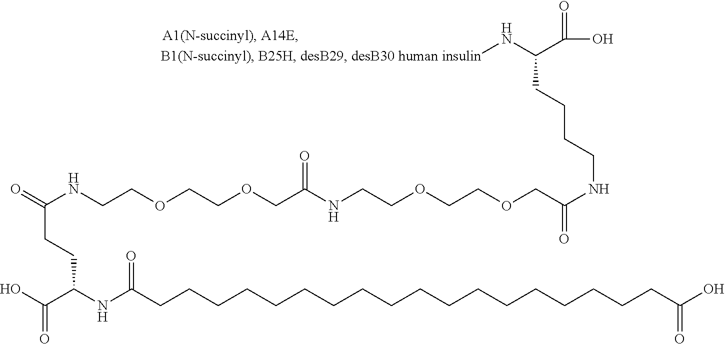

### Example 66

### General Procedure (D)

### A1(N^{α}Succinyl), A14E, B1(N^{α}succinyl), desB27, B29K(1N^{ε}eicosanedioyl-gGlu), desB30 human insulin

IUPAC (OpenEye, IUPAC style) name:

N{A1}-3-carboxypropanoyl,N{B1}-3-carboxypropanoyl,N{Epsilon-B29}-[(4S)-4-carboxy-4-(19-carboxynonadecanoylamino)butanoyl]-[GluA14],des-ThrB27,ThrB30-Insulin (human)

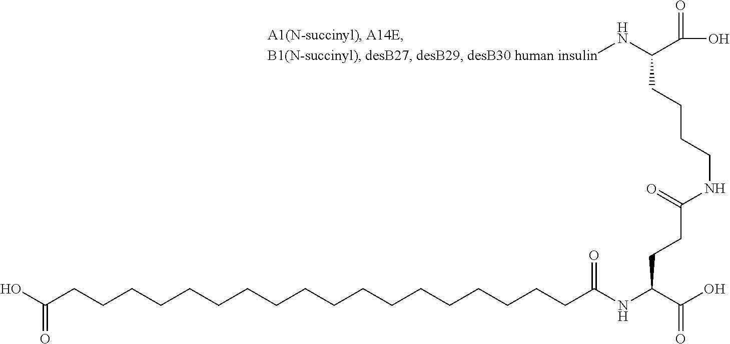

### Example 67

### General Procedure (D)

### A1(N^{α}Glutaryl), A14E, B1(N^{α}glutaryl), desB27, B29K(N^{ε}eicosanedioyl-gGlu), desB30 human insulin

IUPAC (OpenEye, IUPAC style) name:

N{A1}-4-carboxybutanoyl,N{B1}-4-carboxybutanoyl, N{Epsilon-B29}-[(4S)-4-carboxy-4-(19-carboxynonadecanoylamino)butanoyl]-[GluA14],des-ThrB27,ThrB30-Insulin (human)

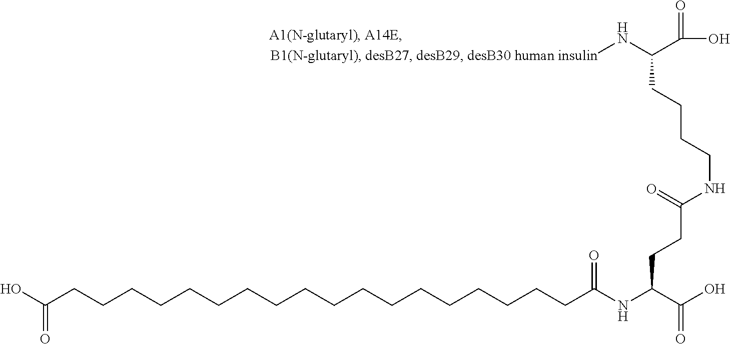

The following analogues were prepared similarly:

### Example 68

### General Procedure (D)

### A1(N^{α}Glutaryl), A14E, B1(N^{α}glutaryl), desB27, B29K(N^{ε}eicosanedioyl-gGlu-2xOEG), desB30 human insulin

IUPAC (OpenEye, IUPAC style) name:

N{A1}-4-carboxybutanoyl, N{B1}-4-carboxybutanoyl,N{Epsilon-B29}-[2-[2-[[(4S)-4-carboxy-4-(19-carboxynonadecanoylamino)butanoyl]amino]ethoxy]ethoxy]acetyl]amino]-ethoxy]ethoxy]acetyl]-[GluA14],des-ThrB27,ThrB30-Insulin (human)

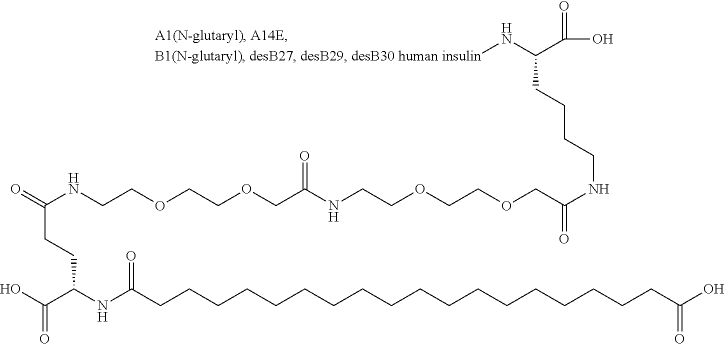

LC-MS (electrospray): m/z=1637.1 (M+1)/4; calcd: 1636.9.

### Example 69

### General Procedure (D)

### A1(N^{α}Glutaryl), A14E, B1(N^{α}glutaryl), B25H, desB27, B29K(N^{ε}eicosanedioyl-gGlu-2xOEG), desB30 human insulin

IUPAC (OpenEye, IUPAC style) name:

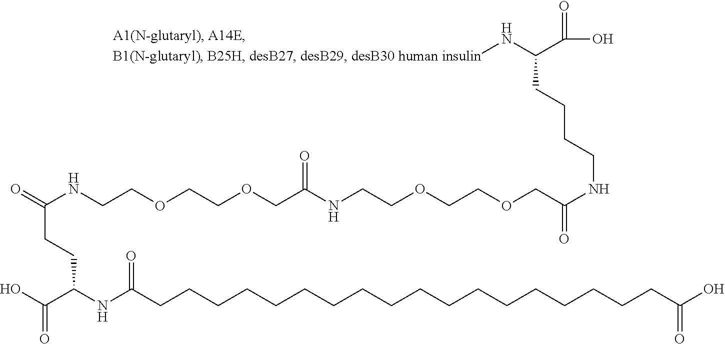

LC-MS (electrospray): m/z=1634.4 (M+1)/4; calcd: 1634.4.

The following analogues may be prepared similarly:

### Example 70

### General Procedure (D)

### A1(N^{α}Glutaryl), A14E, B1(N^{α}glutaryl), desB27, B29K(N^{ε}eicosanedioyl-gGlu-2xOEG), desB30 human insulin

IUPAC (OpenEye, IUPAC style) name:

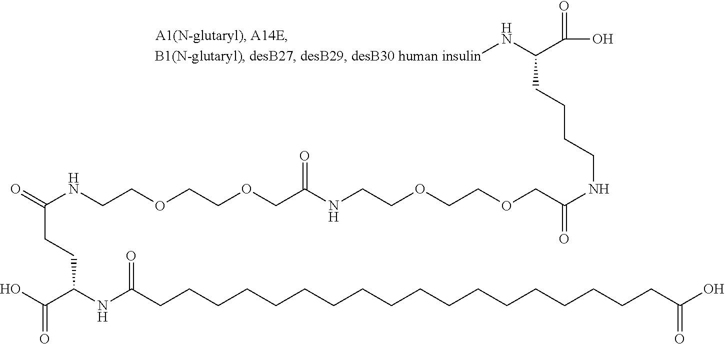

### Example 71

### General Procedure (D)

### A1(N^{α}Glutaryl), A14E, B1(N^{α}glutaryl), B25H, B29K(N^{ε}eicosanedioyl-gGlu-2xOEG), desB30 human insulin

IUPAC (OpenEye, IUPAC style) name:

N{A1}-4-carboxybutanoyl,N{B1}-4-carboxybutanoyl, N{Epsilon-B29}-[2-[2-[2-[[2-[2-[2-[[(4S)-4-carboxy-4-(19-carboxynonadecanoylamino)butanoyl]amino]ethoxy]ethoxy]acetyl]amino]-ethoxy]ethoxy]acetyl]-[GluA14, HisB25],des-ThrB30-Insulin (human)

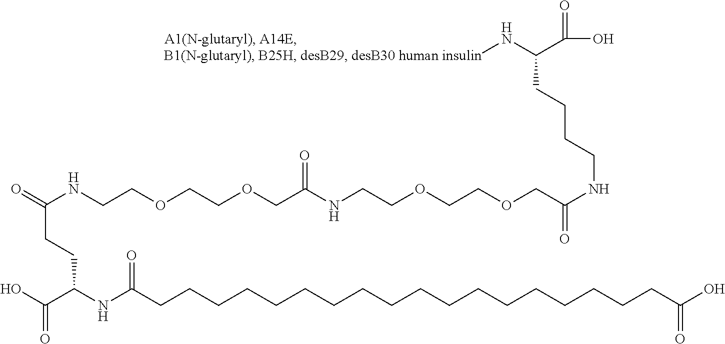

### Example 72

### Insulin Receptor Affinity of Selected Insulin Derivatives of the Invention

The affinity of the acylated insulin analogues of this invention for the human insulin receptor is determined by a SPA assay (Scintillation Proximity Assay) microtiterplate antibody capture assay. SPA-PVT antibody-binding beads, anti-mouse reagent (Amersham Biosciences, Cat No. PRNQ0017) are mixed with 25 ml of binding buffer (100 mM HEPES pH 7.8; 100 mM sodium chloride, 10 mM MgSO₄, 0.025% Tween-20). Reagent mix for a single Packard Optiplate (Packard No. 6005190) is composed of 2.4 μl of a 1:5000 diluted purified recombinant human insulin receptor (either with or without exon 11), an amount of a stock solution of A14Tyr[¹²⁵I]-human insulin corresponding to 5000 cpm per 100 μl of reagent mix, 12 μl of a 1:1000 dilution of F12 antibody, 3 ml of SPA-beads and binding buffer to a total of 12 ml. A total of 100 μl reagent mix is then added to each well in the Packard Optiplate and a dilution series of the insulin derivative is made in the Optiplate from appropriate samples. The samples are then incubated for 16 hours while gently shaken. The phases are the then separated by centrifugation for 1 min and the plates counted in a Topcounter. The binding data were fitted using the nonlinear regression algorithm in the GraphPad Prism 2.01 (GraphPad Software, San Diego, Calif.) and affinities are expressed relative (in percentage (%)) to the affinity of human insulin.

A related assay is also used wherein the binding buffer also contains 1.5% HSA in order to mimic physiological conditions

Insulin receptor affinities and other in vitro data of selected insulins of the invention:

| | | | Lipogenesis in | |
|---|---|---|---|---|
| | Relative IR-A | Relative IR-A | rat adipocytres | Hydropho- |
| | affinity | affinity | (@ 0.1% HSA) | bicity rel. |
| Example | (@ 0% HSA) | (@ 1.5% HSA) | rel. to human | to human |
| No. | (%) | (%)e | insulin | insulin |
| Prior art* | 2.3 | 0.11 | 0.31 | 0.31 |
| 12 | 0.3 | 0.06 | 0.02 | 0.15 |
| 1 | 1.5 | 0.09 | 0.10 | 0.38 |
| 2 | 1.7 | 0.05 | | |
| 13 | 0.3 | 0.02 | | 0.04 |
| 32 | 0.4 | 0.03 | | 0.05 |
| 3 | 0.6 | 0.06 | | 0.08 |
| 35 | 0.8 | 0.16 | | 0.34 |
| 37 | 0.8 | 0.05 | | 0.50 |
| 38 | 0.9 | | | |
| 36 | 0.7 | 0.07 | | |
| 34 | 0.3 | 0.04 | 0.04 | 0.17 |
| 17 | 0.2 | 0.00 | 0.02 | |
| 33 | 0.4 | 0.01 | 0.03 | |
| 4 | 1.7 | 0.03 | 0.12 | 0.27 |
| 5 | 1.7 | 0.17 | 0.13 | 0.30 |
| 39 | 0.6 | 0.01 | | |
| 18 | 0.4 | 0.04 | 0.04 | 0.12 |
| 55 | 0.1 | 0.01 | | |
| 6 | 17.0 | 0.98 | 0.60 | |
| 35 | 2.6 | 0.19 | 0.12 | 0.22 |
| 49 | 3.0 | 0.25 | 0.14 | |
| 40 | 0.5 | 0.05 | 0.05 | |
| 54 | 0.2 | 0.02 | | |
| 10 | | | | |
| 11 | | | | |
| 24 | | | | |
| 23 | 2.7 | 0.04 | | 0.19 |
| 22 | 0.09 | 0.02 | | 0.32 |
| 26 | | | | |
| 46 | | | | |
| 47 | | | | |
| 48 | | | | |
| 50 | | | | |
| 57 | 0.2 | 0.01 | | |
| 19 | 5.0 | 0.14 | | 0.08 |
| 20 | 4.7 | 0.48 | | 0.08 |
| 8 | 2.7 | 0.18 | 1.23 | 0.09 |
| 56 | 1.3 | 0.16 | | 0.10 |
| 9 | 2.5 | 0.33 | 1.34 | 0.10 |
| 61 | | | | |
| 58 | 1.0 | 0.15 | | 0.09 |
| 60 | | | | |
| 59 | | | | |
| 41 | | | | |
| 14 | 0.2 | 0.00 | | |
| 16 | 0.1 | 0.01 | | |
| 15 | 0.2 | 0.02 | | |
| 43 | | | | |
| 7 | 0.5 | 0.03 | | |
| 27 | | | | |
| 42 | | | | |
| 45 | | | | |
| 44 | | | | |
| 28 | 0.7 | 0.25 | | 0.41 |
| 62 | | | | |
| 29 | 2.6 | 0.20 | | 0.71 |
| 63 | | | | |
| 64 | | | | |
| 30 | 0.7 | 0.18 | | 0.64 |
| 65 | | | | |
| 21 | 1.3 | 0.07 | | 0.65 |
| 31 | | | | |
| 51 | | | | |
| 66 | | | | |
| 67 | | | | |
| 52 | | | | |
| 68 | 0.9 | 0.15 | | 0.28 |
| 53 | | | | |
| 69 | 0.1 | 0.02 | | 0.14 |
| 70 | | | | |
| 71 | | | | |

*Prior art = insulin of example 1 without N-terminal modification

### Example 73

### Hydrophobicity of the Insulin Derivatives of the Invention

The hydrophobicity of an insulin derivative is found by reverse phase HPLC run under isocratic conditions. The elution time of the insulin derivative is compared to that of human insulin (herein designated HI) or another derivative with a known hydrophibicity under the same conditions. The hydrophobicity, k′rel, is calculated as: k′rel_{deriv}=((t_{deriv}−t₀)/(t_{ref}−t₀))*k′rel_{ref}. Using HI as reference: k′rel_{ref}=k′rel_{HI}=1. The void time of the HPLC system, t₀, is determined by injecting 5 μl of 0.1 mM NaNO₃. Running conditions:

Column: Lichrosorb RP-C18, 5 μm, 4×250 mm

Buffer A: 0.1 M natrium phosphate pH 7.3, 10 vol % CH₃CN

Buffer B: 50 vol % CH₃CN

Injection volume: 5 μl

Run time: max 60 minutes

After running an initial gradient, the isocratic level for running the derivative and reference (for example HI) is chosen, and the elution times of the derivative and reference under isocratic conditions are used in the above equation to calculate k′rel_{deriv}.

Data are given in the table above.

### Example 74

### Degradation of Insulin Analogs Using Duodenum Lumen Enzymes

Degradation of insulin analogs using duodenum lumen enzymes (prepared by filtration of duodenum lumen content) from SPD rats. The assay is performed by a robot in a 96 well plate (2 ml) with 16 wells available for insulin analogs and standards. Insulin analogs ˜15 μM are incubated with duodenum enzymes in 100 mM Hepes, pH=7.4 at 37° C., samples are taken after 1, 15, 30, 60, 120 and 240 min and reaction quenched by addition of TFA. Intact insulin analogs at each point are determined by RP-HPLC. Degradation half time is determined by exponential fitting of the data and normalized to half time determined for the reference insulins, A14E, B25H, desB30 human insulin or human insulin in each assay. The amount of enzymes added for the degradation is such that the half time for degradation of the reference insulin is between 60 min and 180 min. The result is given as the degradation half time for the insulin analog in rat duodenum divided by the degradation half time of the reference insulin from the same experiment (relative degradation rate). The relative stability of insulins of the invention vs. human insulin is generally 12 fold higher than vs. A14E, B25H, desB30 human insulin.

Data are given in the table below.

| | Duodenum degradation. |
|---|---|
| | Relative stability vs. A14E, |
| Example No. | B25H, desB30 human insulin |
| Prior art* | 0.9 |
| 12 | 1.5 |
| 1 | 1.2 |
| 2 | 0.8 |
| 13 | 1.5 |
| 32 | 1.8 |
| 3 | 3.1 |
| 35 | 0.9 |
| 37 | |
| 38 | |
| 36 | 0.8 |
| 34 | 1.3 |
| 17 | 5.6 |
| 33 | |
| 4 | 8.6 |
| 5 | 6.8 |
| 39 | 4.7 |
| 18 | 5.6 |
| 55 | 1.7 |
| 6 | 3.8 |
| 35 | 1.6 |
| 49 | 3.3 |
| 40 | 4.3 |
| 54 | 6.9 |
| 10 | |
| 11 | |
| 24 | |
| 23 | 6.6 |
| 22 | 7.7 |
| 26 | |
| 46 | |
| 47 | |
| 48 | |
| 50 | |
| 57 | 2.2 |
| 19 | 3.0 |
| 20 | 1.8 |
| 8 | 6.8 |
| 56 | 1.7 |
| 9 | 9.2 |
| 61 | |
| 58 | 1.2 |
| 60 | |
| 59 | |
| 41 | |
| 14 | 0.9 |
| 16 | 0.4 |
| 15 | 0.6 |
| 43 | |
| 7 | 0.4 |
| 27 | |
| 42 | |
| 45 | |
| 44 | |
| 28 | 2.1 |
| 62 | |
| 29 | 3.0 |
| 63 | |
| 64 | |
| 30 | 4.9 |
| 65 | |
| 21 | 6.2 |
| 31 | |
| 51 | |
| 66 | |
| 67 | |
| 52 | |
| 68 | 2.8 |
| 53 | |
| 69 | 1.2 |
| 70 | |
| 71 | |

*Prior art = insulin of example 1 without N-terminal modification

### Example 75

### Lipogenesis in Rat Adipocytes

As a measure of in vitro potency of the insulins of the invention, lipogenesis can be used.

Primary rat adipocytes are isolated from the epididymale fat pads and incubated with 3H-glucose in buffer containing e.g. 0.1% fat free HSA and either standard (human insulin, HI) or insulin of the invention. The labelled glucose is converted into extractable lipids in a dose dependent way, resulting in full dose response curves. The result is expressed as relative potency (%) with 95% confidence limits of insulin of the invention compared to standard (HI).

Data are given in the table above.

### Example 76

### Chemical Stability of Insulin Analogues Formulated in Lipid Formulations

Chemical stability of insulin analogues formulated in lipid formulations was assessed according to the protocol described here. As a comparator the analogue of example 1 without the N-terminal protecting groups was used, denoted “Prior Art” herein.

Composition of the formulation:

Insulin to be tested (75 μM)

15% Propylenglycol

30% Tween 20, Polysorbat 20

55% Diglycerol caprylate

The insulin to be tested (lyophilised from pH 7.5) is dissolved in propylenglycol in the dark for 16 hours. Diglycerol caprylate is added ad the mixture is stirred. Tween 20 is added and the mixture is stirred for 5 minutes. The mixture is gently agitated until it is homogeneous.

Assays:

Extraction:

Extraction-mix: 1-butanol+0.1% (w/w) Tween80, 0.1M Na₂HPO₄/NaH₂PO₄ pH 7.0
1. The formulations are allowed to reach room temperature.
2. To each Eppendorf tube 20 μl of the formulations are added.
3. Add 490 μl 1-butanol followed by addition of 990 μl of the phosphate buffer. Vortex and incubate at RT for 30 min.
4. Vortex again and centrifuge at RT at 14000 rpm for 20 min. Analyse the bottom aqueous phase for purity and HMWP formation.

Alternatively another extraction method can be used:
1. The formulations are allowed to reach room temperature.
2. To each Eppendorf tube 50 μl of the formulations are added.
3. Add 950 μl of extraction buffer. Vortex well. Immediately after, load 800 μl (2×400 μl) for purification on the spin column. See spin protocol below.
   Ion Exchange on Q Spin Columns from Sartorius

Buffers:

Equilibration buffer: 25 mM Na₂HPO₄NaH₂PO₄ pH 7.0

Washing buffer: 100 mM NaCl, 25 mM Na₂HPO₄/NaH₂PO₄ pH 7.0

Elution buffer: 500 mM NaCl, 25 mM Na₂HPO₄/NaH₂PO₄ pH 7.0

Spin Columns:

Vivapure IEX Q spin columns

Spin Protocol:

(In the following all spin steps are for 5 min at 2000×g.)
1. Apply 400 μl equilibration buffer to each spin column, and spin. Discard the flow-through.
2. Apply 2×400 μl of each extracted sample. Spin the column between each application. Discard the flow-through.
3. Apply 400 μl washing buffer to wash each spin column, and spin. Discard the wash.
4. Apply 400 μl elution buffer to each spin column, and spin. Analyse the elution for purity and HMWP formation

### Purity Method:

Parameters:

Column: Waters BEH Shield RP18 UPLC column (2.1×100 mm, 1.7 μm)

Wavelength: 215 nm

Column temperature: 50° C.

Flow: 0.4 ml/min

Run time: 18.5 min

Load: 7.5 μl

Buffer A: 0.09M di-ammonium hydrogen phosphate pH 3.0, 10% (v/v) acetonitrile

Buffer B: 90% acetonitrile.

| Time (min) | Flow (ml/min) | % A | % B |
|---|---|---|---|
| Initial | 0.400 | 73.0 | 27.0 |
| 1.00 | 0.400 | 73.0 | 27.0 |
| 2.50 | 0.400 | 68.0 | 32.0 |
| 12.00 | 0.400 | 50.0 | 50.0 |
| 13.50 | 0.400 | 20.0 | 80.0 |
| 15.00 | 0.400 | 20.0 | 80.0 |
| 17.00 | 0.400 | 73.0 | 27.0 |
| 19.00 | End | End | End |

### HMWP Method:

Parameters:

Column: Waters Insulin HMWP SEC column

Wavelength: 215 nm

Column temperature: 50° C.

Flow: 0.5 ml/min

Run-time: 30 min

Load: 40 μl

Buffer: 500 mM NaCl, 10 mM NaH₂PO₄, 5 mM H₃PO₄, 50% (v/v) 2-propanol

Overview Over Impurities and HMWP Formed after 2 and 4 Weeks at 25/30° C.:

| | Impurities formed (%) | | HMWP formed (%) | | |
|---|---|---|---|---|---|
| Example | 2 weeks | 4 weeks | 2 weeks | 4 weeks | Note |
| Prior art | 24.0 | 33.8 | 4.4 | 7.2 | 25° C. |
| 1 | — | 9.8 | — | 0.4 | 30° C. |
| 2 | 1.7 | 3.2 | 0.0 | 0.1 | 25° C. |
| 12 | — | 4.4 | — | 0.2 | 30° C. |
| 33 | — | 0.0 | — | 0.8 | 30° C. |
| 38 | — | 7.2 | — | 0.5 | 30° C. |
| 39 | — | 9.6 | — | 0.3 | 30° C. |
| 40 | 2.1 | — | 0.2 | — | 25° C. |
| 41 | 3.8 | — | 0.1 | — | 25° C. |
| 59 | 2.4 | — | 0.1 | — | 25° C. |
| 60 | 3.3 | — | 0.1 | — | 25° C. |

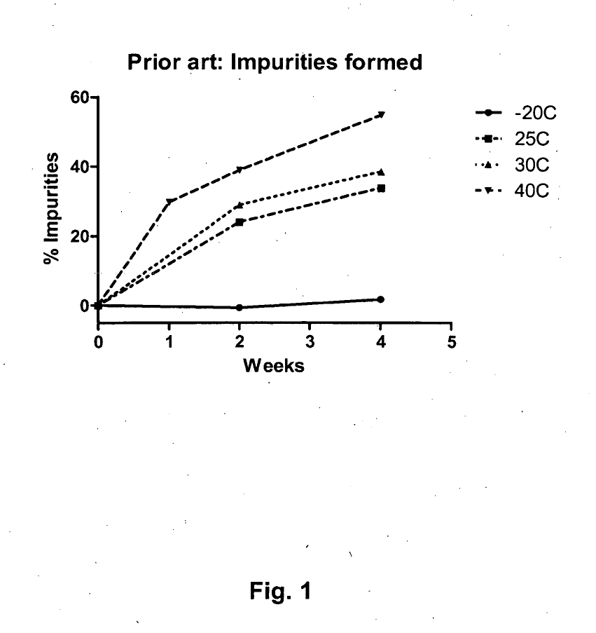

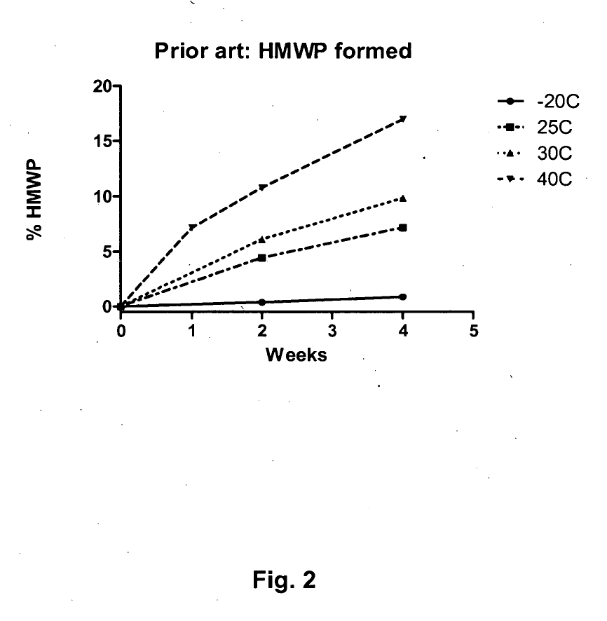

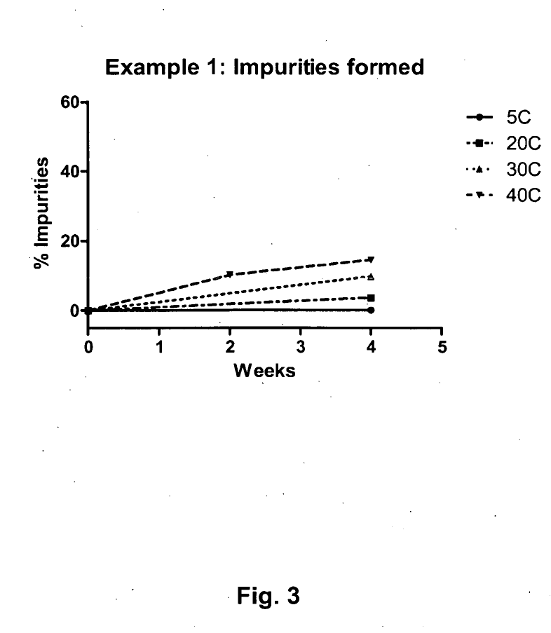

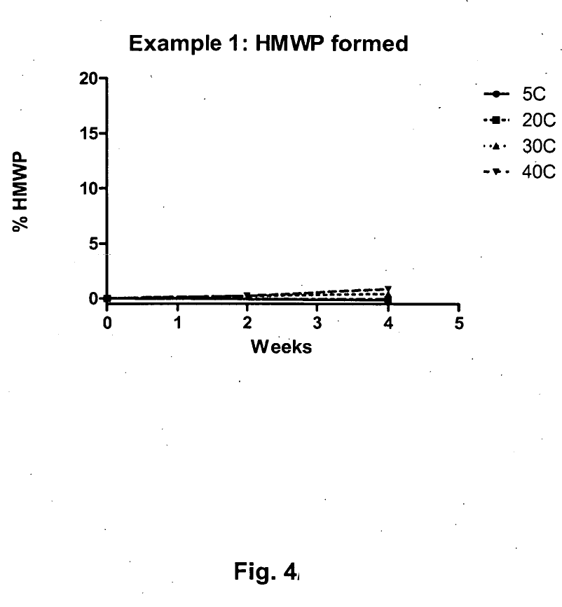

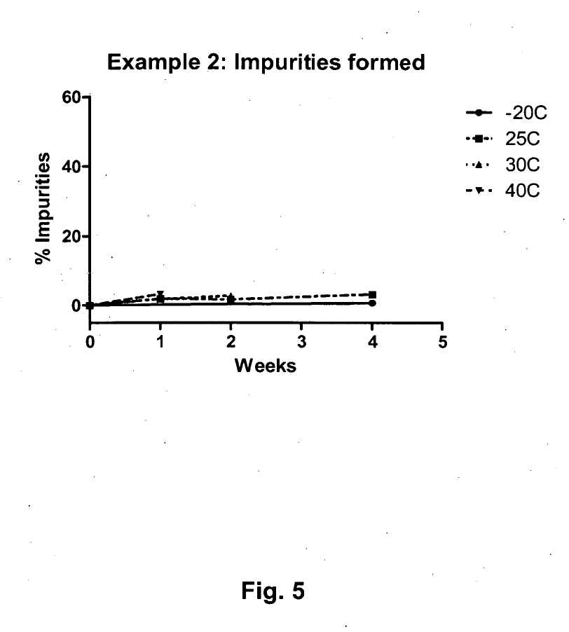

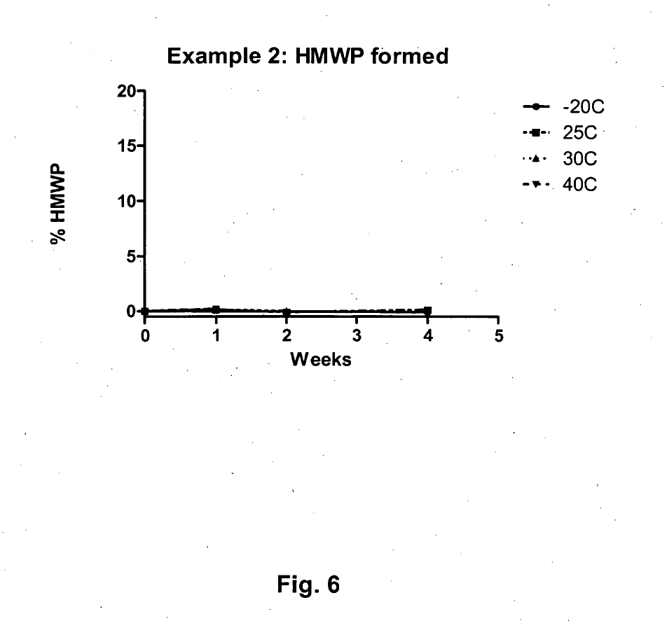

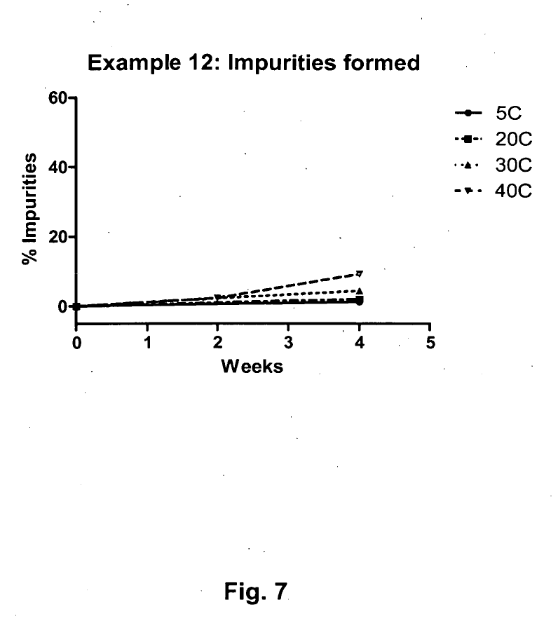

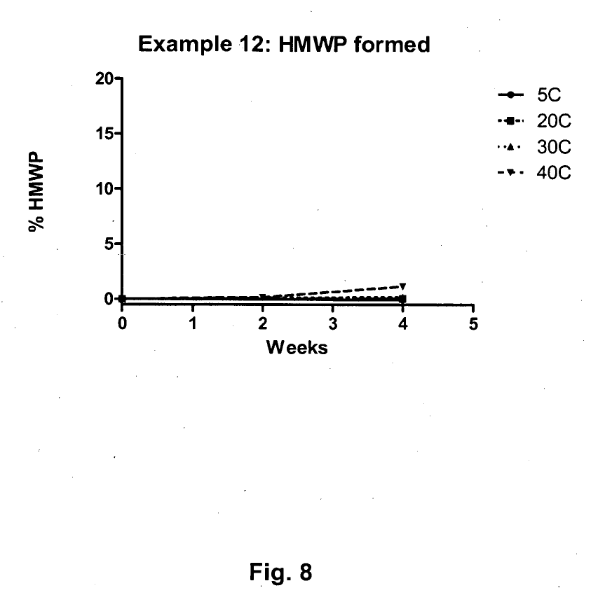

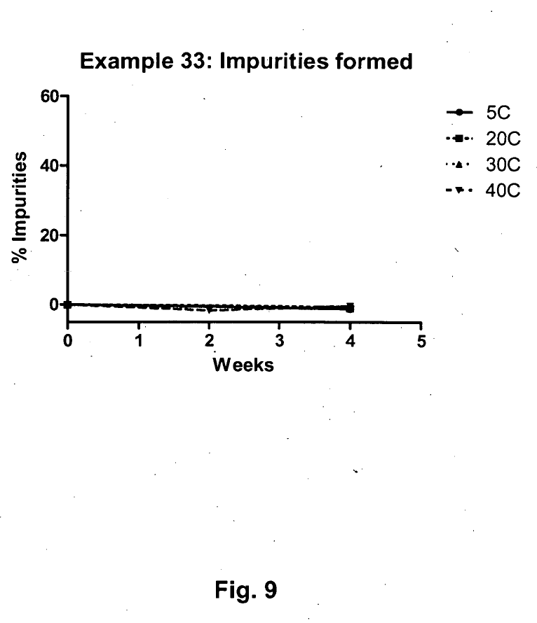

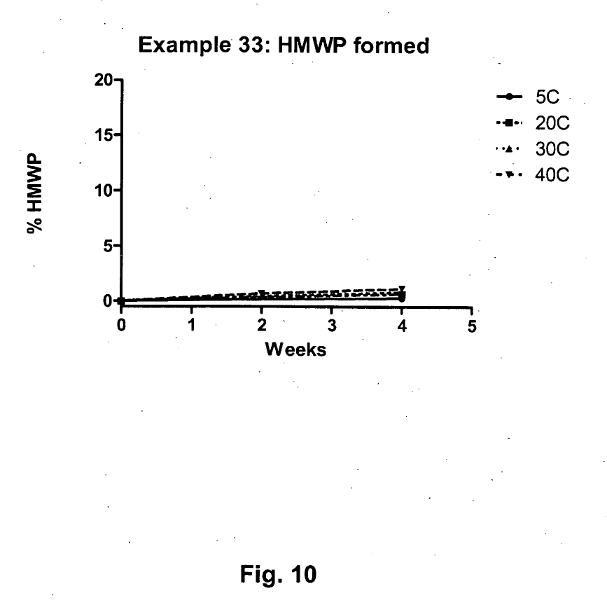

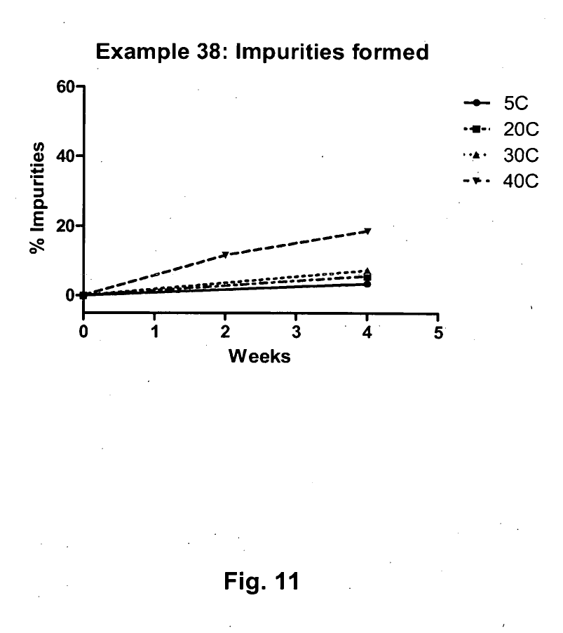

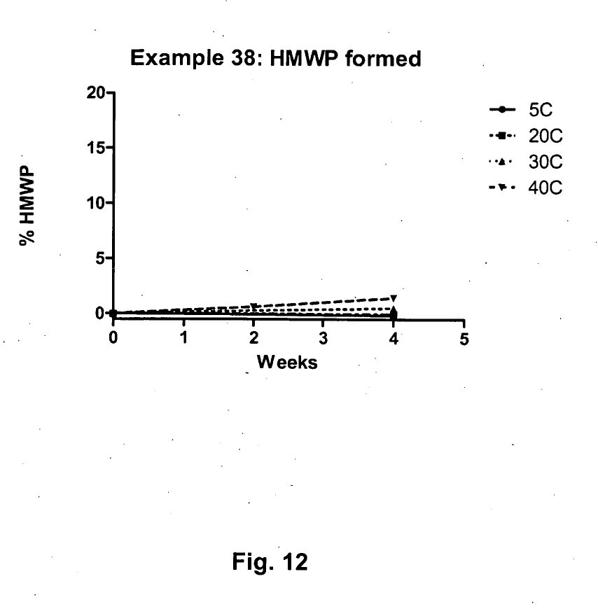

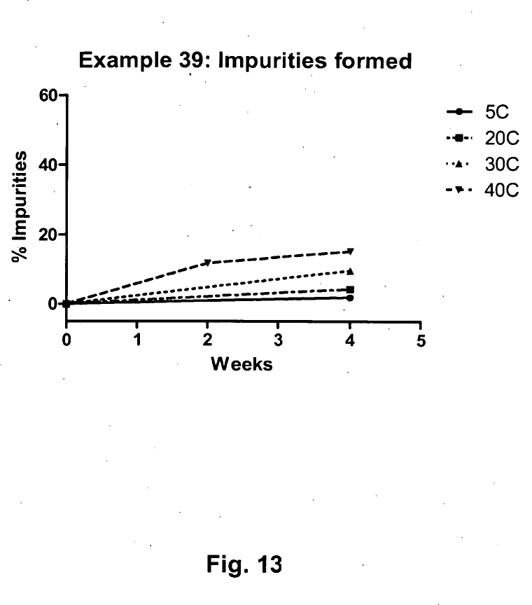

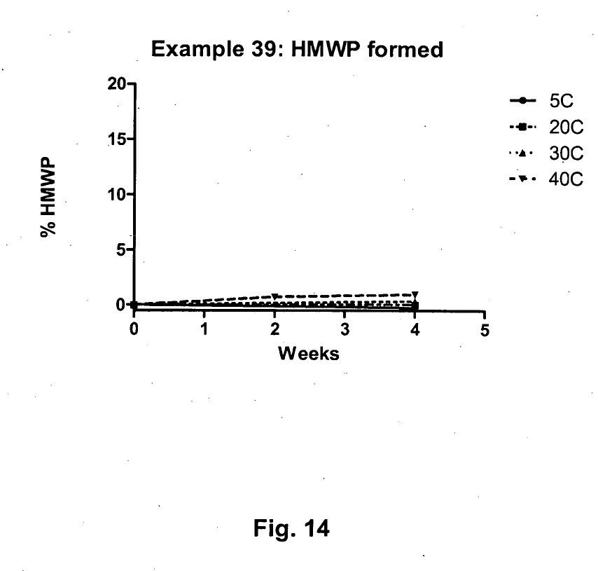

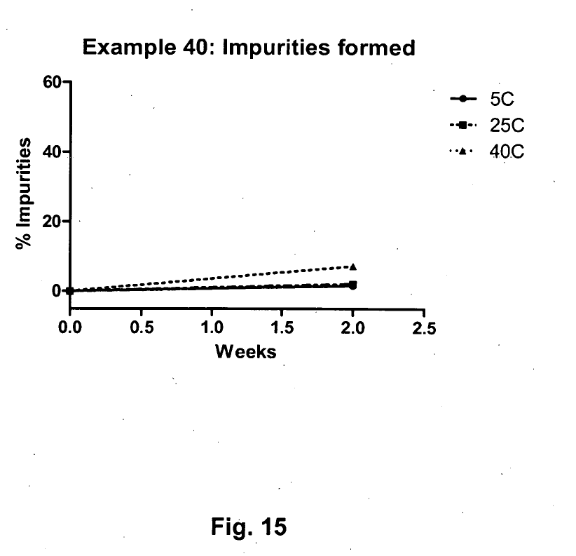

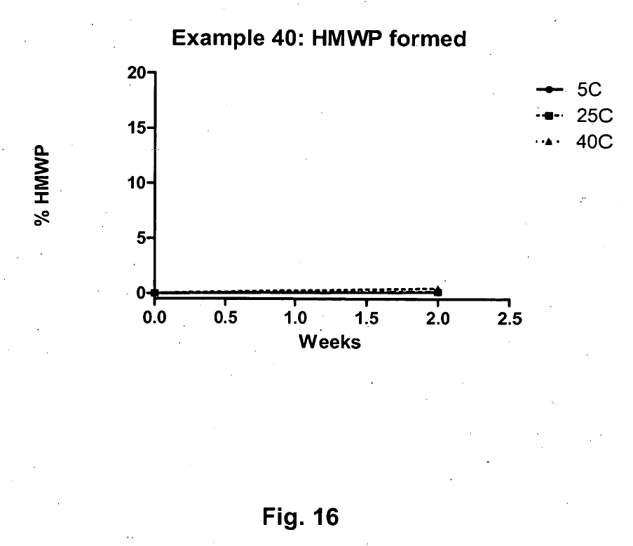

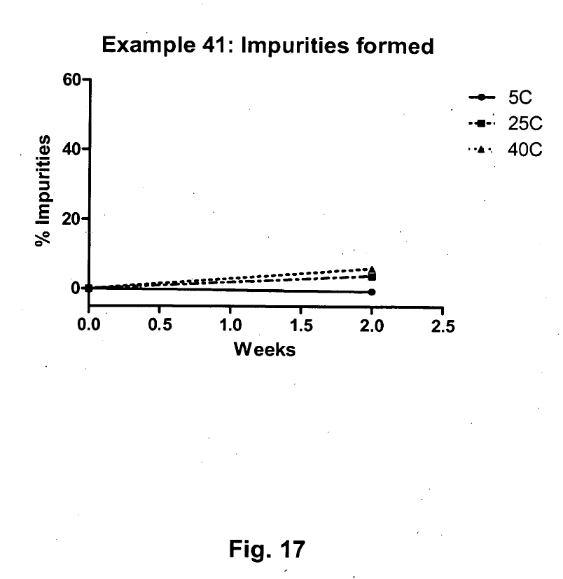

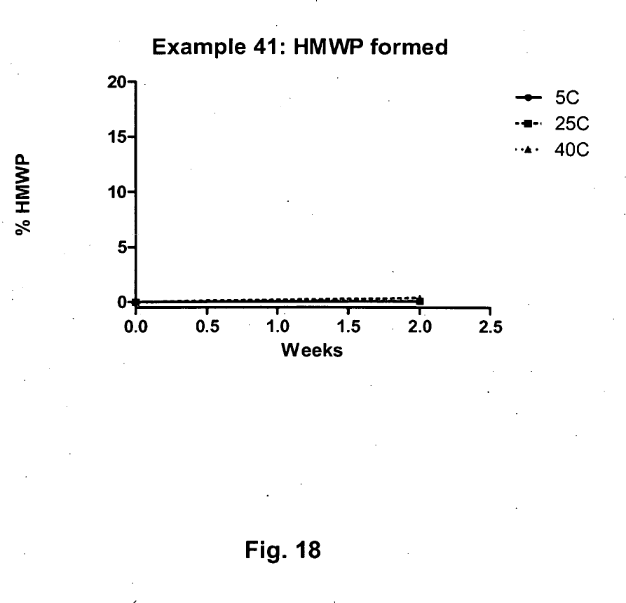

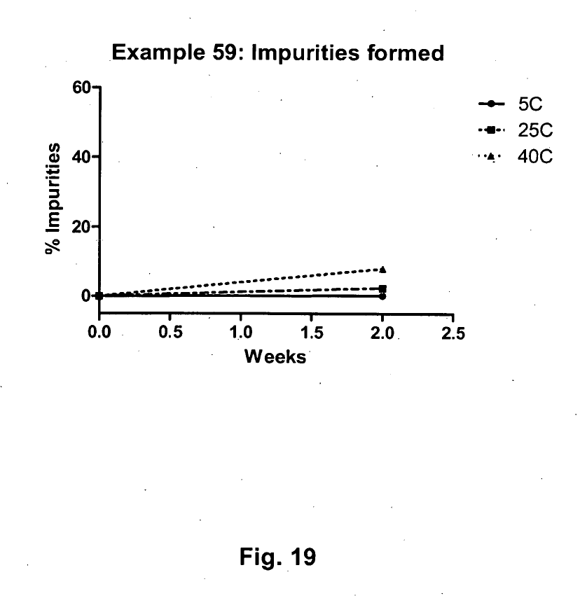

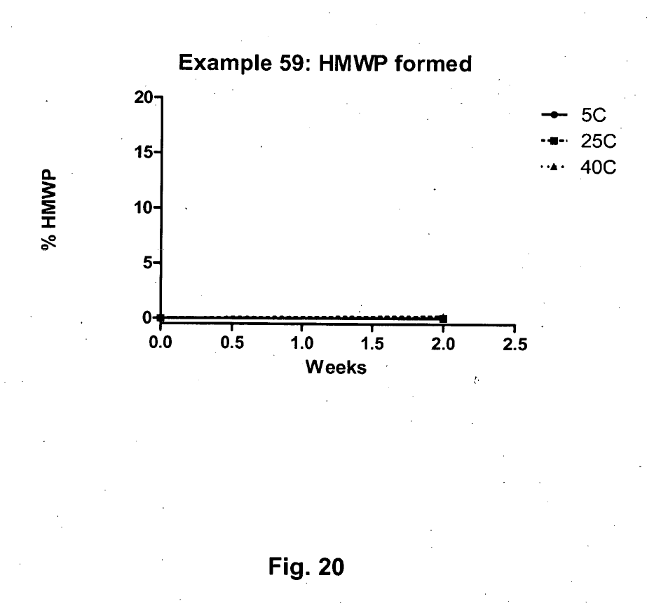

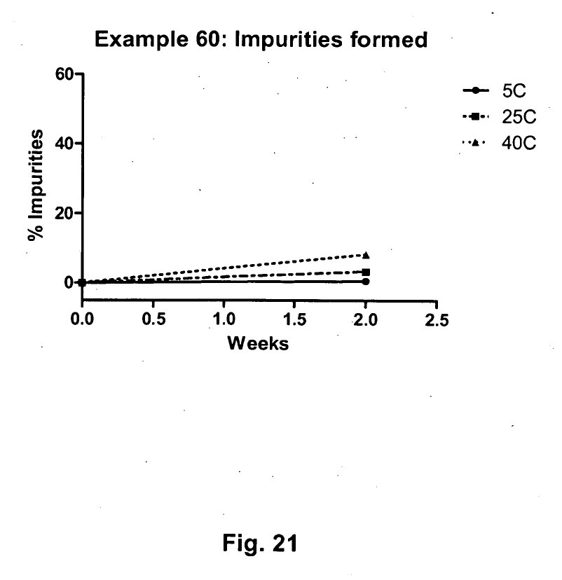

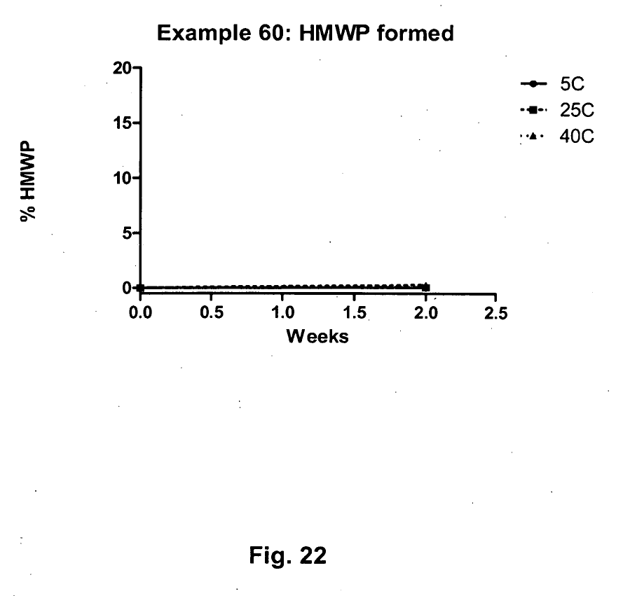

Results of the chemical stability studies are furthermore shown in FIGS. 1-22.

### Example 77

### Rat pharmacokinetics, Intravenous Rat PK

Anaesthetized rats are dosed intravenously (i.v.) with insulin analogs at various doses and plasma concentrations of the employed compounds are measured using immunoassays or mass spectrometry at specified intervals for 4-6 or up to 48 hours or more post-dose. Pharmacokinetic parameters are subsequently calculated using WinNonLin Professional (Pharsight Inc., Mountain View, Calif., USA).

Non-fasted male Wistar rats (Taconic) weighing approximately 200 gram are used.

Body weight is measured and rats are subsequently anaesthetized with Hypnorm/Dormicum (each compound is separately diluted 1:1 in sterile water and then mixed; prepared freshly on the experimental day). Anesthesia is initiated by 2 ml/kg Hypnorm/Doricum mixture sc followed by two maintenance doses of 1 ml/kg sc at 30 min intervals and two maintenance doses of 1 ml/kg sc with 45 min intervals. If required in order to keep the rats lightly anaesthetised throughout a further dose(s) 1-2 ml/kg sc is supplied. Weighing and initial anaesthesia is performed in the rat holding room in order to avoid stressing the animals by moving them from one room to another.

### Example 78

### Rat pharmacokinetics, Rat PK Following Intraintestinal Injection

Anaesthetized rats are dosed intraintestinally (into jejunum) with insulin analogs. Plasma concentrations of the employed compounds as well as changes in blood glucose are measured at specified intervals for 4 hours or more post-dosing. Pharmacokinetic parameters are subsequently calculated using WinNonLin Professional (Pharsight Inc., Mountain View, Calif., USA).

Male Sprague-Dawley rats (Taconic), weighing 250-300 g, fasted for ˜18 h are anesthetized using Hypnorm-Dormicum s.c. (0.079 mg/ml fentanyl citrate, 2.5 mg/ml fluanisone and 1.25 mg/ml midazolam) 2 ml/kg as a priming dose (to timepoint −60 min prior to test substance dosing), 1 ml/kg after 20 min followed by 1 ml/kg every 40 min.

The insulins to be tested in the intraintestinal injection model are formulated as formulated for the gavage model above.

The anesthetized rat is placed on a homeothermic blanket stabilized at 37° C. A 20 cm polyethylene catheter mounted a 1-ml syringe is filled with insulin formulation or vehicle. A 4-5 cm midline incision is made in the abdominal wall. The catheter is gently inserted into mid-jejunum ˜50 cm from the caecum by penetration of the intestinal wall. If intestinal content is present, the application site is moved ±10 cm. The catheter tip is placed approx. 2 cm inside the lumen of the intestinal segment and fixed without the use of ligatures. The intestines are carefully replaced in the abdominal cavity and the abdominal wall and skin are closed with autoclips in each layer. At time 0, the rats are dosed via the catheter, 0.4 ml/kg of test compound or vehicle.

Blood samples for the determination of whole blood glucose concentrations are collected in heparinised 10 μl capillary tubes by puncture of the capillary vessels in the tail tip. Blood glucose concentrations are measured after dilution in 500 μl analysis buffer by the glucose oxidase method using a Biosen autoanalyzer (EKF Diagnostic Gmbh, Germany). Mean blood glucose concentration courses (mean±SEM) are made for each compound.

Samples are collected for determination of the plasma insulin concentration. 100 μl blood samples are drawn into chilled tubes containing EDTA. The samples are kept on ice until centrifuged (7000 rpm, 4° C., 5 min), plasma is pipetted into Micronic tubes and then frozen at 20° C. until assay. Plasma concentrations of the insulin analogs are measured in a immunoassay which is considered appropriate or validated for the individual analog.

Blood samples are drawn at t=−10 (for blood glucose only), at t=−1 (just before dosing) and at specified intervals for 4 hours or more post-dosing.

### Example 79

### Potency of the Acylated Insulin Analogues of this Invention Relative to Human insulin

Sprague Dawley male rats weighing 238-383 g on the experimental day are used for the clamp experiment. The rats have free access to feed under controlled ambient conditions and are fasted overnight (from 3 pm) prior to the clamp experiment.

Experimental Protocol:

The rats are acclimatized in the animal facilities for at least 1 week prior to the surgical procedure. Approximately 1 week prior to the clamp experiment, Tygon catheters are inserted under halothane anaesthesia into the jugular vein (for infusion) and the carotid artery (for blood sampling) and exteriorised and fixed on the back of the neck. The rats are given Streptocilin vet. (Boehringer Ingelheim; 0.15 ml/rat, i.m.) post-surgically and placed in an animal care unit (25° C.) during the recovery period. In order to obtain analgesia, Anorphin (0.06 mg/rat, s.c.) is administered during anaesthesia and Rimadyl (1.5 mg/kg, s.c.) is administered after full recovery from the anaesthesia (2-3 h) and again once daily for 2 days.

At 7 am on the experimental day overnight fasted (from 3 pm the previous day) rats are weighed and connected to the sampling syringes and infusion system (Harvard 22 Basic pumps, Harvard, and Perfectum Hypodermic glass syringe, Aldrich) and then placed into individual clamp cages where they rest for ca. 45 min before start of experiment. The rats are able to move freely on their usual bedding during the entire experiment and have free access to drinking water. After a 30 min basal period during which plasma glucose levels were measured at 10 min intervals, the insulin derivative to be tested and human insulin (one dose level per rat, n=6-7 per dose level) are infused (i.v.) at a constant rate for 300 min. Optionally a priming bolus infusion of the insulin derivative to be tested is administered in order to reach immediate steady state levels in plasma. The dose of the priming bolus infusion can be calculated based on clearance data obtained from i.v. bolus pharmacokinetics by a pharmacokinetician skilled in the art. Plasma glucose levels are measured at 10 min intervals throughout and infusion of 20% aqueous glucose is adjusted accordingly in order to maintain euglyceamia. Samples of re-suspended erythrocytes are pooled from each rat and returned in about ½ ml volumes via the carotid catheter.

On each experimental day, samples of the solutions of the individual insulin derivatives to be tested and the human insulin solution are taken before and at the end of the clamp experiments and the concentrations of the peptides are confirmed by HPLC. Plasma concentrations of rat insulin and C-peptide as well as of the insulin derivative to be tested and human insulin are measured at relevant time points before and at the end of the studies. Rats are killed at the end of experiment using a pentobarbital overdose.

### Example 80

### Potency of the Acylated Insulin Derivatives of this Invention Relative to a Control Insulin Derivative, Subcutaneous Administration to Rats

Male Sprague-Dawley rats (n=6 per group) receives a single dose subcutaneously of vehicle or insulin insulin analogue (50 or 200 nmol/animal for analogues with a medium duration of action or long duration of action, respectively). Blood (sublingual) is drawn and plasma collected at time points 0, 1, 2, 4, 8, 24 and 48 or 0, 2, 4, 8, 24, 48, 72, 96 hours after dosing, for analogues with a medium duration of action or long duration of action, respectively). Plasma is assayed for glucose. The glucose lowering effect is calculated as the area under the curve of −delta plasma glucose as a function of time and compared to a control insulin derivative.

### Example 81

### Dog Pharmacokinecics, Intravenous Dog PK

Male Beagle dogs (approximately 12 kg) receives a single dose intravenously of insulin insulin analogue (2 nmol/kg). Blood is drawn and plasma collected at time points—0.17, 0, 0.083, 0.25, 0.5, 0.75, 1, 1.25, 1.5, 2, 2.5, 3, 3.5, 4, 5, 8, 10, 12, 16, 24, 32, 48, 72, 96, 120, 144 and 168 hours after dosing. Plasma samples are analyzed by either sandwich immunoassay or LCMS. Plasma concentration-time profiles are analysed by non-compartmental pharmacokinetics analysis using WinNonlin Professional 5.2 (Phar-sight Inc., Mountain View, Calif., USA). Simultaneous measurements of blood or plasma glucose may also be performed.

### Example 82

### Dog Pharmacokinecics, Oral Dosing

Male Beagle dogs (approximately 12 kg) receives a single dose orally of insulin analogue (120 nmol/kg) formulated in an enteric coated capsule, size 00. Blood is drawn and plasma collected at time points 0, 15, 30, 45, 60, 75, 90, 105, 120, 135, 150, 165, 180, 210, 240, 270, 300, 360, 480, 600, 720, 1440 minutes (24 h), 30 h, 48 h and 72 h after dosing. Plasma samples are analyzed by either sandwich immunoassay or LCMS. Plasma concentration-time profiles are analysed by non-compartmental pharmacokinetics analysis using WinNonlin Professional 5.2 (Phar-sight Inc., Mountain View, Calif., USA). Simultaneous measurements of blood or plasma glucose may also be performed.

1. An N-terminally modified insulin, wherein the insulin is an acylated, protease stabilised insulin and the N-terminal modification is with one or more N-terminal modification groups that are positively charged at physiological pH.

2. An N-terminally modified insulin according to claim 1, wherein the N-terminally modified insulin consists of a peptide part, a lipophilic substituent and an N-terminal modification group.

3. An N-terminally modified insulin according to claim 1, wherein the positively charged modification groups at physiological pH are one or two organic substituents which are positively charged at physiological pH and are having a MW below 200 g per mol conjugated to the N-terminals of the parent insulin.

4. An N-terminally modified insulin according to claim 1, wherein the positively charged modification groups at physiological pH are designated Y and Z in 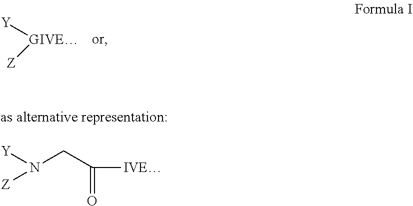
wherein Y and Z are attached to at the N-terminal amino acids of the insulin peptide.

5. An N-terminally modified insulin according to claim 1, wherein the acylated, protease stabilised insulin consists of a protease stabilised insulin as peptide part and a lipophilic substituent attached to the peptide part, wherein the peptide part is human insulin substituted such that at least one hydrophobic amino acid has been substituted with hydrophilic amino acids, and wherein said substitution is within or in close proximity to one or more protease cleavage sites of the insulin.

6. An N-terminally modified insulin, wherein the insulin is an acylated insulin and the N-terminal modification is with one or more N-terminal modification groups that are neutral or negatively charged at physiological pH.

7. An N-terminally modified insulin according to claim 6, wherein the N-terminally modified insulin consists of a peptide part, a lipophilic substituent and an N-terminal modification group.

8. An N-terminally modified insulin according to claim 6, wherein the neutral or negatively charged modification groups at physiological pH are one or two organic substituents which are neutral or negatively charged at physiological pH and are having a MW below 200 g per mol conjugated to the N-terminal of the parent insulin.

9. An N-terminally modified insulin according to claim 6, wherein the N-terminal modification is selected from the group consisting of: Carbamoyl, thiocarbamoyl, C1-C4 chain acyl groups, oxalyl, glutaryl and diglycolyl.

10. An N-terminally modified insulin according to claim 6, wherein the acylated insulin consists of a peptide part and a lipophilic substituent attached to the peptide part, wherein the peptide part is human insulin, desB30 human insulin, human insulin with less than 8 modifications or desB30 human insulin with less than 8 modifications.

11. An oral pharmaceutical composition comprising one or more lipids and an N-terminally modified insulin.

12. An oral pharmaceutical composition according to claim 11, wherein the N-terminally modified insulin consists of a peptide part, an N-terminal modification group and optionally a lipophilic substituent.

13. An oral pharmaceutical composition according to claim 11, which is a solid or semi-solid pharmaceutical composition comprising an N-terminally modified insulin (a), at least one polar organic solvent (b) for the N-terminally modified insulin, at least one surfactant (c), at least one lipophilic component (d), and optionally at least one solid hydrophilic component (e), wherein said pharmaceutical composition is spontaneously dispersible.

14. An oral pharmaceutical composition according to claim 11, which is a water-free liquid pharmaceutical composition comprising an N-terminally modified insulin (a), at least one polar organic solvent (b) for the N-terminally modified insulin, at least one lipophilic component (c), and optionally at least one surfactant (d), wherein the pharmaceutical composition is in the form of a clear solution.

15. An oral pharmaceutical composition according to claim 11, wherein the N-terminally modified insulin has a peptide part which is human insulin, desB30 human insulin, human insulin with less than 8 modifications or desB30 human insulin with less than 8 modifications.
